SEQUENCE LISTING (523 sequences)

SEQ ID NO: 1 - protein, 10 aa, synthetic construct
GFTVSNSVMA

SEQ ID NO: 2 - protein, 16 aa, synthetic construct
IINSIGITNYADSVKG

SEQ ID NO: 3 - protein, 6 aa, synthetic construct
NFDRIY

SEQ ID NO: 4 - protein, 114 aa, synthetic construct
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSS

SEQ ID NO: 5 - protein, 8 aa, synthetic construct
GSIFSINA

SEQ ID NO: 6 - protein, 7 aa, synthetic construct
FTGDTNT

SEQ ID NO: 7 - protein, 14 aa, synthetic construct
AADVQLFSRDYEFY

SEQ ID NO: 8 - protein, 120 aa, synthetic construct
EVQLVESGGGEVQPGGSLRLSCAASGSIFSINAMGWYRQAPGKQRELVAGFTGDTNTIYAESVKGRFTISRDNAKNTVYLQMSSLRAEDTAVYYCAADVQLFSRDYEFYWGQGTLVTVKP

SEQ ID NO: 9-10 - skipped (2 entries)

SEQ ID NO: 11 - protein, 216 aa, synthetic construct
APELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 216 aa, synthetic construct
APELLGGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 15 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHATQKSLSLSPG

SEQ ID NO: 16 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 17 - protein, 216 aa, synthetic construct
APEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 216 aa, synthetic construct
APEAEGAPSVFLFPPKPKDTLMISRTPEVTCVVVDVSHEDPEVKFNWYVDGVEVHNAKTKPREEQYNSTYRVVSVLTVLAQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 216 aa, synthetic construct
APEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 20 - protein, 216 aa, unidentified
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 8 aa, synthetic construct
GYIFNSCG

SEQ ID NO: 22 - protein, 7 aa, synthetic construct
ISGDGDT

SEQ ID NO: 23 - protein, 9 aa, synthetic construct
AVCYNLETY

SEQ ID NO: 24 - protein, 115 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 25-39 - skipped (15 entries)

SEQ ID NO: 40 - protein, 15 aa, unidentified
EPKSCDKTHTCPPCP

SEQ ID NO: 41 - protein, 15 aa, unidentified
EPKSSDKTHTCPPCP

SEQ ID NO: 42 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 231 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 44-100 - skipped (57 entries)

SEQ ID NO: 101 - protein, 114 aa, synthetic construct
QVQYVESGGGLVQPGGSLRLSCAASGFTFSSYRINWYRQPPGKGRELVGSITDTGSTNYADSVKGRFTISRDNAKNTVYLQMSSLKPEDTAVYYCRAPTIAAYWGQGTQVTVSS

SEQ ID NO: 102 - protein, 114 aa, synthetic construct
QVQLVESGGGLVQPGGSLRLSCAASGFTFSSYRINWFRQAPGQGLEAVASITDTGSTNYADSVKGRFTISRDNSKNTLYLQMNSLRAEDTAVYYCAAPTIAAYWGQGTLVTVSS

SEQ ID NO: 103 - protein, 114 aa, synthetic construct
QVQLVESGGGLVQPGGSLRLSCAASGFTFSSYRINWFRQAPGQGLELVASITDTGSTNYADSVKGRFTISRDNSKNTLYLQMNSLRAEDTAVYYCAAPTIAAYWGQGTLVTVSS

SEQ ID NO: 104 - protein, 114 aa, synthetic construct
QVQLVESGGGLVQPGGSLRLSCAASGFTFSSYRINWYRQAPGQGLELVASITDTGSTNYADSVKGRFTISRDNSKNTLYLQMNSLRAEDTAVYYCAAPTIAAYWGQGTLVTVSS

SEQ ID NO: 105 - protein, 114 aa, synthetic construct
QVQLVESGGGLVQPGGSLRLSCAASGFTFSSYRINWYRQAPGQGRELVASITDTGSTNYADSVKGRFTISRDNSKNTLYLQMNSLRAEDTAVYYCAAPTIAAYWGQGTLVTVSS

SEQ ID NO: 106 - protein, 114 aa, synthetic construct
QVQLVESGGGLVQPGGSLRLSCAASGFTFSSYRINWYRQPPGKGRELVGSITDTGSTNYADSVKGRFTISRDNSKNTLYLQMNSLRAEDTAVYYCAAPTIAAYWGQGTLVTVSS

SEQ ID NO: 107 - protein, 8 aa, synthetic construct
GFTFSSYR

SEQ ID NO: 108 - protein, 5 aa, synthetic construct
SYRIN

SEQ ID NO: 109 - protein, 7 aa, synthetic construct
GFTFSSY

SEQ ID NO: 110 - protein, 7 aa, synthetic construct
ITDTGST

SEQ ID NO: 111 - protein, 16 aa, synthetic construct
SITDTGSTNYADSVKG

SEQ ID NO: 112 - protein, 5 aa, synthetic construct
TDTGS

SEQ ID NO: 113 - protein, 8 aa, synthetic construct
AAPTIAAY

SEQ ID NO: 114 - protein, 6 aa, synthetic construct
PTIAAY

SEQ ID NO: 115 - protein, 6 aa, synthetic construct
PTIAAY

SEQ ID NO: 116 - protein, 345 aa, synthetic construct
QVQLVESGGGLVQPGGSLRLSCAASGFTFSSYRINWFRQAPGQGLEAVASITDTGSTNYADSVKGRFTISRDNSKNTLYLQMNSLRAEDTAVYYCAAPTIAAYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 117 - protein, 345 aa, synthetic construct
QVQLVESGGGLVQPGGSLRLSCAASGFTFSSYRINWFRQAPGQGLELVASITDTGSTNYADSVKGRFTISRDNSKNTLYLQMNSLRAEDTAVYYCAAPTIAAYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 118 - protein, 345 aa, synthetic construct
QVQLVESGGGLVQPGGSLRLSCAASGFTFSSYRINWYRQAPGQGLELVASITDTGSTNYADSVKGRFTISRDNSKNTLYLQMNSLRAEDTAVYYCAAPTIAAYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 119 - protein, 345 aa, synthetic construct
QVQLVESGGGLVQPGGSLRLSCAASGFTFSSYRINWYRQAPGQGRELVASITDTGSTNYADSVKGRFTISRDNSKNTLYLQMNSLRAEDTAVYYCAAPTIAAYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 120 - protein, 345 aa, synthetic construct
QVQLVESGGGLVQPGGSLRLSCAASGFTFSSYRINWYRQPPGKGRELVGSITDTGSTNYADSVKGRFTISRDNSKNTLYLQMNSLRAEDTAVYYCAAPTIAAYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 121-200 - skipped (80 entries)

SEQ ID NO: 201 - protein, 117 aa, synthetic construct
QVQLVESGGGSVQPGGSLRLSCAASGIVFNSDVMGWYRQVPGKPRELVATITGGGSTNYATSVKGRFTISRDNAKNTVYLQMNSLRPEDTAVYYCNGRRGDSMLAFWAQGTQVTVSS

SEQ ID NO: 202 - protein, 117 aa, synthetic construct
QVQLVESGGGLVQPGGSLRLSCAASGIVFNSDVMGWFRQAPGQGLEAVATITGGGSTNYATSVKGRFTISRDNSKNTLYLQMNSLRAEDTAVYYCAARRGDSMLAFWGQGTLVTVSS

SEQ ID NO: 203 - protein, 117 aa, synthetic construct
QVQLVESGGGLVQPGGSLRLSCAASGIVFNSDVMGWFRQAPGQGLELVATITGGGSTNYATSVKGRFTISRDNSKNTLYLQMNSLRAEDTAVYYCAARRGDSMLAFWGQGTLVTVSS

SEQ ID NO: 204 - protein, 117 aa, synthetic construct
QVQLVESGGGLVQPGGSLRLSCAASGIVFNSDVMGWYRQAPGQGLELVATITGGGSTNYATSVKGRFTISRDNSKNTLYLQMNSLRAEDTAVYYCAARRGDSMLAFWGQGTLVTVSS

SEQ ID NO: 205 - protein, 117 aa, synthetic construct
QVQLVESGGGLVQPGGSLRLSCAASGIVFNSDVMGWYRQAPGQPRELVATITGGGSTNYATSVKGRFTISRDNSKNTLYLQMNSLRAEDTAVYYCAARRGDSMLAFWGQGTLVTVSS

SEQ ID NO: 206 - protein, 117 aa, synthetic construct
QVQLVESGGGLVQPGGSLRLSCAASGIVFNSDVMGWYRQVPGKPRELVATITGGGSTNYATSVKGRFTISRDNSKNTLYLQMNSLRAEDTAVYYCAARRGDSMLAFWGQGTLVTVSS

SEQ ID NO: 207 - protein, 8 aa, synthetic construct
GIVFNSDV

SEQ ID NO: 208 - protein, 5 aa, synthetic construct
SDVMG

SEQ ID NO: 209 - protein, 7 aa, synthetic construct
GIVFNSD

SEQ ID NO: 210 - protein, 7 aa, synthetic construct
ITGGGST

SEQ ID NO: 211 - protein, 16 aa, synthetic construct
TITGGGSTNYATSVKG

SEQ ID NO: 212 - protein, 5 aa, synthetic construct
TGGGS

SEQ ID NO: 213 - protein, 11 aa, synthetic construct
AARRGDSMLAF

SEQ ID NO: 214 - protein, 9 aa, synthetic construct
RRGDSMLAF

SEQ ID NO: 215 - protein, 9 aa, synthetic construct
RRGDSMLAF

SEQ ID NO: 216 - protein, 348 aa, synthetic construct
QVQLVESGGGLVQPGGSLRLSCAASGIVFNSDVMGWFRQAPGQGLEAVATITGGGSTNYATSVKGRFTISRDNSKNTLYLQMNSLRAEDTAVYYCAARRGDSMLAFWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 217 - protein, 348 aa, synthetic construct
QVQLVESGGGLVQPGGSLRLSCAASGIVFNSDVMGWFRQAPGQGLELVATITGGGSTNYATSVKGRFTISRDNSKNTLYLQMNSLRAEDTAVYYCAARRGDSMLAFWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 218 - protein, 348 aa, synthetic construct
QVQLVESGGGLVQPGGSLRLSCAASGIVFNSDVMGWYRQAPGQGLELVATITGGGSTNYATSVKGRFTISRDNSKNTLYLQMNSLRAEDTAVYYCAARRGDSMLAFWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 219 - protein, 348 aa, synthetic construct
QVQLVESGGGLVQPGGSLRLSCAASGIVFNSDVMGWYRQAPGQPRELVATITGGGSTNYATSVKGRFTISRDNSKNTLYLQMNSLRAEDTAVYYCAARRGDSMLAFWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 220 - protein, 348 aa, synthetic construct
QVQLVESGGGLVQPGGSLRLSCAASGIVFNSDVMGWYRQVPGKPRELVATITGGGSTNYATSVKGRFTISRDNSKNTLYLQMNSLRAEDTAVYYCAARRGDSMLAFWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 221-300 - skipped (80 entries)

SEQ ID NO: 301 - protein, 122 aa, synthetic construct
QVQLVESGGGLVQAGDSLRLSCVASDRTFSSYAVGWFRQAPGKEREFVAAISWNGGRTLYTDSVTGRFTISRDNAKSTVYLQMNGLKPEDTAVYYCAARPAAPTRRLEYDYWGQGTQVTVSS

SEQ ID NO: 302 - protein, 122 aa, synthetic construct
QVQLVESGGGLVQPGGSLRLSCAASDRTFSSYAVGWFRQAPGQGLEAVAAISWNGGRTLYTDSVTGRFTISRDNSKNTLYLQMNSLRAEDTAVYYCAARPAAPTRRLEYDYWGQGTLVTVSS

SEQ ID NO: 303 - protein, 122 aa, synthetic construct
QVQLVESGGGLVQPGGSLRLSCAASDRTFSSYAVGWFRQAPGQGLEFVAAISWNGGRTLYTDSVTGRFTISRDNSKNTLYLQMNSLRAEDTAVYYCAARPAAPTRRLEYDYWGQGTLVTVSS

SEQ ID NO: 304 - protein, 122 aa, synthetic construct
QVQLVESGGGLVQPGGSLRLSCAASDRTFSSYAVGWFRQAPGQGREFVAAISWNGGRTLYTDSVTGRFTISRDNSKNTLYLQMNSLRAEDTAVYYCAARPAAPTRRLEYDYWGQGTLVTVSS

SEQ ID NO: 305 - protein, 122 aa, synthetic construct
QVQLVESGGGLVQPGGSLRLSCAASDRTFSSYAVGWFRQAPGQEREFVAAISWNGGRTLYTDSVTGRFTISRDNSKNTLYLQMNSLRAEDTAVYYCAARPAAPTRRLEYDYWGQGTLVTVSS

SEQ ID NO: 306 - protein, 122 aa, synthetic construct
QVQLVESGGGLVQPGGSLRLSCAASDRTFSSYAVGWFRQAPGKEREFVAAISWNGGRTLYTDSVTGRFTISRDNSKNTLYLQMNSLRAEDTAVYYCAARPAAPTRRLEYDYWGQGTLVTVSS

SEQ ID NO: 307 - protein, 8 aa, synthetic construct
DRTFSSYA

SEQ ID NO: 308 - protein, 5 aa, synthetic construct
SYAVG

SEQ ID NO: 309 - protein, 7 aa, synthetic construct
DRTFSSY

SEQ ID NO: 310 - protein, 8 aa, synthetic construct
ISWNGGRT

SEQ ID NO: 311 - protein, 17 aa, synthetic construct
AISWNGGRTLYTDSVTG

SEQ ID NO: 312 - protein, 6 aa, synthetic construct
SWNGGR

SEQ ID NO: 313 - protein, 15 aa, synthetic construct
AARPAAPTRRLEYDY

SEQ ID NO: 314 - protein, 13 aa, synthetic construct
RPAAPTRRLEYDY

SEQ ID NO: 315 - protein, 13 aa, synthetic construct
RPAAPTRRLEYDY

SEQ ID NO: 316 - protein, 353 aa, synthetic construct
QVQLVESGGGLVQPGGSLRLSCAASDRTFSSYAVGWFRQAPGQGLEAVAAISWNGGRTLYTDSVTGRFTISRDNSKNTLYLQMNSLRAEDTAVYYCAARPAAPTRRLEYDYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 317 - protein, 353 aa, synthetic construct
QVQLVESGGGLVQPGGSLRLSCAASDRTFSSYAVGWFRQAPGQGLEFVAAISWNGGRTLYTDSVTGRFTISRDNSKNTLYLQMNSLRAEDTAVYYCAARPAAPTRRLEYDYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 318 - protein, 353 aa, synthetic construct
QVQLVESGGGLVQPGGSLRLSCAASDRTFSSYAVGWFRQAPGQGREFVAAISWNGGRTLYTDSVTGRFTISRDNSKNTLYLQMNSLRAEDTAVYYCAARPAAPTRRLEYDYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 319 - protein, 353 aa, synthetic construct
QVQLVESGGGLVQPGGSLRLSCAASDRTFSSYAVGWFRQAPGQEREFVAAISWNGGRTLYTDSVTGRFTISRDNSKNTLYLQMNSLRAEDTAVYYCAARPAAPTRRLEYDYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 320 - protein, 353 aa, synthetic construct
QVQLVESGGGLVQPGGSLRLSCAASDRTFSSYAVGWFRQAPGKEREFVAAISWNGGRTLYTDSVTGRFTISRDNSKNTLYLQMNSLRAEDTAVYYCAARPAAPTRRLEYDYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 321-400 - skipped (80 entries)

SEQ ID NO: 401 - protein, 115 aa, synthetic construct
QVQLVESGGGMVQPGGSLRLSCAASGITFSMYSMSWYRQPPGKQRELVAATTTFGSTNYADSVKGRFTISRDNAKNTVYLQMNSLKPEDTAVYLCNARFTSEEYWGQGTQVTVSS

SEQ ID NO: 402 - protein, 115 aa, synthetic construct
QVQLVESGGGLVQPGGSLRLSCAASGITFSMYSMSWFRQAPGQGLEAVAATTTFGSTNYADSVKGRFTISRDNSKNTLYLQMNSLRAEDTAVYYCAARFTSEEYWGQGTLVTVSS

SEQ ID NO: 403 - protein, 115 aa, synthetic construct
QVQLVESGGGLVQPGGSLRLSCAASGITFSMYSMSWFRQAPGQGLELVAATTTFGSTNYADSVKGRFTISRDNSKNTLYLQMNSLRAEDTAVYYCAARFTSEEYWGQGTLVTVSS

SEQ ID NO: 404 - protein, 115 aa, synthetic construct
QVQLVESGGGLVQPGGSLRLSCAASGITFSMYSMSWYRQAPGQGLELVAATTTFGSTNYADSVKGRFTISRDNSKNTLYLQMNSLRAEDTAVYYCAARFTSEEYWGQGTLVTVSS

SEQ ID NO: 405 - protein, 115 aa, synthetic construct
QVQLVESGGGLVQPGGSLRLSCAASGITFSMYSMSWYRQAPGQQRELVAATTTFGSTNYADSVKGRFTISRDNSKNTLYLQMNSLRAEDTAVYYCAARFTSEEYWGQGTLVTVSS

SEQ ID NO: 406 - protein, 115 aa, synthetic construct
QVQLVESGGGLVQPGGSLRLSCAASGITFSMYSMSWYRQPPGKQRELVAATTTFGSTNYADSVKGRFTISRDNSKNTLYLQMNSLRAEDTAVYYCAARFTSEEYWGQGTLVTVSS

SEQ ID NO: 407 - protein, 8 aa, synthetic construct
GITFSMYS

SEQ ID NO: 408 - protein, 5 aa, synthetic construct
MYSMS

SEQ ID NO: 409 - protein, 7 aa, synthetic construct
GITFSMY

SEQ ID NO: 410 - protein, 7 aa, synthetic construct
TTTFGST

SEQ ID NO: 411 - protein, 16 aa, synthetic construct
ATTTFGSTNYADSVKG

SEQ ID NO: 412 - protein, 5 aa, synthetic construct
TTFGS

SEQ ID NO: 413 - protein, 9 aa, synthetic construct
AARFTSEEY

SEQ ID NO: 414 - protein, 7 aa, synthetic construct
RFTSEEY

SEQ ID NO: 415 - protein, 7 aa, synthetic construct
RFTSEEY

SEQ ID NO: 416 - protein, 346 aa, synthetic construct
QVQLVESGGGLVQPGGSLRLSCAASGITFSMYSMSWFRQAPGQGLEAVAATTTFGSTNYADSVKGRFTISRDNSKNTLYLQMNSLRAEDTAVYYCAARFTSEEYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 417 - protein, 346 aa, synthetic construct
QVQLVESGGGLVQPGGSLRLSCAASGITFSMYSMSWFRQAPGQGLELVAATTTFGSTNYADSVKGRFTISRDNSKNTLYLQMNSLRAEDTAVYYCAARFTSEEYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 418 - protein, 346 aa, synthetic construct
QVQLVESGGGLVQPGGSLRLSCAASGITFSMYSMSWYRQAPGQGLELVAATTTFGSTNYADSVKGRFTISRDNSKNTLYLQMNSLRAEDTAVYYCAARFTSEEYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 419 - protein, 346 aa, synthetic construct
QVQLVESGGGLVQPGGSLRLSCAASGITFSMYSMSWYRQAPGQQRELVAATTTFGSTNYADSVKGRFTISRDNSKNTLYLQMNSLRAEDTAVYYCAARFTSEEYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 420 - protein, 346 aa, synthetic construct
QVQLVESGGGLVQPGGSLRLSCAASGITFSMYSMSWYRQPPGKQRELVAATTTFGSTNYADSVKGRFTISRDNSKNTLYLQMNSLRAEDTAVYYCAARFTSEEYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 421-500 - skipped (80 entries)

SEQ ID NO: 501 - protein, 116 aa, synthetic construct
QVQVVESGGGLVQPGGSLTLSCAASGFDFSTYTVNWYRQAPGKEREKVARISSTGTTTNYANSAKGRFTLSRDNAKNRVYLQMNNLKPEDTAVYFCNFERFDSNYWGQGTQVTVSS

SEQ ID NO: 502 - protein, 116 aa, synthetic construct
QVQLVESGGGLVQPGGSLRLSCAASGFDFSTYTVNWFRQAPGQGLEAVARISSTGTTTNYANSAKGRFTISRDNSKNTLYLQMNSLRAEDTAVYYCAAERFDSNYWGQGTLVTVSS

SEQ ID NO: 503 - protein, 116 aa, synthetic construct
QVQLVESGGGLVQPGGSLRLSCAASGFDFSTYTVNWFRQAPGQGLEKVARISSTGTTTNYANSAKGRFTISRDNSKNTLYLQMNSLRAEDTAVYYCAAERFDSNYWGQGTLVTVSS

SEQ ID NO: 504 - protein, 116 aa, synthetic construct
QVQLVESGGGLVQPGGSLRLSCAASGFDFSTYTVNWYRQAPGQGLEKVARISSTGTTTNYANSAKGRFTISRDNSKNTLYLQMNSLRAEDTAVYYCAAERFDSNYWGQGTLVTVSS

SEQ ID NO: 505 - protein, 116 aa, synthetic construct
QVQLVESGGGLVQPGGSLRLSCAASGFDFSTYTVNWYRQAPGQEREKVARISSTGTTTNYANSAKGRFTISRDNSKNTLYLQMNSLRAEDTAVYYCAAERFDSNYWGQGTLVTVSS

SEQ ID NO: 506 - protein, 116 aa, synthetic construct
QVQLVESGGGLVQPGGSLRLSCAASGFDFSTYTVNWYRQAPGKEREKVARISSTGTTTNYANSAKGRFTISRDNSKNTLYLQMNSLRAEDTAVYYCAAERFDSNYWGQGTLVTVSS

SEQ ID NO: 507 - protein, 8 aa, synthetic construct
GFDFSTYT

SEQ ID NO: 508 - protein, 5 aa, synthetic construct
TYTVN

SEQ ID NO: 509 - protein, 7 aa, synthetic construct
GFDFSTY

SEQ ID NO: 510 - protein, 8 aa, synthetic construct
ISSTGTTT

SEQ ID NO: 511 - protein, 17 aa, synthetic construct
RISSTGTTTNYANSAKG

SEQ ID NO: 512 - protein, 6 aa, synthetic construct
SSTGTT

SEQ ID NO: 513 - protein, 9 aa, synthetic construct
AAERFDSNY

SEQ ID NO: 514 - protein, 9 aa, synthetic construct
AAERFDSNY

SEQ ID NO: 515 - protein, 7 aa, synthetic construct
ERFDSNY

SEQ ID NO: 516 - protein, 347 aa, synthetic construct
QVQLVESGGGLVQPGGSLRLSCAASGFDFSTYTVNWFRQAPGQGLEAVARISSTGTTTNYANSAKGRFTISRDNSKNTLYLQMNSLRAEDTAVYYCAAERFDSNYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 517 - protein, 347 aa, synthetic construct
QVQLVESGGGLVQPGGSLRLSCAASGFDFSTYTVNWFRQAPGQGLEKVARISSTGTTTNYANSAKGRFTISRDNSKNTLYLQMNSLRAEDTAVYYCAAERFDSNYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 518 - protein, 347 aa, synthetic construct
QVQLVESGGGLVQPGGSLRLSCAASGFDFSTYTVNWYRQAPGQGLEKVARISSTGTTTNYANSAKGRFTISRDNSKNTLYLQMNSLRAEDTAVYYCAAERFDSNYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 519 - protein, 347 aa, synthetic construct
QVQLVESGGGLVQPGGSLRLSCAASGFDFSTYTVNWYRQAPGQEREKVARISSTGTTTNYANSAKGRFTISRDNSKNTLYLQMNSLRAEDTAVYYCAAERFDSNYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 520 - protein, 347 aa, synthetic construct
QVQLVESGGGLVQPGGSLRLSCAASGFDFSTYTVNWYRQAPGKEREKVARISSTGTTTNYANSAKGRFTISRDNSKNTLYLQMNSLRAEDTAVYYCAAERFDSNYWGQGTLVTVSSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 521 - protein, 15 aa, synthetic construct
GGGGSGGGGSGGGGS

SEQ ID NO: 522 - protein, 12 aa, synthetic construct
GGSGGSGGSGGS

SEQ ID NO: 523 - protein, 9 aa, synthetic construct
GGSGGSGGS

(19) United States
(12) Patent Application Publication
(10) Pub. No.: US 2026/0137822 A1
(43) Pub. Date: May 21, 2026

(54) MULTIVALENT IMMUNOCONJUGATES FOR TARGETED RADIOISOTOPE THERAPY

(71) Applicant: ABDERA THERAPEUTICS INC., South San Francisco, CA (US)

(72) Inventors: Michael J. ABRAMS, Custer, WA (US); Emma Jane CUMMINS, Vancouver, BC (CA); Adam Daniel JUDGE, Bainbridge Island, WA (US); Alexander Laurence MANDEL, Vancouver, BC (CA); Raja Solomon VISWAS, Richmond, BC (CA)

(21) Appl. No.: 19/104,553

(22) PCT Filed: Aug. 21, 2023
(86) PCT No.: PCT/US2023/072588

§ 371 Date: Feb. 18, 2025

Related U.S. Application Data
(60) Provisional application No. 63/373,186, filed on Aug. 22, 2022.

(51) Int. Cl. A61K 51/10; A61P 35/00; C07K 16/30; C07K 16/46; C12N 15/11; C12N 15/63

(52) U.S. Cl. CPC A61K 51/1045; A61K 51/1084; A61P 35/00; C07K 16/30; C07K 16/46; C12N 15/11; C12N 15/63; C07K 2317/35; C07K 2317/52; C07K 2317/569; C07K 2317/71; C07K 2317/94

(57) ABSTRACT

Described herein are immunoconjugates comprising a multivalent antibody and a chelating agent, wherein the multivalent antibody comprises a polypeptide comprising: a first antigen-binding domain; and a second antigen-binding domain.

### CROSS-REFERENCE

This application claims the benefit of U.S. Provisional Application Ser. No. 63/373,186 filed on Aug. 22, 2022, which is incorporated by reference herein in its entirety.

### BACKGROUND

The specificity of antibodies, such as IgGs, to their antigens makes antibodies a premier targeting platform for therapeutics; however, the typical serum half-life of at least three weeks for an IgG is disadvantageous for the delivery of radioisotopes including alpha-emitting isotopes such as Ac-225 and beta-emitting isotopes such as Lu-177 and Y-90, in particular due to prolonged exposure and chronic off-target toxicities

225-Ac is among the most cytotoxic of the a-emitting radioisotopes, and a single decay event can effectively destroy a cancer cell by causing double-strand DNA breaks and subsequent cell death. The potency of a-emitting radioisotopes makes them attractive as cell killing agents, capable of overcoming the acquired resistance observed in response to other therapies.

### SUMMARY

Provided herein are immunoconjugates (e.g., radiolabeled immunoconjugates) that comprise a multivalent (e.g., tetravalent) antibody and are useful for treating cancer. The immunoconjugates described herein are advantageous in that the immunoconjugates are able to achieve higher order avidity interactions with a target while having a molecular weigh less than a conventional antibody molecule (e.g., less than 150,000 Daltons), and while showing improved safety profiles (e.g., reduced serum half-life).

Described herein in one aspect is an immunoconjugate comprising a multivalent antibody and a chelating agent, wherein the multivalent antibody comprises a polypeptide comprising: (a) a first antigen-binding domain; and (b) a second antigen-binding domain. In certain embodiments, the polypeptide further comprises an Fc domain.

Described herein in another aspect is an immunoconjugate comprising a multivalent antibody and a chelating agent, wherein the multivalent antibody comprises a polypeptide having the structure of Formula I:

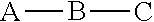

wherein: A comprises a first antigen-binding domain; B comprises a second antigen-binding domain; and C comprises an Fc domain.

Described herein in another aspect is an immunoconjugate comprising a multivalent antibody and a chelating agent, wherein the multivalent antibody comprises a polypeptide having the structure of Formula II: A-C-B; wherein: A comprises a first antigen-binding domain; B comprises a second antigen-binding domain; and C comprises an Fc domain.

Described herein in another aspect is an immunoconjugate comprising a multivalent antibody, a chelating agent, and a radioisotope, wherein the multivalent antibody comprises homodimer of a polypeptide having the structure of Formula I: A-B-C; wherein:A comprises a first VHH domain; B comprises a second VHH domain; and C comprises an Fc domain, wherein the first VHH domain binds FOLR1 or DLL3; and wherein the second VHH domain binds FOLR1 or DLL3.

Described herein in another aspect is an immunoconjugate comprising a multivalent antibody, a chelating agent, and a radioisotope, wherein the multivalent antibody comprises homodimer of a polypeptide having the structure of Formula II: A-C-B; wherein: A comprises a first VHH domain; B comprises a second VHH domain; and C comprises an Fc domain, wherein the first VHH domain binds FOLR1 or DLL3; and wherein the second VHH domain binds FOLR1 or DLL3.

In certain embodiments, the first antigen-binding domain and the second antigen-binding domain each comprise an immunoglobulin single-chain variable domain polypeptide. In certain embodiments, the immunoglobulin single-chain variable domain polypeptide comprises a VHH. In certain embodiments, the Fc domain comprises a CH2 domain and CH3 domain. In certain embodiments, the Fc domain comprises a CH3 domain. In certain embodiments, the Fc domain comprises a CH2 domain. In certain embodiments, the Fc domain comprises an alteration to one or more amino acid residues that reduces an effector function of the Fc domain. In certain embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof. In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (11) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ppp), per EU numbering. In certain embodiments, the alteration to one or more amino acid residues that reduces the effector function of the immunoglobulin heavy chain constant region comprises L234A, L235E, G237A, A330S, and P331S per EU numbering. In certain embodiments, the Fc domain comprises an alteration to one or more amino acid residues that alters binding of the immunoconjugate to the neonatal Fc receptor (FcRn). In certain embodiments, the alteration to one or more amino acid residues that alters binding of the immunoconjugate to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, S254A, H310A, H435Q, Y436A and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, H310A, H435Q, and combinations thereof per EU numbering. In certain embodiments, the alteration to one or more amino acid residues that alters binding of the immunoconjugate to the neonatal Fc receptor (FcRn) comprises I253A per EU numbering. In certain embodiments, the alteration to one or more amino acid residues that alters binding of the immunoconjugate to the neonatal Fc receptor (FcRn) comprises H310A per EU numbering. In certain embodiments, the alteration to one or more amino acid residues that alters binding of the immunoconjugate to the neonatal Fc receptor (FcRn) comprises H435Q per EU numbering. In certain embodiments, the multivalent antibody comprises a homodimer of the polypeptide. In certain embodiments, the multivalent antibody comprises a molecular weigh less than about 110,000 Daltons. In certain embodiments, the multivalent antibody is a monospecific multivalent antibody. In certain embodiments, the multivalent antibody is a bispecific multivalent antibody. In certain embodiments, the first antigen-binding domain and the second antigen-binding domain binds FOLR1, DLL3, or HER2. In certain embodiments, the first antigen-binding domain binds FOLR1. In certain embodiments, (a) the first antigen-binding domain binds FOLR1; and (b) the second antigen-binding domain binds DLL3. In certain embodiments, the first antigen-binding domain comprises: a complementarity determining region (CDR) 1 comprising the amino acid sequence as set forth in SEQ ID NO: 1; a complementarity determining region (CDR) 2 comprising the amino acid sequence as set forth in SEQ ID NO: 2; and a complementarity determining region (CDR) 3 comprising the amino acid sequence as set forth in SEQ ID NO: 3. In certain embodiments, the second antigen-binding domain comprises: a complementarity determining region (CDR) 1 comprising the amino acid sequence as set forth in SEQ ID NO: 5; a complementarity determining region (CDR) 2 comprising the amino acid sequence as set forth in SEQ ID NO: 6; and a complementarity determining region (CDR) 3 comprising the amino acid sequence as set forth in SEQ ID NO: 7; a complementarity determining region (CDR) 1 comprising the amino acid sequence as set forth in SEQ ID NO: 107; a complementarity determining region (CDR) 2 comprising the amino acid sequence as set forth in SEQ ID NO: 110; and a complementarity determining region (CDR) 3 comprising the amino acid sequence as set forth in SEQ ID NO: 113; a complementarity determining region (CDR) 1 comprising the amino acid sequence as set forth in SEQ ID NO: 207; a complementarity determining region (CDR) 2 comprising the amino acid sequence as set forth in SEQ ID NO: 210; and a complementarity determining region (CDR) 3 comprising the amino acid sequence as set forth in SEQ ID NO: 213; a complementarity determining region (CDR) 1 comprising the amino acid sequence as set forth in SEQ ID NO: 307; a complementarity determining region (CDR) 2 comprising the amino acid sequence as set forth in SEQ ID NO: 310; and a complementarity determining region (CDR) 3 comprising the amino acid sequence as set forth in SEQ ID NO: 313; a complementarity determining region (CDR) 1 comprising the amino acid sequence as set forth in SEQ ID NO: 407; a complementarity determining region (CDR) 2 comprising the amino acid sequence as set forth in SEQ ID NO: 410; and a complementarity determining region (CDR) 3 comprising the amino acid sequence as set forth in SEQ ID NO: 413; or a complementarity determining region (CDR) 1 comprising the amino acid sequence as set forth in SEQ ID NO: 507; a complementarity determining region (CDR) 2 comprising the amino acid sequence as set forth in SEQ ID NO: 510; and a complementarity determining region (CDR) 3 comprising the amino acid sequence as set forth in SEQ ID NO: 513. In certain embodiments, the first antigen-binding domain comprises: a complementarity determining region (CDR) 1 comprising the amino acid sequence as set forth in SEQ ID NO: 1; a complementarity determining region (CDR) 2 comprising the amino acid sequence as set forth in SEQ ID NO: 2; and a complementarity determining region (CDR) 3 comprising the amino acid sequence as set forth in SEQ ID NO: 3, and wherein the second antigen-binding domain comprises: a complementarity determining region (CDR) 1 comprising the amino acid sequence as set forth in SEQ ID NO: 5; a complementarity determining region (CDR) 2 comprising the amino acid sequence as set forth in SEQ ID NO: 6; and a complementarity determining region (CDR) 3 comprising the amino acid sequence as set forth in SEQ ID NO: 7; a complementarity determining region (CDR) 1 comprising the amino acid sequence as set forth in SEQ ID NO: 107; a complementarity determining region (CDR) 2 comprising the amino acid sequence as set forth in SEQ ID NO: 110; and a complementarity determining region (CDR) 3 comprising the amino acid sequence as set forth in SEQ ID NO: 113; a complementarity determining region (CDR) 1 comprising the amino acid sequence as set forth in SEQ ID NO: 207; a complementarity determining region (CDR) 2 comprising the amino acid sequence as set forth in SEQ ID NO: 210; and a complementarity determining region (CDR) 3 comprising the amino acid sequence as set forth in SEQ ID NO: 213; a complementarity determining region (CDR) 1 comprising the amino acid sequence as set forth in SEQ ID NO: 307; a complementarity determining region (CDR) 2 comprising the amino acid sequence as set forth in SEQ ID NO: 310; and a complementarity determining region (CDR) 3 comprising the amino acid sequence as set forth in SEQ ID NO: 313; a complementarity determining region (CDR) 1 comprising the amino acid sequence as set forth in SEQ ID NO: 407; a complementarity determining region (CDR) 2 comprising the amino acid sequence as set forth in SEQ ID NO: 410; and a complementarity determining region (CDR) 3 comprising the amino acid sequence as set forth in SEQ ID NO: 413; or a complementarity determining region (CDR) 1 comprising the amino acid sequence as set forth in SEQ ID NO: 507; a complementarity determining region (CDR) 2 comprising the amino acid sequence as set forth in SEQ ID NO: 510; and a complementarity determining region (CDR) 3 comprising the amino acid sequence as set forth in SEQ ID NO: 513. In certain embodiments, the first antigen-binding domain or the second antigen-binding domain comprise an amino acid sequence at least about 85%, 90%, 95%, 97%, 98%, 99%, or 100% identical to that set forth in SEQ ID NO: 4. In certain embodiments, the first antigen-binding domain or the second antigen-binding domain comprise an amino acid sequence at least about 85%, 90%, 95%, 97%, 98%, 99%, or 100% identical to that set forth in SEQ ID NO: 8, 101-106, 201-206, 301-306, 401-406, or 501-506. In certain embodiments, the first antigen-binding domain comprises an amino acid sequence at least about 85%, 90%, 95%, 97%, 98%, 99%, or 100% identical to that set forth in SEQ ID NO: 4; and the second antigen-binding domain comprises an amino acid sequence at least about 85%, 90%, 95%, 97%, 98%, 99%, or 100% identical to that set forth in SEQ ID NO: 8, 101-106, 201-206, 301-306, 401-406, or 501-506. In certain embodiments, the chelating agent is a radioisotope chelating agent. In certain embodiments, the chelating agent is an alpha emitter chelating agent. In certain embodiments, the chelating agent is a beta- or gamma-emitter chelating agent. In certain embodiments, the chelating agent is selected from the list consisting of: DOTA, DO3A, DOTAGA, DOTAGA anhydride, Py4Pa, Py4Pa-NCS, Crown, Macropa, Macropa-NCS, HEHA, CHXoctapa, Bispa, Noneunpa, and combinations thereof. In certain embodiments, the chelating agent is selected from the list consisting of: DOTMA, DOTPA, DO3AM-acetic acid, DOTP, DOTMP, DOTA-4AMP, CB-TE2A, NOTA, NOTP, TETPA, TETA, PEPA, H4Octapa, H2Dedpa, DO2P, EDTA, DTPA-BMA, 3, 2, 3-LI(HOPO), 3, 2-HOPO, Neunpa, Neunpa-NCS, Octapa, PyPa, Porphyrin, Deferoxamine, DFO*, and combinations thereof. In certain embodiments, the chelating agent is DOTA. In certain embodiments, the chelating agent is DOTAGA. In certain embodiments, the chelating agent is Py4Pa. In certain embodiments, the chelating agent is directly coupled to the antigen binding region and/or the Fc domain. In certain embodiments, the chelating agent is coupled to the antigen binding region and/or Fc domain by a linker. In certain embodiments, the chelating agent is a linker-chelator selected from the list consisting of. TFP-Ad-PEG5-DOTAGA, p-SCN-Bn-DOTA, p-SCN-Ph-Et-Py4Pa, and TFP-Ad-PEG5-Ac-Py4Pa. In certain embodiments, the immunoconjugate further comprises a radioisotope. In certain embodiments, the radioisotope is an alpha emitter. In certain embodiments, the radioisotope is an alpha emitter selected from the list consisting of 225-Ac, 223-Ra, 224-Ra, 227-Th, 212-Pb, 212-Bi, and 213-Bi. In certain embodiments, the radioisotope is 225-Ac. In certain embodiments, the radioisotope is a beta emitter. In certain embodiments, the radioisotope is a beta emitter selected from 177-Lu, 90-Y, 67-Cu, and 153-Sm.

A method of killing a tumor cell or a cancer cell, the method comprising: contacting the tumor cell or the cancer cell with an immunoconjugate of this disclosure, thereby killing the tumor cell or cancer cell. In certain embodiments, the tumor cell is a solid tumor cell. In certain embodiments, the tumor cell or cancer cell express FOLR1, DLL3, or both.

A method of treating a cancer or a tumor in an individual comprising administering to the individual the immunoconjugate herein, thereby treating the cancer or the tumor. In certain embodiments, the individual is a human individual. In certain embodiments, the cancer or the tumor is a solid cancer or tumor. In certain embodiments, the cancer or the tumor comprises lung cancer, breast cancer, ovarian cancer, or a neuroendocrine cancer. In certain embodiments, the method comprises administering from 0.5 μCi to 30.0 μCi per kilogram to the individual. In certain embodiments, the method comprises administering from 10 m Ci to 75 mCi per meter squared of body area to the individual. In certain embodiments, the cancer or tumor expresses an antigen specifically bound by the immunoconjugate. In certain embodiments, the immunoconjugates is for use in a method of treating a cancer or a tumor in an individual.

Also described herein is a method of delivering a radioisotope to a cancer cell or a tumor cell in an individual comprising administering to the individual the immunoconjugate, thereby delivering the radioisotope to the cancer cell or the tumor cell. In certain embodiments, the individual is a human individual. In certain embodiments, the cancer cell or tumor cell comprises a lung cancer cell, a breast cancer cell, an ovarian cancer cell, or a neuroendocrine cancer cell. In certain embodiments, the cancer cell or tumor cell expresses an antigen specifically bound by the immunoconjugate.

Also described herein is a method of imaging a tumor in an individual comprising administering to the individual the immunoconjugate. In certain embodiments, the individual is a human individual. In certain embodiments, the tumor comprises lung cancer, breast cancer, ovarian cancer, or a neuroendocrine cancer. In certain embodiments, the tumor expresses an antigen specifically bound by the immunoconjugate.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B show binding of anti-HER2 and anti-DLL3 VHH-Fc constructs.

FIGS. 2A, 2B, and 2C show binding of anti-HER2 and anti-DLL3 VHH-Fc constructs to cells expressing HER2 and/or DLL3.

FIGS. 3A and 3B show internalization of anti-HER2 and anti-DLL3 VHH-Fc constructs in cells expressing HER2 and DLL3.

FIG. 4 shows self-interaction data for anti-HER2 and anti-DLL3 VHH-Fc constructs.

FIG. 5 shows a diagram for chemical synthesis of linker molecules.

FIG. 6 shows a diagram for chemical synthesis of linker molecules.

FIGS. 7A, 7B, and 7C shows the immunoreactive fraction of different VHH-Fc constructs.

FIG. 8 shows a comparison of imaging with 111In labeled VHH-Fc compared to biodistribution of 225Ac labeled VHH-Fc.

FIGS. 9A, 9B, 9C, and 9D show biodistribution over time for labeled anti-HER2 VHH-Fc constructs.

FIGS. 10A, 10B and 10C show tumor:non-tumor tissue ratios for labeled anti-HER2 VHH-Fc constructs.

FIG. 11 shows biodistribution for labeled anti-HER2 VHH-Fc constructs.

FIG. 12 shows whole body clearance of VHH-Fc (H101) and VHH-Fc variants (H105, H107, and H108) labeled with 111In.

FIG. 13 shows biodistribution over time for labeled anti-DLL3 VHH-Fc constructs.

FIG. 14 shows biodistribution for labeled anti-DLL3 VHH-Fc constructs.

FIGS. 15A and 15B show biodistribution for 225Ac labeled anti-HER2 (15A) and anti-DLL3 (15B) VHH-Fc constructs.

FIGS. 16A, 16B, and 16C show the results of a toxicity study carried out with 225Ac labeled anti-HER2 VHH-Fc constructs.

FIG. 17 shows the immunoreactive fraction of different anti-DDL3 VHH-Fc constructs loaded with 177Lu.

FIG. 18 shows the chemical Structures of certain linker chelators described herein.

FIGS. 19A and 19B show a schematic of multivalent antibody formats.

FIG. 20 shows FOLR1 and DLL3 monospecific and bispecific multivalent formats.

FIG. 21 shows binding of multivalent tetramer constructs to cells expressing FOLR1.

### DETAILED DESCRIPTION

Provided herein are immunoconjugates (e.g., radiolabeled immunoconjugates) that comprise a multivalent (e.g., tetravalent) antibody and are useful for treating cancer. In certain instances, the immunoconjugates described herein are advantageous in that they achieve higher order avidity interactions with a target while having a molecular weigh less than a conventional antibody molecule (e.g., less than 150,000 Daltons). Furthermore, in such instances, the immunoconjugates comprise elements of conventional antibodies such as an Fc domain or Fc variant domain yet can achieve the higher order avidity interactions at lower molecular weights. In some embodiments described herein, the immunoconjugates utilize Fc mutations that reduce serum half-life and effector celssl functions. Although reducing reduce serum half-life or effector cell function is generally not associated with improved antibody efficacy, the immunoconjugates described herein can achieve improved safety while being able effectively bind and kill target tumor cells (e.g., within a tumor microenvironment).

### Immunoconjugates

Provided herein are immunoconjugates (e.g., a radiolabeled immunoconjugate) comprising a multivalent (e.g., tetravalent or greater) polypeptide (e.g., a multivalent antibody or antibody-derived polypeptide). In some embodiments, the multivalent antibodys are tetravalent. In certain embodiments, the multivalent antibodys are monospecific (e.g., binding only FOLR1 or DLL3). In certain embodiments, the multivalent antibodys are bispecific (e.g., binding both FOLR1 and DLL3). In some embodiments, the multivalent antibody of the immunoconjugate comprises a molecular weigh less than 150,000 Daltons. In certain embodiments, the multivalent antibody of the immunoconjugate comprises a molecular weight less than 110,000 Daltons.

In some embodiments, provided are immunoconjugates (e.g., radio-immunoconjugates) (e.g., having a chelating agent and a radionuclide) comprising a multivalent antibody, wherein the multivalent antibody comprises a polypeptide comprising: a first antigen-binding domain; and a second antigen-binding domain. In certain embodiments, the polypeptide further comprises an Fc domain.

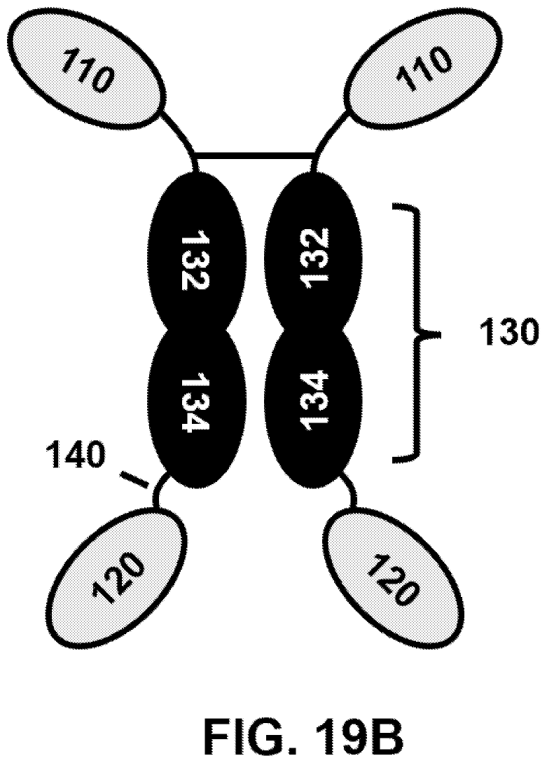

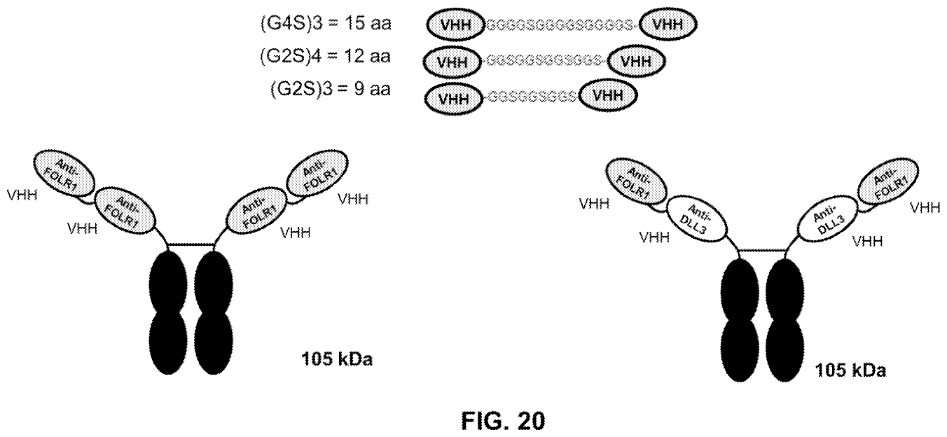

FIGS. 19A-B show exemplary multivalent antibody formats described herein. 110 depicts a first antigen-binding domain (e.g., an immunoglobulin single-chain domain). 120 depicts a second antigen-binding domain (e.g., an immunoglobulin single-chain domain). 130 depicts an Fc domain comprising a CH2-CH3 (132 and 134, respectively). 140 depicts an optional linker polypeptide.

In some embodiments, provided are immunoconjugates (e.g., radio-immunoconjugates) comprising a multivalent antibody and a chelating agent, wherein the multivalent antibody comprises a polypeptide having the structure of Formula I:

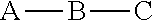

wherein: A comprises a first antigen-binding domain; B comprises a second antigen-binding domain; and C comprises an Fc domain.

In some embodiments, provided are immunoconjugates (e.g., radio-immunoconjugates) comprising a multivalent antibody and a chelating agent, wherein the multivalent antibody comprises a polypeptide having the structure of Formula II:

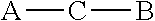

wherein: A comprises a first antigen-binding domain; B comprises a second antigen-binding domain; and C comprises an Fc domain.

In some embodiments, provided are immunoconjugates (e.g., radio-immunoconjugates) comprising a multivalent antibody and a chelating agent, wherein the multivalent antibody comprises a polypeptide having the structure of Formula III:

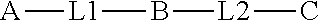

wherein: A comprises a first antigen-binding domain; B comprises a second antigen-binding domain; C comprises an Fc domain; L1 is a polypeptide linker (e.g., a poly GS linker); and L2 is a polypeptide linker (e.g., identical to or different from L1).

In some embodiments, provided are immunoconjugates (e.g., radio-immunoconjugates) comprising a multivalent antibody and a chelating agent, wherein the multivalent antibody comprises a polypeptide having the structure of Formula IV:

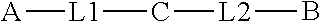

wherein: A comprises a first antigen-binding domain; B comprises a second antigen-binding domain; C comprises an Fc domain; L1 is a polypeptide linker (e.g., a poly GS linker); and L2 is a polypeptide linker (e.g., identical to or different from L1).

In certain embodiments, the multivalent antibody comprises a homodimer of the polypeptide (e.g., mediated through Fc domain dimerization). In certain embodiments, the multivalent antibody comprises a molecular weight less than 150,000 Daltons. In certain embodiments, the multivalent antibody comprises a molecular weight less than 140,000 Daltons. In certain embodiments, the multivalent antibody comprises a molecular weight less than 130,000 Daltons. In certain embodiments, the multivalent antibody comprises a molecular weight less than 120,000 Daltons. In certain embodiments, the multivalent antibody comprises a molecular weight less than 110,000 Daltons. In certain embodiments, an antigen-binding domain comprises molecular weight of about 50,000 Daltons to about 110,000 Daltons. In certain embodiments, the multivalent antibody comprises a molecular weight less than 110,000 Daltons. In certain embodiments, an antigen-binding domain comprises molecular weight of about 75,000 Daltons to about 110,000 Daltons. In certain embodiments, an antigen-binding domain comprises molecular weight of about 50,000 Daltons to about 105,000 Daltons. In certain embodiments, the multivalent antibody comprises a molecular weight less than 110,000 Daltons. In certain embodiments, an antigen-binding domain comprises molecular weight of about 75,000 Daltons to about 105,000 Daltons.

In certain embodiments, the first antigen-binding domain and second antigen-binding domain each comprise a single-chain variable domain polypeptide selected from the group consisting of a scFv, VH, VL, VHH, and VNAR. In certain embodiments, the first antigen-binding domain and second antigen-binding domain each comprise an immunoglobulin single-chain variable domain polypeptide. In certain embodiments, the immunoglobulin single-chain variable domain polypeptide is selected from the group consisting of a VH, VL, and VHH. In certain embodiments, the first antigen-binding domain and second antigen-binding domain each comprise a VHH (e.g., a first VHH domain and a second VHH domain).

In certain embodiments, the Fc domain comprises an alteration to one or more amino acid residues that modulates (e.g., reduces, inhibits, decreases, prevents, etc.) an effector function of the Fc domain. In certain embodiments, the Fc domain comprises an alteration to one or more amino acid residues that alters (e.g., reduces, inhibits, decreases, prevents, etc.) serum half-life of the immunoconjugate. In certain embodiments, the Fc domain comprises an alteration to one or more amino acid residues that alters (e.g., reduces, inhibits, decreases, prevents, etc.) binding of the immunoconjugate to the neonatal Fc receptor (FcRn). In certain embodiments, the Fc domain comprises an alteration to one or more amino acid residues that modulates (e.g., reduces, inhibits, decreases, prevents, etc.) both (i) an effector function of the Fc domain (e.g., ADCC and/or CDC) and (ii) binding of the immunoconjugate to the neonatal Fc receptor (FcRn).

In certain embodiments, the multivalent antibody is a monospecific (e.g., binding only FOLR1 or DLL3). In certain embodiments, the first antigen-binding domain and the second antigen-binding domain bind FOLR1. In certain embodiments, the first antigen-binding domain and the second antigen-binding domain bind DLL3.

In certain embodiments, the multivalent antibody is a bispecific (e.g., binding FOLR1 and DLL3). In certain embodiments, the first antigen-binding domain binds FOLR1; and the second antigen-binding domain binds DLL3. In certain embodiments, the first antigen-binding domain binds DLL3; and the second antigen-binding domain binds FOLR1.

In certain embodiments, the immunoconjugate comprises a chelating agent. In certain embodiments, the chelating agent is a radionuclide chelating agent. In certain embodiments, the chelating agent is directly coupled to the antigen binding region and/or the Fc domain. In certain embodiments, the chelating agent is indirectly coupled to the antigen binding region and/or the Fc domain.

In certain embodiments, the immunoconjugate is a radio-immunoconjugate, comprising a radionuclide. In certain embodiments, the radionuclide is an alpha-emitter. In certain embodiments, the radionuclide is a beta emitter. In certain embodiments, the radionuclide is an alpha emitter selected from the list consisting of 225-Ac, 223-Ra, 224-Ra, 227-Th, 212-Pb, 212-Bi, and 213-Bi.

A “immunoconjugate” refers to and encompasses a molecular complex comprising an at least one antigen binding region derived from a multivalent antibody (e.g., variable regions or complementarity determining regions) further coupled to at least one non-antibody derived molecule, such as a chelator or cytotoxic agent. Non-antibody derived molecules may for example be conjugated to one or more lysine or cysteine resides of the antigen binding region or to a constant region coupled (by peptide linkage or otherwise) to the antigen binding region. In some embodiments, the immunoconjugate further comprises a chelating agent (interchangeably, “chelator”). In some embodiments, an immunoconjugate comprises an antibody construct of the invention linked directly or indirectly to a cytotoxic agent or radioisotope.

### Antigen-Binding Domains

A “variable region” or “variable domain” refers to and encompasses the domain of an antibody heavy or light chain or immunoglobulin single-chain variable domain (e.g., VHH) antibody that is involved in binding the antibody to an antigen. The variable domains of the heavy chain, light chain (VH and VL, respectively), or VHH of a native antibody generally have similar structures, with each domain comprising four conserved framework regions (FRs) and three hypervariable regions (HVRs). (See, e.g., Kindt et al. Kuby Immunology, 6th ed., W.H. Freeman and Co. (2007). A single VH or VL domain may be sufficient to confer antigen-binding specificity. A single VHH is sufficient to confer antigen-binding specificity.

A “immunoglobulin single-chain variable domain” or “immunoglobulin single-chain variable domain antibody” or “immunoglobulin single variable domain” or “single chain antibpody” or “VHH”, interchangeably used herein, refers to and encompasses immunoglobulin molecules wherein the antigen binding site is present on, and formed by, a single immunoglobulin domain (e.g., variable domain).

Typically, in conventional immunoglobulins, a heavy chain variable domain (VH) and a light chain variable domain (VL) interact to form an antigen binding site, i.e., providing a total of 6 CDRs for antigen binding site formation. In this case, the complementarity determining regions (CDRs) of both VH and VL can contribute to the antigen. The antigen-binding domain of a conventional antibody (such as an IgG, IgM, IgA, IgD or IgE molecule having a cognate VL and VH), of a Fab fragment, a F(ab′)2 fragment, an Fv fragment such as a disulphide linked Fv, a scFv fragment, or a diabody derived from such conventional 4-chain antibody, is distinct from a single chain variable domain antibody.

A VH, VL, or VHH region can be subdivided into regions of hypervariability, termed “complementarity determining regions” (CDR), interspersed with regions that are more conserved, termed “framework regions” (FR or FW).

The extent of the framework region and CDRs can defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme). Methods encompassed and included herein Chothia, AbM, Kabat, Contact, and/or IMGT.

A “complementarity determining region” or “CDR” refers to and encompasses the sequences of amino acids within antibody variable regions which confer antigen specificity and binding affinity. In general, there are three CDRs in each heavy chain variable region (HCDR1, HCDR2, and HCDR3) and three CDRs in each light chain variable region (LCDR1, LCDR2, and LCDR3). An immunoglobulin single-chain variable domain antibody comprises 3 CDRs (CDR1, CDR2, and CDR3).

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). An immunoglobulin single-chain variable domain can for example be a heavy chain ISVD, such as a VH, VHH, including a camelized VH or humanized VHH. Preferably, it is a VHH, including a camelized VH or humanized VHH.

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “VHH domain” and “immunoglobulin single-chain variable domain” is used to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans (Reviews in Molecular Biotechnology 74: 277-302, 2001).

A “humanized VHH” comprises an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g. indicated above). This can be performed in a manner known per se, which will be clear to the skilled person, for example on the basis of the further description herein and the prior art (e.g. WO 2008/020079). Again, it should be noted that such humanized VHHS can be obtained in any suitable manner known per se and thus are not strictly limited to polypeptides that have been obtained using a polypeptide that comprises a naturally occurring VHH domain as a starting material.

Affinity encompasses and/or refers to the strength of the sum total of noncovalent interactions between a single binding site of a molecule (e.g., an antibody) and its binding partner (e.g., an antigen). Unless indicated otherwise, as used herein, binding affinity encompasses and refers to intrinsic binding affinity which reflects a 1:1 interaction between members of a binding pair (e.g., antibody and antigen). The affinity of a molecule X for its partner Y can be represented by the dissociation constant (KD). Affinity can be measured by common methods known in the art, including those described herein. Specific illustrative and exemplary embodiments for measuring binding affinity are described throughout.

An affinity matured antibody encompasses and/or refers to an antibody with one or more alterations in one or more hypervariable regions (HVRs), compared to a parent antibody which does not possess such alterations, such alterations resulting in an improvement in the affinity of the antibody for antigen.

Binding and a determination of binding can be readily determined by methods known within the art (e.g., ELISA, surface plasmon resonance, bio-layer interferometry, isothermal calorimetry, etc.). In some embodiments, binding is determined by ELISA. In some embodiments, binding comprising a KD less than, e.g., 10\hat{ }-5 M (10 uM) as measured by surface plasmon resonance, bio-layer interferometry, or isothermal calorimetry. In some embodiments, binding comprising a KD less than, e.g., 10\hat{ }-6 M (1 uM) surface plasmon resonance, bio-layer interferometry, or isothermal calorimetry. In some embodiments, binding comprising a KD less than, e.g., 10\hat{ }-7 M (100 nM) surface plasmon resonance, bio-layer interferometry, or isothermal calorimetry.

In certain embodiments, the antibody comprises one or more naturally occurring amino acids. In certain embodiments, the antibody consists of naturally occurring amino acids. As used herein, naturally occurring amino acids include and/or refer to amino acids which are found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the antibody comprises a variant sequence of the antibody. In certain instances, amino acid substitutions can be made in the sequence of any of the antibodies described herein, without necessarily decreasing or ablating its activity (as measured by, e.g., the binding or functional assays described herein). Accordingly, in some embodiments, the variant sequence comprises one or more amino acid substitutions (e.g., within the variable region or within one or more CDRs). In some embodiments, the variant sequence comprises one or more substitutions in one or more CDRs. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In certain embodiments, the antibody comprises one or more non-natural amino acids. In certain embodiments, the antibody consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring antibody sequence. A non-natural amino acid residue also can be incorporated such that a desired functionality is imparted to the apelin molecule, for example, the ability to link a functional moiety (e.g., PEG).

A stable formulation refers to and/or encompasses a formulation wherein the protein (e.g., antibody) therein essentially retains its physical stability and/or chemical stability and/or biological activity upon storage at an intended storage temperature, e.g., 2-8° C. In some embodiments, the formulation essentially retains its physical and chemical stability, as well as its biological activity upon storage. A storage period can be selected based on the intended shelf-life of the formulation. Furthermore, the formulation is stable following freezing (to, e.g., −20° C.) and thawing of the formulation, for example following 1 or more cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; SDS-PAGE analysis to compare reduced and intact antibody; evaluating biological activity or antigen binding function of the antibody; and the methods described herein. Instability can involve any one or more of: aggregation, deamidation (e.g., Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), etc.

A pharmaceutically acceptable carrier encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

A polypeptide or protein are used interchangeably, and encompass and/or refer to a polymer of amino acid residues and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

The determination of percent identity or percent similarity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Alternatively, sequence alignment may be carried out using the CLUSTAL algorithm (e.g., as provided in the program Clustal-omega), as described by Higgins et al., 1996, Methods Enzymol. 266:383-402.

As used herein the term individual, patient, or subject includes and/or refers to individuals diagnosed with, suspected of being afflicted with, or at-risk of developing at least one disease, condition, or status for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antibody of the disclosure; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

As used herein, treatment or treating include and/or refer to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated.

Typically, the generation of immunoglobulins involves the immunization of experimental animals, fusion of immunoglobulin producing cells to create hybridomas and screening for the desired specificities. Alternatively, immunoglobulins can be generated by screening of naive or synthetic libraries e.g. by phage display. The generation of immunoglobulin sequences, such as VHHs and immunoglobulin single-chain variable domain, has been described in various publications, among which WO 94/04678, Hamers-Casterman et al. 1993 and Muyldermans et al. 2001 (Reviews in Molecular Biotechnology 74: 277-302, 2001) can be exemplified. In these methods, camelids are immunized with the target antigen in order to induce an immune response against said target antigen. The repertoire of Nanobodies obtained from said immunization is further screened for Nanobodies that bind the target antigen. In these instances, the generation of antibodies requires purified antigen for immunization and/or screening. Antigens can be purified from natural sources, or during recombinant production.

The multivalent antibodies described herein comprise an antigen-binding domain. In certain instances, independent any antigen-binding domain having a molecular weigh less than about 25,000 Daltons (e.g., a VHH having a molecular weight of about 15,000 Daltons) can be used herein, wherein the multivalent antibody of the immunoconjugate totals a molecular weight of less than 150,000 Daltons. In certain embodiments, an antigen-binding domain comprises molecular weight of less than about 25,000 Daltons. In certain embodiments, an antigen-binding domain comprises molecular weight of less than about 20,000 Daltons. In certain embodiments, an antigen-binding domain comprises molecular weight of less than about 19,000 Daltons. In certain embodiments, an antigen-binding domain comprises molecular weight of less than about 18,000 Daltons. In certain embodiments, an antigen-binding domain comprises molecular weight of less than about 17,000 Daltons. In certain embodiments, an antigen-binding domain comprises molecular weight of less than about 16,000 Daltons. In certain embodiments, an antigen-binding domain comprises molecular weight of less than about 15,000 Daltons.

In certain embodiments, an antigen-binding domain comprises molecular weight of about 12,000 Daltons to about 25,000 Daltons. In certain embodiments, an antigen-binding domain comprises molecular weight of about 12,000 Daltons to about 13,000 Daltons, about 12,000 Daltons to about 14,000 Daltons, about 12,000 Daltons to about 15,000 Daltons, about 12,000 Daltons to about 16,000 Daltons, about 12,000 Daltons to about 17,000 Daltons, about 12,000 Daltons to about 18,000 Daltons, about 12,000 Daltons to about 19,000 Daltons, about 12,000 Daltons to about 20,000 Daltons, about 12,000 Daltons to about 25,000 Daltons, about 13,000 Daltons to about 14,000 Daltons, about 13,000 Daltons to about 15,000 Daltons, about 13,000 Daltons to about 16,000 Daltons, about 13,000 Daltons to about 17,000 Daltons, about 13,000 Daltons to about 18,000 Daltons, about 13,000 Daltons to about 19,000 Daltons, about 13,000 Daltons to about 20,000 Daltons, about 13,000 Daltons to about 25,000 Daltons, about 14,000 Daltons to about 15,000 Daltons, about 14,000 Daltons to about 16,000 Daltons, about 14,000 Daltons to about 17,000 Daltons, about 14,000 Daltons to about 18,000 Daltons, about 14,000 Daltons to about 19,000 Daltons, about 14,000 Daltons to about 20,000 Daltons, about 14,000 Daltons to about 25,000 Daltons, about 15,000 Daltons to about 16,000 Daltons, about 15,000 Daltons to about 17,000 Daltons, about 15,000 Daltons to about 18,000 Daltons, about 15,000 Daltons to about 19,000 Daltons, about 15,000 Daltons to about 20,000 Daltons, about 15,000 Daltons to about 25,000 Daltons, about 16,000 Daltons to about 17,000 Daltons, about 16,000 Daltons to about 18,000 Daltons, about 16,000 Daltons to about 19,000 Daltons, about 16,000 Daltons to about 20,000 Daltons, about 16,000 Daltons to about 25,000 Daltons, about 17,000 Daltons to about 18,000 Daltons, about 17,000 Daltons to about 19,000 Daltons, about 17,000 Daltons to about 20,000 Daltons, about 17,000 Daltons to about 25,000 Daltons, about 18,000 Daltons to about 19,000 Daltons, about 18,000 Daltons to about 20,000 Daltons, about 18,000 Daltons to about 25,000 Daltons, about 19,000 Daltons to about 20,000 Daltons, about 19,000 Daltons to about 25,000 Daltons, or about 20,000 Daltons to about 25,000 Daltons. In certain embodiments, an antigen-binding domain comprises molecular weight of about 12,000 Daltons, about 13,000 Daltons, about 14,000 Daltons, about 15,000 Daltons, about 16,000 Daltons, about 17,000 Daltons, about 18,000 Daltons, about 19,000 Daltons, about 20,000 Daltons, or about 25,000 Daltons.

In certain embodiments, the antigen-binding domain comprises a single-chain variable domain polypeptide selected from the group consisting of a scFv, VH, VL, VHH, and VNAR. In certain embodiments, the antigen-binding domain comprises an immunoglobulin single-chain variable domain polypeptide. In certain embodiments, the immunoglobulin single-chain variable domain polypeptide is selected from the group consisting of a VH, VL, and VHH. In certain embodiments, In certain embodiments, the antigen-binding domain comprises a VHH (e.g., a first VHH domain and a second VHH domain).

For example, in certain embodiments, the first antigen-binding domain and second antigen-binding domain each comprise a single-chain variable domain polypeptide selected from the group consisting of a scFv, VH, VL, VHH, and VNAR. In certain embodiments, the first antigen-binding domain and second antigen-binding domain each comprise an immunoglobulin single-chain variable domain polypeptide. In certain embodiments, the immunoglobulin single-chain variable domain polypeptide is selected from the group consisting of a VH, VL, and VHH. In certain embodiments, the first antigen-binding domain and second antigen-binding domain each comprise a VHH (e.g., a first VHH domain and a second VHH domain).

Provided herein are antigen-binding domains that bind FOLR1. “FOLR1” or “Folate receptor alpha” or MOv18” or Folate Receptor 1” refers to and encompasses the protein encoded by the FOLR1 gene (see NC_000011.10 (72189709 . . . 72196323); NCBI Gene 2348, or UniProt ID P15328).

In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the Kabat definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the Chothia definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the AbM definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the Contact definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the IMGT definition.

In some embodiments, the antigen-binding domain is an immunoglobulin single-chain domain comprising: a complementarity determining region (CDR) 1 comprising the amino acid sequence as set forth in SEQ ID NO: 1; a complementarity determining region (CDR) 2 comprising the amino acid sequence as set forth in SEQ ID NO: 2; and a complementarity determining region (CDR) 3 comprising the amino acid sequence as set forth in SEQ ID NO: 3, wherein the immunoglobulin single-chain domain binds FOLR1.

In certain embodiments, the antigen-binding domain is an immunoglobulin single-chain domain comprising an amino acid sequence having at least 70% sequence identity to SEQ ID NO: 4. In certain embodiments, the antigen-binding domain is an immunoglobulin single-chain domain comprising an amino acid sequence having at least 80% sequence identity to SEQ ID NO: 4. In certain embodiments, the antigen-binding domain is an immunoglobulin single-chain domain comprising an amino acid sequence having at least 85% sequence identity to SEQ ID NO: 4. In certain embodiments, the antigen-binding domain is an immunoglobulin single-chain domain comprising an amino acid sequence having at least 90% sequence identity to SEQ ID NO: 4. In certain embodiments, the antigen-binding domain is an immunoglobulin single-chain domain comprising an amino acid sequence having at least 95% sequence identity to SEQ ID NO: 4. In certain embodiments, the antigen-binding domain is an immunoglobulin single-chain domain comprising an amino acid sequence having at least 97% sequence identity to SEQ ID NO: 4. In certain embodiments, the antigen-binding domain is an immunoglobulin single-chain domain comprising an amino acid sequence having at least 98% sequence identity to SEQ ID NO: 4. In certain embodiments, the antigen-binding domain is an immunoglobulin single-chain domain comprising an amino acid sequence having at least 99% sequence identity to SEQ ID NO: 4. In certain embodiments, the antigen-binding domain is an immunoglobulin single-chain domain comprising an amino acid sequence as set forth in SEQ ID NO: 4.

Provided herein are antigen-binding domains that bind HER2. “HER2” or “ERBB2” or “erb-b2 receptor tyrosine kinase 2” refers to and encompasses the protein encoded by the HER2 gene (see NC_000017.11 (39688094..39728658); NCBI Gene 2064, or UniProt ID Q9UK79).

In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 24, wherein the CDR1-3 are defined using the Kabat definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 24, wherein the CDR1-3 are defined using the Chothia definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 24, wherein the CDR1-3 are defined using the AbM definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 24, wherein the CDR1-3 are defined using the Contact definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 24, wherein the CDR1-3 are defined using the IMGT definition.

In some embodiments, the antigen-binding domain is an immunoglobulin single-chain domain comprising: a complementarity determining region (CDR) 1 comprising the amino acid sequence as set forth in SEQ ID NO: 21; a complementarity determining region (CDR) 2 comprising the amino acid sequence as set forth in SEQ ID NO: 22; and a complementarity determining region (CDR) 3 comprising the amino acid sequence as set forth in SEQ ID NO: 23, wherein the immunoglobulin single-chain domain binds HER2.

In certain embodiments, the antigen-binding domain is an immunoglobulin single-chain domain comprising an amino acid sequence having at least 70% sequence identity to SEQ ID NO: 24. In certain embodiments, the antigen-binding domain is an immunoglobulin single-chain domain comprising an amino acid sequence having at least 80% sequence identity to SEQ ID NO: 24. In certain embodiments, the antigen-binding domain is an immunoglobulin single-chain domain comprising an amino acid sequence having at least 85% sequence identity to SEQ ID NO: 24. In certain embodiments, the antigen-binding domain is an immunoglobulin single-chain domain comprising an amino acid sequence having at least 90% sequence identity to SEQ ID NO: 24. In certain embodiments, the antigen-binding domain is an immunoglobulin single-chain domain comprising an amino acid sequence having at least 95% sequence identity to SEQ ID NO: 24. In certain embodiments, the antigen-binding domain is an immunoglobulin single-chain domain comprising an amino acid sequence having at least 97% sequence identity to SEQ ID NO: 24. In certain embodiments, the antigen-binding domain is an immunoglobulin single-chain domain comprising an amino acid sequence having at least 98% sequence identity to SEQ ID NO: 24. In certain embodiments, the antigen-binding domain is an immunoglobulin single-chain domain comprising an amino acid sequence having at least 99% sequence identity to SEQ ID NO: 24. In certain embodiments, the antigen-binding domain is an immunoglobulin single-chain domain comprising an amino acid sequence as set forth in SEQ ID NO: 24.

Provided herein are antigen-binding domains that bind DLL3. “DLL3” or “delta like canonical Notch ligand 3” or SCDO1” refers to and encompasses the protein encoded by the DLL3 gene (see NC_000019.10 (39498947..39508469); NCBI Gene 10683, or UniProt ID Q9NYJ7).

In some embodiments, the first antigen-binding domain or the second antigen-binding domain comprise an amino acid sequence at least about 85%, 90%, 95%, 97%, 98%, 99%, or 100% identical to that set forth in SEQ ID NO: 4. In some embodiments, the first antigen-binding domain or the second antigen-binding domain comprise an amino acid sequence at least about 85%, 90%, 95%, 97%, 98%, 99%, or 100% identical to that set forth in SEQ ID NO: 8, 101-106, 201-206, 301-306, 401-406, or 501-506. In some embodiments, the first antigen-binding domain comprises an amino acid sequence at least about 85%, 90%, 95%, 97%, 98%, 99%, or 100% identical to that set forth in SEQ ID NO: 4; and the second antigen-binding domain comprises an amino acid sequence at least about 85%, 90%, 95%, 97%, 98%, 99%, or 100% identical to that set forth in SEQ ID NO: 8, 101-106, 201-206, 301-306, 401-406, or 501-506.

In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 8, 101-106, 201-206, 301-306, 401-406, or 501-506, wherein the CDR1-3 are defined using the Kabat definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 8, 101-106, 201-206, 301-306, 401-406, or 501-506, wherein the CDR1-3 are defined using the Chothia definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 8, 101-106, 201-206, 301-306, 401-406, or 501-506, wherein the CDR1-3 are defined using the AbM definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 8, 101-106, 201-206, 301-306, 401-406, or 501-506, wherein the CDR1-3 are defined using the Contact definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 8, 101-106, 201-206, 301-306, 401-406, or 501-506, wherein the CDR1-3 are defined using the IMGT definition.

In some embodiments, an antigen-binding domain comprises: a complementarity determining region (CDR) 1 comprising the amino acid sequence as set forth in SEQ ID NO: 5; a complementarity determining region (CDR) 2 comprising the amino acid sequence as set forth in SEQ ID NO: 6; and a complementarity determining region (CDR) 3 comprising the amino acid sequence as set forth in SEQ ID NO: 7; a complementarity determining region (CDR) 1 comprising the amino acid sequence as set forth in SEQ ID NO: 107; a complementarity determining region (CDR) 2 comprising the amino acid sequence as set forth in SEQ ID NO: 110; and a complementarity determining region (CDR) 3 comprising the amino acid sequence as set forth in SEQ ID NO: 113; a complementarity determining region (CDR) 1 comprising the amino acid sequence as set forth in SEQ ID NO: 207; a complementarity determining region (CDR) 2 comprising the amino acid sequence as set forth in SEQ ID NO: 210; and a complementarity determining region (CDR) 3 comprising the amino acid sequence as set forth in SEQ ID NO: 213; a complementarity determining region (CDR) 1 comprising the amino acid sequence as set forth in SEQ ID NO: 307; a complementarity determining region (CDR) 2 comprising the amino acid sequence as set forth in SEQ ID NO: 310; and a complementarity determining region (CDR) 3 comprising the amino acid sequences as set forth in SEQ ID NO: 313; a complementarity determining region (CDR) 1 comprising the amino acid sequence as set forth in SEQ ID NO: 407; a complementarity determining region (CDR) 2 comprising the amino acid sequence as set forth in SEQ ID NO: 410; and a complementarity determining region (CDR) 3 comprising the amino acid sequence as set forth in SEQ ID NO: 413; or a complementarity determining region (CDR) 1 comprising the amino acid sequence as set forth in SEQ ID NO: 507; a complementarity determining region (CDR) 2 comprising the amino acid sequence as set forth in SEQ ID NO: 510; and a complementarity determining region (CDR) 3 comprising the amino acid sequence as set forth in SEQ ID NO: 513.

In some embodiments, the multivalent peptide is monospecific, comprising a first antigen-binding domain and a second antigen-binding domain that bind to the same target (e.g., antigen). In some embodiments, the multivalent peptide is monospecific, comprising a first antigen-binding domain and a second antigen-binding domain that bind to FOLR1. In some embodiments, the multivalent peptide is monospecific, comprising a first antigen-binding domain and a second antigen-binding domain that bind to DLL3. In some embodiments, the multivalent peptide is monospecific, comprising a first antigen-binding domain and a second antigen-binding domain that bind to HER2.

In some embodiments, the multivalent peptide is bispecific, comprising a first antigen-binding domain and a second antigen-binding domain that bind to a different target (e.g., antigen). In some embodiments, the multivalent peptide is bispecific, comprising a first antigen-binding domain that binds FOLR1 and a second antigen-binding domain that bind to DLL3.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 407, (2) a CDR2 comprising SEQ ID NO: 410, and (3) a CDR3 comprising SEQ ID NO: 413. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 408, (2) a CDR2 comprising SEQ ID NO: 411, and (3) a CDR3 comprising SEQ ID NO: 414. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 409, (2) a CDR2 comprising SEQ ID NO: 412, and (3) a CDR3 comprising SEQ ID NO: 415.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 307, (2) a CDR2 comprising SEQ ID NO: 310, and (3) a CDR3 comprising SEQ ID NO: 313. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 308, (2) a CDR2 comprising SEQ ID NO: 311, and (3) a CDR3 comprising SEQ ID NO: 314. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 309, (2) a CDR2 comprising SEQ ID NO: 312, and (3) a CDR3 comprising SEQ ID NO: 315.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 207, (2) a CDR2 comprising SEQ ID NO: 210, and (3) a CDR3 comprising SEQ ID NO: 213. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 208, (2) a CDR2 comprising SEQ ID NO: 211, and (3) a CDR3 comprising SEQ ID NO: 214. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 209, (2) a CDR2 comprising SEQ ID NO: 212, and (3) a CDR3 comprising SEQ ID NO: 215.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 507, (2) a CDR2 comprising SEQ ID NO: 510, and (3) a CDR3 comprising SEQ ID NO: 513. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 508, (2) a CDR2 comprising SEQ ID NO: 511, and (3) a CDR3 comprising SEQ ID NO: 514. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 509, (2) a CDR2 comprising SEQ ID NO: 512, and (3) a CDR3 comprising SEQ ID NO: 515.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 107, (2) a CDR2 comprising SEQ ID NO: 110, and (3) a CDR3 comprising SEQ ID NO: 113. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 108, (2) a CDR2 comprising SEQ ID NO: 111, and (3) a CDR3 comprising SEQ ID NO: 114. In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 109, (2) a CDR2 comprising SEQ ID NO: 112, and (3) a CDR3 comprising SEQ ID NO: 115.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds DLL3 comprises: (1) a CDR1 comprising SEQ ID NO: 5, (2) a CDR2 comprising SEQ ID NO: 6, and (3) a CDR3 comprising SEQ ID NO: 7.

In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 403. In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 303. In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 304. In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 305. In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 503. In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 103. In certain embodiments, the antigen-binding domain that binds FOLR1 comprises a VHH comprising a sequence having at least 90% sequence identity to SEQ ID NO: 4; and antigen-binding domain that binds DLL3 comprises a VHH comprising a sequence having at least 90% sequence identity to SEQ ID NO: 8.

In certain embodiments, the antigen-binding domain that binds FOLR1 comprises a VHH comprising SEQ ID NO: 4; and antigen-binding domain that binds DLL3 comprises a VHH comprising SEQ ID NO: 403. In certain embodiments, the antigen-binding domain that binds FOLR1 comprises a VHH comprising SEQ ID NO: 4; and antigen-binding domain that binds DLL3 comprises a VHH comprising SEQ ID NO: 303. In certain embodiments, the antigen-binding domain that binds FOLR1 comprises a VHH comprising SEQ ID NO: 4; and antigen-binding domain that binds DLL3 comprises a VHH comprising SEQ ID NO: 304. In certain embodiments, the antigen-binding domain that binds FOLR1 comprises a VHH comprising SEQ ID NO: 4; and antigen-binding domain that binds DLL3 comprises a VHH comprising SEQ ID NO: 305. In certain embodiments, the antigen-binding domain that binds FOLR1 comprises a VHH comprising SEQ ID NO: 4; and antigen-binding domain that binds DLL3 comprises a VHH comprising SEQ ID NO: 503. In certain embodiments, the antigen-binding domain that binds FOLR1 comprises a VHH comprising SEQ ID NO: 4; and antigen-binding domain that binds DLL3 comprises a VHH comprising SEQ ID NO: 103. In certain embodiments, the antigen-binding domain that binds FOLR1 comprises a VHH comprising SEQ ID NO: 4; and antigen-binding domain that binds DLL3 comprises a VHH comprising SEQ ID NO: 8.

In some embodiments, the multivalent peptide is bispecific, comprising a first antigen-binding domain and a second antigen-binding domain that bind to a different target (e.g., antigen). In some embodiments, the multivalent peptide is bispecific, comprising a first antigen-binding domain that binds FOLR1 and a second antigen-binding domain that bind to HER2.

In certain embodiments, the antigen-binding domain that binds FOLR1 comprises: (1) a CDR1 comprising SEQ ID NO: 1, (2) a CDR2 comprising SEQ ID NO: 2, and (3) a CDR3 comprising SEQ ID NO: 3; and the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23. In certain embodiments, the antigen-binding domain that binds FOLR1 comprises a VHH comprising a sequence having at least 90% sequence identity to SEQ ID NO: 4; and antigen-binding domain that binds HER2 comprises a VHH comprising a sequence having at least 90% sequence identity to SEQ ID NO: 24. In certain embodiments, the antigen-binding domain that binds FOLR1 comprises a VHH comprising SEQ ID NO: 4; and antigen-binding domain that binds HER2 comprises a VHH comprising SEQ ID NO: 24.

In some embodiments, the multivalent peptide is bispecific, comprising a first antigen-binding domain and a second antigen-binding domain that bind to a different target (e.g., antigen). In some embodiments, the multivalent peptide is bispecific, comprising a first antigen-binding domain that binds HER2 and a second antigen-binding domain that bind to DLL3.

In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 407, (2) a CDR2 comprising SEQ ID NO: 410, and (3) a CDR3 comprising SEQ ID NO: 413. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 408, (2) a CDR2 comprising SEQ ID NO: 411, and (3) a CDR3 comprising SEQ ID NO: 414. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 409, (2) a CDR2 comprising SEQ ID NO: 412, and (3) a CDR3 comprising SEQ ID NO: 415.

In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 307, (2) a CDR2 comprising SEQ ID NO: 310, and (3) a CDR3 comprising SEQ ID NO: 313. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 308, (2) a CDR2 comprising SEQ ID NO: 311, and (3) a CDR3 comprising SEQ ID NO: 314. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 309, (2) a CDR2 comprising SEQ ID NO: 312, and (3) a CDR3 comprising SEQ ID NO: 315.

In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 207, (2) a CDR2 comprising SEQ ID NO: 210, and (3) a CDR3 comprising SEQ ID NO: 213. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 208, (2) a CDR2 comprising SEQ ID NO: 211, and (3) a CDR3 comprising SEQ ID NO: 214. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 209, (2) a CDR2 comprising SEQ ID NO: 212, and (3) a CDR3 comprising SEQ ID NO: 215.

In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 507, (2) a CDR2 comprising SEQ ID NO: 510, and (3) a CDR3 comprising SEQ ID NO: 513. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 508, (2) a CDR2 comprising SEQ ID NO: 511, and (3) a CDR3 comprising SEQ ID NO: 514. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 509, (2) a CDR2 comprising SEQ ID NO: 512, and (3) a CDR3 comprising SEQ ID NO: 515.

In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 107, (2) a CDR2 comprising SEQ ID NO: 110, and (3) a CDR3 comprising SEQ ID NO: 113. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 108, (2) a CDR2 comprising SEQ ID NO: 111, and (3) a CDR3 comprising SEQ ID NO: 114. In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 109, (2) a CDR2 comprising SEQ ID NO: 112, and (3) a CDR3 comprising SEQ ID NO: 115.

In certain embodiments, the antigen-binding domain that binds HER2 comprises: (1) a CDR1 comprising SEQ ID NO: 21, (2) a CDR2 comprising SEQ ID NO: 22, and (3) a CDR3 comprising SEQ ID NO: 23; and the antigen-binding domain that binds DLL3 comprises: (1) a CDR1 comprising SEQ ID NO: 5, (2) a CDR2 comprising SEQ ID NO: 6, and (3) a CDR3 comprising SEQ ID NO: 7.

In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 403. In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 303. In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 304. In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 305. In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 503. In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 103. In certain embodiments, the antigen-binding domain that binds HER2 comprises a VHH comprising a sequence having at least 90% sequence identity to SEQ ID NO: 24; and antigen-binding domain that binds DLL3 comprises a VHH comprising a sequence having at least 90% sequence identity to SEQ ID NO: 8.

In certain embodiments, the antigen-binding domain that binds HER2 comprises a VHH comprising SEQ ID NO: 24; and antigen-binding domain that binds DLL3 comprises a VHH comprising SEQ ID NO: 403. In certain embodiments, the antigen-binding domain that binds HER2 comprises a VHH comprising SEQ ID NO: 24; and antigen-binding domain that binds DLL3 comprises a VHH comprising SEQ ID NO: 303. In certain embodiments, the antigen-binding domain that binds HER2 comprises a VHH comprising SEQ ID NO: 24; and antigen-binding domain that binds DLL3 comprises a VHH comprising SEQ ID NO: 304. In certain embodiments, the antigen-binding domain that binds HER2 comprises a VHH comprising SEQ ID NO: 24; and antigen-binding domain that binds DLL3 comprises a VHH comprising SEQ ID NO: 305. In certain embodiments, the antigen-binding domain that binds HER2 comprises a VHH comprising SEQ ID NO: 24; and antigen-binding domain that binds DLL3 comprises a VHH comprising SEQ ID NO: 503. In certain embodiments, the antigen-binding domain that binds HER2 comprises a VHH comprising SEQ ID NO: 24; and antigen-binding domain that binds DLL3 comprises a VHH comprising SEQ ID NO: 103. In certain embodiments, the antigen-binding domain that binds HER2 comprises a VHH comprising SEQ ID NO: 24; and antigen-binding domain that binds DLL3 comprises a VHH comprising SEQ ID NO: 8.

### Fc Domains

A “Fc region” (fragment crystallizable region) or “Fc domain” or “Fc” refers to and encompasses the C-terminal, non-antigen-binding region of the heavy chain of an antibody that mediates the binding of the immunoglobulin to host tissues or factors, including binding to Fc receptors located on various cells of the immune system (e.g., effector cells) or to the first component (Clq) of the classical complement system. An antibody constant region generally comprises the CL region (e.g., for light chains) or CH1-CH2-CH3 regions. Generally, an Fc domain generally refers to and encompasses the CH2-CH3 regions of a heavy chain constant region. For IgG, the Fc domain comprises immunoglobulin domains CH2 and CH3 (Cy2 and Cy3), and optionally all or a portion of the hinge region between CHI (Cy1) and CH2 (Cy2). In some embodiments, the Fc domain includes, from N- to C-terminus, CH2-CH3 and hinge-CH2-CH3. In some embodiments, the Fc domain is derived from IgG1, IgG2, IgG3 or IgG4, comprising the hinge-CH2-CH3 domains/regions. Additionally, in certain embodiments, wherein the Fc domain is a human IgG1 Fc domain, the hinge includes a C220S ammo acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU. In some embodiments, as is more fully described below, amino acid modifications are made to the Fc region, for example to alter binding to one or more FcyR or to the FcRn. An Fc domain can be a native sequence Fc, including any allotypic variant, or a variant Fc (comprising one or more mutations that reduce effector cell function and/or FcRN).

A “hinge” or “hinge region” or “antibody hinge region” or “immunoglobulin hinge region” refers to and encompasses the flexible polypeptide comprising the amino acids between the first (CH1) and second (CH2) heavy chain constant domains of an antibody. Structurally, the IgG CH1 domain ends at EU position 215, and the IgG CH2 domain begins at residue EU position 231. For IgG, the antibody hinge includes positions 216 (E216 in IgGl) to 230 (P230 in IgGl), wherein the numbering is according to the EU index as in Kabat. In some embodiments, for example in the context of an Fc region, the hinge (full length or a fragment of the hinge) is included, generally referring to positions 216-230.

An “isotype” refers to and encompasses the antibody class (e.g., IgGl, IgG2, IgG3, IgG4, IgM, IgA1, IgA2, IgD, and IgE antibody) that is encoded by the heavy chain constant domain genes. The full-length amino acid sequence of each wild-type human IgG constant region (including all domains, i.e., CHI domain, hinge, CH2 domain, and CH3 domain) is cataloged in the UniProt database available on-line, e.g., as P01857 (IgGl), P01859 (IgG2), P01860 (IgG3), and P01861 (IgG4), or different allotypes thereof. A domain of a heavy chain constant region is of an “IgG1 isotype,” “IgG2 isotype,” “IgG3 isotype,” or “IgG4 isotype,” if the domain comprises the amino acid sequence of the corresponding domain of the respective isotype, or a variant thereof (that has a higher homology to the corresponding domain of the respective isotype than it does to that of the other isotypes). An “allotype” refers to naturally occurring variants within a specific isotype group, which variants differ in a few amino acids (see, e.g., Jefferies et al. (2009) mAbs 1:1). In certain embodiments, the immunoglobulin heavy chain constant region is a human immunoglobulin heavy chain constant region. In certain embodiments, the immunoglobulin heavy chain constant region is an IgA, IgG1, IgG2, IgG3, or IgG4 isotype. In certain embodiments, the immunoglobulin heavy chain constant region is an IgG1 isotype. In certain embodiments, the immunoglobulin heavy chain constant region is an IgG4 isotype.

In some embodiments, the Fc domain comprises a variant Fc domain comprising one or mutations that modulate (e.g., reduce, inhibit, decrease, prevent, etc.) effector function associated with a heavy chain constant region, FcRn binding, or both.

The immunoglobulin heavy chain constant region can be a variant constant region that comprises one or more alterations to an amino acid residues that confers additional utility and advantageous properties to the immunoconjugates described herein. In certain embodiments, the immunoglobulin heavy chain constant region comprises an alteration to one or more amino acid residues that reduces an effector function of the immunoglobulin heavy chain constant region or alters binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region or reduces binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region and reduces binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region. In certain embodiments, the immunoglobulin heavy chain constant region comprises an alteration to one or more amino acid residues that reduces binding of the immunoconjugate to the neonatal Fc receptor (FcRn).

In some embodiments, the Fc domain comprises alterations to a heavy chain constant region that reduces effector function associated with a heavy chain constant region, such as, the ability to fix complement, promote phagocytosis, or recruit other immune effector cells (e.g., NK cells) to the heavy chain constant region. In certain embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof. In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (j) 373A, 373G, or 373S, (kk) 376E, 376W, or 376Y, (11) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ooo), per EU numbering. In certain embodiments, the alteration to one or more amino acid residues that reduces the effector function of the immunoglobulin heavy chain constant region comprises L234A, L235E, G237A, A330S, and P331S per EU numbering.

In some embodiments, the Fc domain comprises alterations to a heavy chain constant region that reduces the serum half-life of the immunoconjugate. In certain embodiments, the amino acid alteration that alters or reduces binding of the immunoconjugate to the neonatal Fc receptor (FcRn) reduces the serum half-life of the immunoconjugate. In certain embodiments, the alteration that alters or reduces binding of the immunoconjugate to the neonatal Fc receptor (FcRn) is to an amino acid residue selected from the list consisting of. 251, 252, 253, 254, 255, 288, 309, 310, 312, 385, 386, 388, 400, 415, 433, 435, 436, 439, 447, and combinations thereof per EU numbering. In certain embodiments, the alteration that alters or reduces binding of the immunoconjugate to the neonatal Fc receptor (FcRn) is to an amino acid residue selected from the list consisting of. 253, 254, 310, 435, 436 and combinations thereof per EU numbering. In certain embodiments, the alteration that alters or reduces binding of the immunoconjugate to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration that alters or reduces binding of the immunoconjugate to the neonatal Fc receptor (FcRn) is to an amino acid residue selected from the list consisting of: I253A, S254A, H310A, H435Q, Y436A and combinations thereof per EU numbering. In certain embodiments, the alteration that alters or reduces binding of the immunoconjugate to the neonatal Fc receptor (FcRn) is to an amino acid residue selected from the list consisting of: I253A, H310A, H435Q, and combinations thereof per EU numbering. In certain embodiments, the alteration that alters or reduces binding of the immunoconjugate to the neonatal Fc receptor (FcRn) is to an amino acid residue selected from the list consisting of: H310A, H435Q, and combinations thereof per EU numbering.

In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 2355, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 31 IS, (ee) 316F, (ff) 328V, (gg) 330R, (hh) 339E or 339L, (ii) 343I or 343V, (jj) 373A, 373G, or 3735, (kk) 376E, 376W, or 376Y, (11) 380D, (mm) 382D or 382P, (nn) 385P, (oo) 424H, 424M, or 424V, (pp) 4341, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ppp), per EU numbering.

In certain embodiments, the alteration to one or more amino acid residues that reduces the effector function of the immunoglobulin heavy chain constant region comprises L234A, L235E, G237A, A330S, and P331S per EU numbering. In certain embodiments, the Fc domain comprises an alteration to one or more amino acid residues that alters binding of the immunoconjugate to the neonatal Fc receptor (FcRn). In certain embodiments, the alteration to one or more amino acid residues that alters binding of the immunoconjugate to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, S254A, H310A, H435Q, Y436A and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, H310A, H435Q, and combinations thereof per EU numbering. In certain embodiments, the alteration to one or more amino acid residues that alters binding of the immunoconjugate to the neonatal Fc receptor (FcRn) comprises I253A per EU numbering. In certain embodiments, the alteration to one or more amino acid residues that alters binding of the immunoconjugate to the neonatal Fc receptor (FcRn) comprises H310A per EU numbering. In certain embodiments, the alteration to one or more amino acid residues that alters binding of the immunoconjugate to the neonatal Fc receptor (FcRn) comprises H435Q per EU numbering.

In certain embodiments, a heavy chain constant regions of the immunoconjugate comprises a sequence at least 90%, 95%, 97%, 98%, or 99% identical to the sequence set forth in SEQ ID NO: 11. In certain embodiments, a heavy chain constant regions of the immunoconjugate comprises a sequence identical to SEQ ID NO: 11. In certain embodiments, a heavy chain constant regions of the immunoconjugate comprises a sequence at least 90%, 95%, 97%, 98%, or 99% identical to the sequence set forth in SEQ ID NO: 11, wherein the heavy chain constant region comprises an I253A substitution per EU numbering.

In certain embodiments, a heavy chain constant regions of the immunoconjugate comprises a sequence at least 90%, 95%, 97%, 98%, or 99% identical to the sequence set forth in SEQ ID NO: 12. In certain embodiments, a heavy chain constant regions of the immunoconjugate comprises a sequence identical to SEQ ID NO: 12. In certain embodiments, a heavy chain constant regions of the immunoconjugate comprises a sequence at least 90%, 95%, 97%, 98%, or 99% identical to the sequence set forth in SEQ ID NO: 12, wherein the heavy chain constant region comprises an S254A substitution per EU numbering.

In certain embodiments, a heavy chain constant regions of the immunoconjugate comprises a sequence at least 90%, 95%, 97%, 98%, or 99% identical to the sequence set forth in SEQ ID NO: 13. In certain embodiments, a heavy chain constant regions of the immunoconjugate comprises a sequence identical to SEQ ID NO: 13. In certain embodiments, a heavy chain constant regions of the immunoconjugate comprises a sequence at least 90%, 95%, 97%, 98%, or 99% identical to the sequence set forth in SEQ ID NO: 13, wherein the heavy chain constant region comprises an H310A substitution per EU numbering.

In certain embodiments, a heavy chain constant regions of the immunoconjugate comprises a sequence at least 90%, 95%, 97%, 98%, or 99% identical to the sequence set forth in SEQ ID NO: 14. In certain embodiments, a heavy chain constant regions of the immunoconjugate comprises a sequence identical to SEQ ID NO: 14. In certain embodiments, a heavy chain constant regions of the immunoconjugate comprises a sequence at least 90%, 95%, 97%, 98%, or 99% identical to the sequence set forth in SEQ ID NO: 14, wherein the heavy chain constant region comprises an H435Q substitution per EU numbering.

In certain embodiments, a heavy chain constant regions of the immunoconjugate comprises a sequence at least 90%, 95%, 97%, 98%, or 99% identical to the sequence set forth in SEQ ID NO: 15. In certain embodiments, a heavy chain constant regions of the immunoconjugate comprises a sequence identical to SEQ ID NO: 15. In certain embodiments, a heavy chain constant regions of the immunoconjugate comprises a sequence at least 90%, 95%, 97%, 98%, or 99% identical to the sequence set forth in SEQ ID NO: 15, wherein the heavy chain constant region comprises an Y436A substitution per EU numbering.

In certain embodiments, a heavy chain constant regions of the immunoconjugate comprises a sequence at least 90%, 95%, 97%, 98%, or 99% identical to the sequence set forth in SEQ ID NO: 16. In certain embodiments, a heavy chain constant regions of the immunoconjugate comprises a sequence identical to SEQ ID NO: 16. In certain embodiments, a heavy chain constant regions of the immunoconjugate comprises a sequence at least 90%, 95%, 97%, 98%, or 99% identical to the sequence set forth in SEQ ID NO: 16, wherein the heavy chain constant region comprises an H310A/H435Q substitution per EU numbering.

In certain embodiments, a heavy chain constant regions of the immunoconjugate comprises a sequence at least 90%, 95%, 97%, 98%, or 99% identical to the sequence set forth in SEQ ID NO: 17. In certain embodiments, a heavy chain constant regions of the immunoconjugate comprises a sequence identical to SEQ ID NO: 17. In certain embodiments, a heavy chain constant regions of the immunoconjugate comprises a sequence at least 90%, 95%, 97%, 98%, or 99% identical to the sequence set forth in SEQ ID NO: 17, wherein the heavy chain constant region comprises a L234A, L235E, G237A, A330S, and P331S substitution per EU numbering.

In certain embodiments, a heavy chain constant regions of the immunoconjugate comprises a sequence at least 90%, 95%, 97%, 98%, or 99% identical to the sequence set forth in SEQ ID NO: 18. In certain embodiments, a heavy chain constant regions of the immunoconjugate comprises a sequence identical to SEQ ID NO: 18, wherein the heavy chain constant region comprises a L234A, L235E, G237A, H310A, A330S, and P331S substitution per EU numbering.

In certain embodiments, a heavy chain constant regions of the immunoconjugate comprises a sequence at least 90%, 95%, 97%, 98%, or 99% identical to the sequence set forth in SEQ ID NO: 19. In certain embodiments, a heavy chain constant regions of the immunoconjugate comprises a sequence identical to SEQ ID NO: 19. In certain embodiments, a heavy chain constant regions of the immunoconjugate comprises a sequence identical to SEQ ID NO: 19, wherein the heavy chain constant region comprises a L234A, L235E, G237A, H435Q, A330S, and P331S substitution per EU numbering.

In certain embodiments, a heavy chain constant regions of the immunoconjugate comprises a sequence at least 90%, 95%, 97%, 98%, or 99% identical to the sequence set forth in SEQ ID NO: 20. In certain embodiments, a heavy chain constant regions of the immunoconjugate comprises a sequence identical to SEQ ID NO: 20 per EU numbering.

Alterations that effect FcRn binding can reduce the serum half-life of the immunoconjugate, thus allowing the skilled artisan to choose a half-life that is suitable for a particular imaging or therapeutic goal. In certain embodiments, the immunoconjugate has a serum half-life of about 12 hours to about 120 hours. In certain embodiments, the immunoconjugate has a serum half-life of about 12 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 12 hours to about 108 hours, about 12 hours to about 120 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 24 hours to about 108 hours, about 24 hours to about 120 hours, about 36 hours to about 48 hours, about 36 hours to about 60 hours, about 36 hours to about 72 hours, about 36 hours to about 84 hours, about 36 hours to about 96 hours, about 36 hours to about 108 hours, about 36 hours to about 120 hours, about 48 hours to about 60 hours, about 48 hours to about 72 hours, about 48 hours to about 84 hours, about 48 hours to about 96 hours, about 48 hours to about 108 hours, about 48 hours to about 120 hours, about 60 hours to about 72 hours, about 60 hours to about 84 hours, about 60 hours to about 96 hours, about 60 hours to about 108 hours, about 60 hours to about 120 hours, about 72 hours to about 84 hours, about 72 hours to about 96 hours, about 72 hours to about 108 hours, about 72 hours to about 120 hours, about 84 hours to about 96 hours, about 84 hours to about 108 hours, about 84 hours to about 120 hours, about 96 hours to about 108 hours, about 96 hours to about 120 hours, or about 108 hours to about 120 hours. In certain embodiments, the immunoconjugate has a serum half-life of about 12 hours, about 24 hours, about 36 hours, about 48 hours, about 60 hours, about 72 hours, about 84 hours, about 96 hours, about 108 hours, or about 120 hours. In certain embodiments, the immunoconjugate has a serum half-life of at least about 12 hours, about 24 hours, about 36 hours, about 48 hours, about 60 hours, about 72 hours, about 84 hours, about 96 hours, or about 108 hours. In certain embodiments, the immunoconjugate has a serum half-life of at most about 24 hours, about 36 hours, about 48 hours, about 60 hours, about 72 hours, about 84 hours, about 96 hours, about 108 hours, or about 120 hours.

In certain embodiments, the immunoconjugate has a serum half-life of about 1 day to about 10 days. In certain embodiments, the immunoconjugate has a serum half-life of about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 3 days to about 4 days, about 3 days to about 5 days, about 3 days to about 6 days, about 3 days to about 7 days, about 3 days to about 8 days, about 3 days to about 9 days, about 3 days to about 10 days, about 4 days to about 5 days, about 4 days to about 6 days, about 4 days to about 7 days, about 4 days to about 8 days, about 4 days to about 9 days, about 4 days to about 10 days, about 5 days to about 6 days, about 5 days to about 7 days, about 5 days to about 8 days, about 5 days to about 9 days, about 5 days to about 10 days, about 6 days to about 7 days, about 6 days to about 8 days, about 6 days to about 9 days, about 6 days to about 10 days, about 7 days to about 8 days, about 7 days to about 9 days, about 7 days to about 10 days, about 8 days to about 9 days, about 8 days to about 10 days, or about 9 days to about 10 days. In certain embodiments, the immunoconjugate has a serum half-life of about 1 day, about 2 days, about 3 days, about 4 days, about 5 days, about 6 days, about 7 days, about 8 days, about 9 days, or about 10 days. In certain embodiments, the immunoconjugate has a serum half-life of at least about 1 day, about 2 days, about 3 days, about 4 days, about 5 days, about 6 days, about 7 days, about 8 days, or about 9 days. In certain embodiments, the immunoconjugate has a serum half-life of at most about 2 days, about 3 days, about 4 days, about 5 days, about 6 days, about 7 days, about 8 days, about 9 days, or about 10 days.

In certain embodiments, the heavy chain constant region has a molecular weight of about 10 kDa to about 25 kDa. In certain embodiments, the heavy chain constant region has a molecular weight of about 10 kDa to about 15 kDa, about 10 kDa to about 20 kDa, about 10 kDa to about 25 kDa, about 15 kDa to about 20 kDa, about 15 kDa to about 25 kDa, or about 20 kDa to about 25 kDa. In certain embodiments, the heavy chain constant region has a molecular weight of about 10 kDa, about 15 kDa, about 20 kDa, or about 25 kDa. In certain embodiments, the heavy chain constant region has a molecular weight of at least about 10 kDa, about 15 kDa, or about 20 kDa. In certain embodiments, the heavy chain constant region has a molecular weight of at most about 15 kDa, about 20 kDa, or about 25 kDa.

### Chelating Agents

As described herein a chelating agent can be coupled to the immunoconjugates, the antigen binding region/immunoglobulin heavy chain constant region molecules, the VHH antigen binding region/immunoglobulin heavy chain constant region molecules (wild type or variant), the VHH antigen binding region/immunoglobulin Fc molecules (wild type or variant). The chelating agent allows for the immunoconjugate to be loaded with an appropriate radioisotope, such as a beta emitter or an alpha emitter. The chelator can be coupled to the immunoconjugate by the antigen binding region, the heavy chain constant region, the immunoglobulin Fc region, or any combination thereof. Such coupling can suitably be by a covalent attachment to one or more amino acids of the immunoconjugate, the antigen binding region, the heavy chain constant region, the immunoglobulin Fc region, or any combination thereof.

In some embodiments, a chelating agent of the immunoconjugate is covalently linked to an antigen binding region, the heavy chain constant region, the immunoglobulin Fc region, or any combination thereof. In some embodiments, a chelating agent is covalently linked to the antigen binding region, the heavy chain constant region, the immunoglobulin Fc region, or any combination thereof directly (e.g., without the use of a spacer, stretcher or linker). In some embodiments the chelating agent is covalently linked to the antigen binding arm through a linker that is covalently linked to the chelating agent and covalently linked to the antigen binding arm. In some embodiments, the linker is hydrophilic (e.g., a PEG chain). In some embodiments, the linker is hydrophobic (e.g., an alkyl or alkene chain). Chelators may be linked or coupled to the immunoconjugates as described in Sadiki, A. et al. “Site-specific conjugation of native antibody.” Antibody Therapeutics 2020, 3, 271-284.

In some embodiments, the immunoconjugate is formed through the attachment of the chelator-linker in a site-specific manner, directed into a specific amino acid or glycan residue. In some embodiments, the site-specific conjugation involves directed functionalization of a specific lysine residue in the framework region with the chelator-linker. In other embodiments, this residue may be functionalized with a different reactive functional group which then reacts in a second step with chelator-linker to furnish the immunoconjugate. In some embodiments, this reactive functional group is thiopropionate.

In some embodiments, a non-native cysteine residue is engineered into the framework of the antibody as a site for thiol directed conjugation to furnish the immunoconjugate. In some embodiments, other non-native amino acids or an amino acid sequence is engineered into the framework to serve as the attachment site for the chelator-linker or for a secondary reactive group upon which the chelator-linker will be conjugated to furnish the immunoconjugate.

In some embodiments, a non-natural amino acid containing a cross-linking group is engineered into the framework for attachment of the chelator-linker. In some embodiments, this non-natural amino-acid contains an azide.

In some embodiments, the chelator-linker is attached to a glutamine residue through the action of a transglutaminase enzyme. In other embodiments, a secondary reactive group is attached by transglutaminase upon which the chelator-linker is added to furnish the immunoconjugate.

In some embodiments, the chelator-linker is attached by modifying one or more N-glycans with a reactive functional group through the action of a glycosidase, then conjugation of the chelator-linker to that site. In some embodiments, the glycan is modified through the action of P-galactosidase. In some embodiments, the glycan is modified with a glycoside that contains an azide for attachment of a properly functionalized chelator-linker.

In some embodiments, the immunoconjugate comprises more than one chelating agent, which are the same or different.

In some embodiments, an immunoconjugate having more than one chelating agent has more than one chelating agent attached to the same antigen binding arm.

In some embodiments, an immunoconjugate having more than one chelating agent and less than eleven chelating agents has more than two chelating agents, more than three chelating agents, more than four chelating agents, more than five chelating agents, more than six chelating agents, more than seven chelating agents, more than eight chelating agents, or more than nine chelating agents. In some embodiments, the chelating agents are the same. In some embodiments, each antigen binding arm is linked directly or indirectly to more than one chelating agent.

In some embodiments, the chelating agent comprises a radioisotope chelating component and a functional group that allows for covalent attachment to the antigen binding arm. In some embodiments, the functional group is directly attached to the radioisotope chelating component. In some embodiments the chelating agent further comprises a linker between the functional group and the radioisotope chelating component.

In some embodiments, the radioisotope chelating component comprises DOTA or a DOTA derivative. In some embodiments, the radioisotope chelating component comprises DOTAGA. In some embodiments, the radioisotope chelating component comprises macropa or a macropa derivative. In some embodiments, the radioisotope chelating component comprises Py4Pa or a Py4Pa derivative.

In a preferred embodiment, the chelating agent of an immunoconjugate is not attached to the antigen binding region in the antigen binding arm of the immunoconjugate.

In some embodiments, the chelating agent of the immunoconjugate is non-covalently associated with an antigen binding arm. In a preferred embodiment, the chelator is not associated with the antigen binding region in the antigen binding arm of the immunoconjugate.

In some embodiments, the chelating agent comprises DOTA or a DOTA derivative. In some embodiments, the chelating agent comprises DOTAGA. In some embodiments, the chelating agent comprises macropa or a macropa derivative. In some embodiments, the chelating agent comprises Py4Pa or a Py4Pa derivative. In some embodiments, the chelating agent comprises siderocalin or a siderocalin derivative.

In certain embodiments, described herein is an immunoconjugate coupled to a chelating agent. In certain embodiments, the chelating agent is a radioisotope chelating agent. In certain embodiments, the radioisotope chelating agent is selected from the list consisting of: tetraazacyclododecane-1, 4, 7, 10-tetraacetic acid (DOTA), a-(2-Carboxyethyl) 1, 4, 7, 10-tetraazacyclododecane-1, 4, 7, 10-tetraacetic acid (DOTAGA), or (Py4Pa). In certain embodiments, the radioisotope chelating agent is DOTA. In certain embodiments, the radioisotope chelating agent is DOTAGA. In certain embodiments, the radioisotope chelating agent is Py4Pa. In certain embodiments, the radioisotope wherein the radioisotope chelating agent is directly coupled to the antigen binding region and/or the immunoglobulin heavy chain constant region. In certain embodiments, the radioisotope chelating agent is coupled to the antigen binding region or the immunoglobulin heavy chain constant region by a linker. In certain embodiments, the linker is selected from: 6-maleimidocaproyl (MC), maleimidopropanoyl (MP), valine-citrulline (val-cit), alanine-phenylalanine (ala-phe), p-amino-benzyl-oxycarbonyl (PAB), and those resulting from conjugation with linker reagents: N-Succinimidyl 4-(2-pyridylthio) pentanoate forming linker moiety 4-mercapto-pentanoic acid (SPP), Succinimidyl 4-(N-maleimidomethyl)cyclohexane-1-carboxylate (SMCC), N-Succinimidyl 4-(2-pyridyldithio)butanoate (SPDB), N-Succinimidyl (4-iodo-acetyl) aminobenzoate (SIAB), polyethylene glycol (PEG), a polyethylene glycol polymers (PEGn), and S-2-(4-Isothiocyanatobenzyl) (SCN). In certain embodiments, the linker is selected from: polyethylene glycol (PEG), a polyethylene glycol polymers (PEG), and S-2-(4-isothiocyanatobenzyl) (SCN). In certain embodiments, the linker is PEG5. In certain embodiments, the linker is SCN. In certain embodiments, the radioisotope chelating agent is a linker-chelator selected from the list consisting of: TFP-Ad-PEG5-DOTAGA, p-SCN-Bn-DOTA, p-SCN-Ph-Et-Py4Pa, and TFP-Ad-PEG5-Ac-Py4Pa.

The chelator may be conjugated at a ratio of protein or antigen binding region and/or the immunoglobulin heavy chain constant. In certain embodiments, the radioisotope chelating agent is coupled to the antigen binding region and/or the immunoglobulin heavy chain constant region at a ratio of 1:1 to 8:1. In certain embodiments, the radioisotope chelating agent is coupled to the antigen binding region and/or the immunoglobulin heavy chain constant region at a ratio of 1:1 to 6:1. In certain embodiments, the radioisotope chelating agent is coupled to the antigen binding region and/or the immunoglobulin heavy chain constant region at a ratio of 2:1 to 6:1.

In some embodiments, the immunoconjugate of the present invention comprises a linker, such as, e.g., to join an antigen binding arm to a chelating agent (interchangeably, “chelator”) or to a radioisotope or to cargo (e.g., a cytotoxin). A linker may comprise one or more linker components. In some embodiments, the immunoconjugate of the invention is engineered to have a terminal lysine available for conjugation to the chelating agent or linker.

For example, a bifunctional chelator is used to conjugate a radioisotope to a radioisotope delivery platform of the invention to create an immunoconjugate of the invention. (See e.g., Scheinberg D, McDevitt M, Curr Radiopharm 4: 306-20 (2011)). Examples of bifunctional chelators known in the art include DOTA, DTPA, DO3A-NHS, DOTAGA-NHS, DOTAGA-anhydride DOTAGA-TFP, p-SCN-Bn-DOTA, p-SCN-Bn-DTPA, p-SCN-Bn-CHX′A″-DTPA, p-SCN-Bn-TCMC, macropa-NCS, crown, p-SCN-Ph-Et-Py4Pa, 3, 2-HOPO, and TCMC.

Examples of bifunctional chelators are 1, 4, 7, 10-tetra-azacyclododecane-1, 4, 7, 10-tetraacetic acid (DOTA), diethylene triamine penta-acetic acid (DTPA), and related analogs of the aforementioned. Such chelators are suitable for coordinating metal ions like a and P-emitting radionuclides.

In some embodiments the chelating agent of an immunoconjugate or radioimmunoconjugate of the invention is selected from the group comprising bifunctional chelator, DOTA, DO3A-NHS, DOTAGA-NHS, DOTAGA-anhydride DOTAGA-TFP, p-SCN-Bn-DOTA, p-SCN-Bn-DTPA, p-SCN-Bn-CHX-A″-DTPA, p-SCN-Bn-TCMC, macropa-NCS (Thiele N A, et al. Angew. Chem. Int. Ed. 56:1 (2017)), crown (Yang H, et al. Chem. Eur. J. 26:11435 (2020)), P-SCN-Ph-Et-Py4Pa (Li L, et al. Bioconjugate Chem. ASAP (2020)), 3, 2-HOPO (Wickstroem K, et al. Int. J. Rad. Onc. Biol. Phys. 105:410 (2019)) (For a review of these and other bifunctional chelators See e.g., Price EW and Orvig C Chem. Soc. Rev., 2014, 43:260 (2014) and Brechbiel MW Q. J. Nucl. Med. Mol. Imaging 52:166 (2008)).

In some embodiments the chelating agent of an immunoconjugate or radioimmunoconjugate of the invention is selected from the group consisting of bifunctional chelator, DOTA, DO3A-NHS, DOTAGA-NHS, DOTAGA-anhydride DOTAGA-TFP, p-SCN-Bn-DOTA, p-SCN-Bn-DTPA, p-SCN-Bn-CHX-A″-DTPA, p-SCN-Bn-TCMC, macropa-NCS (Thiele N A, et al. Angew. Chem. Int. Ed. 56:1 (2017)), crown (Yang H, et al. Chem. Eur. J. 26:11435 (2020)), P-SCN-Ph-Et-Py4Pa (Li L, et al. Bioconjugate Chem. ASAP (2020)), 3, 2-HOPO (Wickstroem K, et al. Int. J. Rad. Onc. Biol. Phys. 105:410 (2019)) (For a review of these and other bifunctional chelators see e.g., Price EW and Orvig C Chem. Soc. Rev., 2014, 43:260 (2014) and Brechbiel MW Q. J. Nucl. Med. Mol. Imaging 52:166 (2008)).

For 225-Ac immunoconjugates, there are a variety of acyclic and cyclic ligands known in the art as suitable chelators (see e.g., Davis I, et al., Nucl Med Biol 26: 581 (1999); Chappell L, et al., Bioconjug Chem 11: 510 (2000); Chappell, L, et al., Nucl Med Biol 30: 581 (2003); McDevitt M, et al., Appl Radiat Isot 57: 841 (2002); Gouin S, et al., Org Biomol Chem 3: 453 (2005); Thiele N, et al., Angew Chem Int Ed Engl 56: 14712 (2017)).

In certain embodiments, the chelator is a chelator suitable for alpha emitter chelation. Some chelators suitable for alpha emitters are described in Yang et al, “Harnessing a-Emitting Radionuclides for Therapy: Radiolabeling Method Review.” J Nucl Med. 2022 January; 63(1):5-13.

In certain embodiments the, chelator suitable for alpha emitter chelation is selected from the list consisting of: DOTA 1, 4, 7, 10-tetra-azacyclododecane-1, 4, 7, 10-tetraacetic acid; DO3A 1, 4, 7-Tris(carboxy-methyl) 1, 4, 7, 10-tetraazacyclododecane; DOTAGA a-(2-Carboxyethyl) 1, 4, 7, 10-tetra-azacyclododecane-1, 4, 7, 10-tetraacetic acid; DOTAGA anhydride (2, 2′, 2″-(10-(2, 6-dioxotetrahydro-2H-pyran-3-yl) 1, 4, 7, 10-tetraazacyclododecane-1, 4, 7-triyl)triacetic acid; Py4Pa 6, 6′, 6″, 6′″-(((pyridine-2, 6-diylbis(methylene)) bis(azanetriyl))tetrakis(methylene)) tetrapicolinic acid; Py4Pa-NCS is 6, 6′-((((4-isothiocyanatopyridine-2, 6-diyl)bismethylene))bis((carboxymethyl)azanediyl))bis-(methylene))dipicolinic acid; Crown 2, 2′, 2″, 2′″-(1, 10-dioxa-4, 7, 13, 16-tetra-azacyclooctadecane-4, 7, 13, 16-tetrayl) tetra-acetic acid; Macropa 6, 6′-((1, 4, 10, 13-tetraoxa-7, 16-diazacyclooctadecane-7, 16-diyl) bis(methylene)) dipicolinic acid; Macropa-NCS 6-((16-((6-carboxypyridin-2-yl)methyl) 1, 4, 10, 13-tetraoxa-7, 16-diazacyclooctadecan-7-yl)methyl) 4-isothiocyanatopicolinic acid; HEHA 1, 4, 7, 10, 13, 16-hexaazacyclohexadecane-1, 4, 7, 10, 13, 16-hexaacetic acid; CHX octapa 6, 6′-[(1R, 2R) 1, 2-Cyclohexanediylbis-[[(carboxy-methyl)imino]methylene]]bis[2-pyridinecarboxylic acid]; Bispa 3, 7-Diazabicyclo[3.3.1]nonane-1, 5-dicarboxylic acid, 7-[(6-carboxy-2-pyridinyl)methyl]-9-hydroxy-3-methyl-2, 4-di-2-pyridinyl-, 1, 5-dimethyl ester; Noneunpa 6, 6′-(((oxybis(ethane-2, 1-diyl)) bis((carboxymethyl)azanediyl))bis(methylene))dipicolinic acid; and combinations thereof.

In certain embodiments, the chelator is a chelator suitable for a beta- or gamma-emitter chelation. In certain embodiments the, chelator suitable for a beta- or gamma-emitter chelation is selected from the list consisting of. DOTMA (1R, 4R, 7R, 1OR) a, a′, a″, a′″-tetramethyl-1, 4, 7, 10-tetraazacyclododecane-1, 4, 7, 1 0-tetraacetic acid DOTAM (1, 4, 7, 10-tetrakis(carbamoylmethyl) 1, 4, 7, 10-tetraazacyclododecane); DOTPA 1, 4, 7, 10-tetraazacyclododecane-1, 4, 7, 10-tetra propionic acid; DO3AM-acetic acid (2-(4, 7, 10-tris(2-amino-2-oxoethyl) 1, 4, 7, 10-tetraazacyclododecan-1-yl)acetic acid); DOTP 1, 4, 7, 10-tetraazacyclododecane-1, 4, 7, 10-tetra(methylene phosphonic acid); DOTMP 1, 4, 6, 10-tetraazacyclodecane-1, 4, 7, 10-tetramethylene phosphonic acid; DOTA-4AMP 1, 4, 7, 10-tetraazacyclododecane-1, 4, 7, 10-tetrakis(acetamido-methylenephosphonic acid); CB-TE2A (1, 4, 8, 11-tetraazabicyclo [6.6.2]hexadecane-4, 11-diacetic acid); NOTA 1, 4, 7-triazacyclononane-1, 4, 7-triacetic acid; NOTP 1, 4, 7-triazacyclononane-1, 4, 7-tri(methylene phosphonic acid); TETPA 1, 4, 8, 11-tetraazacyclotetradecane-1, 4, 8, 11-tetrapropionic acid; TETA 1, 4, 8, 11-tetraazacyclotetradecane-1, 4, 8, 11-tetraacetic acid; PEPA 1, 4, 7, 10, 13-pentaazacyclopentadecane-N, N′, N″, N′″, N″″-pentaacetic acid; H4Octapa N, N′-bis(6-carboxy-2-pyridylmethyl) ethylenediamine-N, N′-diacetic acid; H2Dedpa 1, 2-[[6-(carboxy) pyridin-2-yl]-methylamino]ethane; H6phospa N, N′-(methylene-phosphonate) N, N′-[6-(methoxycarbonyl)pyridin-2-yl]-methyl-1, 2-diaminoethane; TTHA triethylenetetramine-N, N, N′, N″, N′″, N′″-hexaacetic acid; DO2P tetraazacyclododecane dimethanephosphonic acid; HP-DO3A hydroxy-propyltetraazacyclododecanetriacetic acid; EDTA ethylenediaminetetraacetic acid; DTPA diethylenetriaminepentaacetic acid; DTPA-BMA diethylenetriamine-pentaacetic acid-bismethylamide; HOPO octadentate hydroxypyridinones; 3, 2, 3-LI(HOPO) N, N′-(butane-1, 4-diyl)bis(1-hydroxy-N-(3-(1-hydroxy-6-oxo-1, 6-dihydropyridine-
2-carboxamido)propyl) 6-oxo-1, 6-dihydropyridine-2-carboxamide); 3, 2-HOPO N, N′
-(((2-(4-aminobenzyl) 3-((2-(3-hydroxy-1-methyl-2-oxo-1, 2-dihydro-pyridine-4-carboxamido)ethyl)(2-(3-hydroxy-2-oxo-1, 2-dihydropyridine-4-carbox-amido)ethyl)amino)propyl)azanediyl)bis(ethane-2, 1-diyl))bis(3-hydroxy-1-methyl-2-oxo-1, 2-dihydropyridine-4-carboxamide); Neunpa 6, 6′-(((azanediylbis(ethane-2, 1-diyl))bis((carboxymethyl)azanediyl))bis(methylene))dipicolinic acid; Neunpa-NCS=6, 6′-(((((4-isothiocyanatophenethyl)azanediyl)bis(ethane-2, 1-diyl))bis ((carboxymethyl)azanediyl))bis(methylene))dipicolinic acid; Octapa 6, 6′-((ethane-1, 2-diylbis((carboxymethyl)azanediyl))bis(methylene))dipicolinic acid; Octox 2, 2′-(ethane-1, 2-diylbis(((8-hydroxyquinolin-2-yl)methyl)azanediyl))diacetic acid; PyPa 6, 6′-(((pyridine-2, 6-diylbis(methylene))bis((carboxymethyl)azanediyl))bis(methyl-ene)) dipicolinic acid; Porphyrin 21, 22, 23, 24-Tetraazapentacyclo [16.2.1.13, 6.18, 11.113, 16]tetracosa-1, 3, 5, 7, 9, 11(23), 12, 14, 16, 18(21), 19-undecaene; Deferoxamine 30-Amino-3, 14, 25-trihydroxy-3, 9, 14, 20, 25-penta-azatriacontane-2, 10, 13, 21, 24-pentaone; DFO* N1-[5-(Acetyl-hydroxyamino) pentyl]-N26-(5-aminopentyl) N26, 5, 16-trihydroxy-4, 12, 15, 23-tetraoxo-5, 11, 16, 22-tetra-azahexacosanediamide; and combinations thereof.

Alternatively, or in addition, an isothiocyanate linker may be used, such as p-SCN-Bn-DOTA, involving a lysine residue within an immunoconjugate of the invention.

Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), and those resulting from conjugation with linker reagents: N-Succinimidyl 4-(2-pyridylthio) pentanoate forming linker moiety 4-mercaptopentanoic acid (“SPP”), N-succinimidyl 4-(N-maleimidomethyl) cyclohexane-1 carboxylate forming linker moiety 4-((2, 5-dioxopyrrolidin-1-yl)methyl)cyclohexanecarboxylic acid (“SMCC”, also referred to herein as “MCC”), 2, 5-dioxopyrrolidin-1-yl 4-(pyridin-2-yldisulfanyl) butanoate forming linker moiety 4-mercaptobutanoic acid (“SPDB”), N-Succinimidyl (4-iodo-acetyl) aminobenzoate (“SIAB”), ethyleneoxy —CH2CH2O— as one or more repeating units (“EO, ““PEO, “or “PEG”). Additional linker components are known in the art and some are described herein. Various linker components are known in the art, some of which are described below.

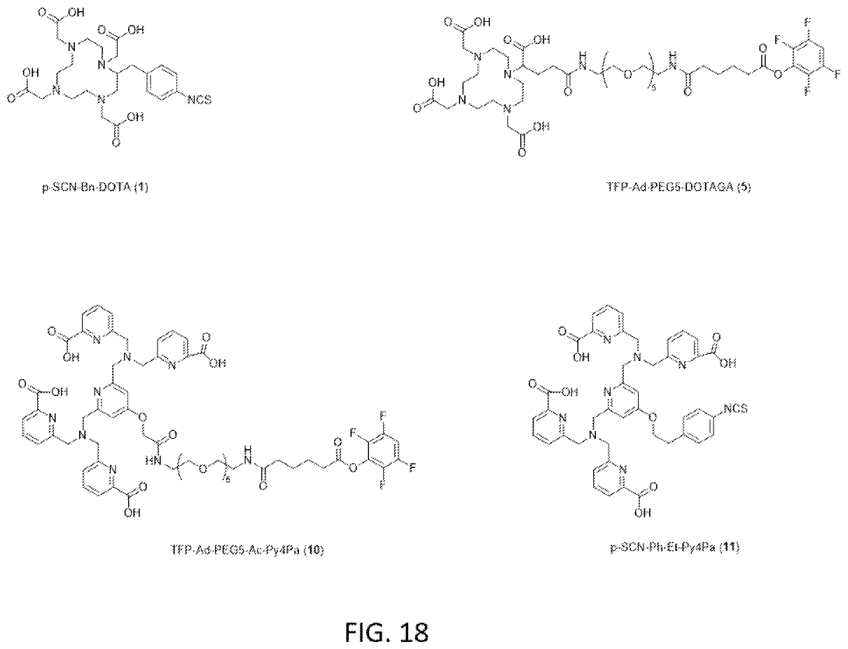

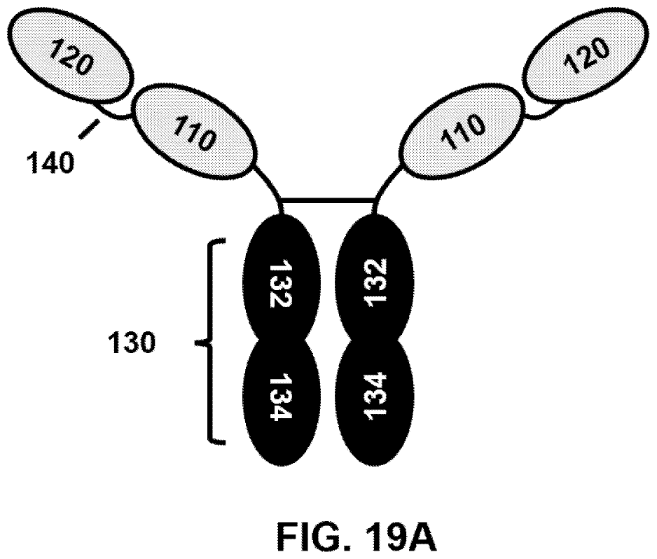

In certain embodiments, the linker is SCN. In certain embodiments, the chelating agent is a linker-chelator selected from the list consisting of: TFP-Ad-PEG5-DOTAGA, p-SCN-Bn-DOTA, p-SCN-Ph-Et-Py4Pa, and TFP-Ad-PEG5-Ac-Py4Pa. In certain embodiments, the chelating agent is TFP-Ad-PEG5-DOTAGA. In certain embodiments, the chelating agent is p-SCN-Bn-DOTA. In certain embodiments, the chelating agent is p-SCN-Ph-Et-Py4Pa. In certain embodiments, the chelating agent is TFP-Ad-PEG5-Ac-Py4Pa. Such linkers are shown in FIG. 18.

A linker may be a “cleavable linker, “facilitating release of a drug in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-31 (1992); U.S. Pat. No. 5,208,020) may be used.

In certain embodiments, a linker is as shown in the following formula:

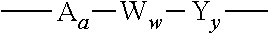

wherein A is a stretcher unit, and a is an integer from 0 to 1; W is an amino acid unit, and w is an integer from 0 to 12; Y is a spacer unit, and y is 0, 1, or 2; and Ab, D, and p are defined as above. Exemplary embodiments of such linkers are described in US 20050238649.

In some embodiments, a linker component may comprise a “stretcher unit” that links an immunoconjugate to another linker component or to a drug moiety. Exemplary stretcher units are shown below (wherein the wavy line indicates sites of covalent attachment to an immunoconjugate):

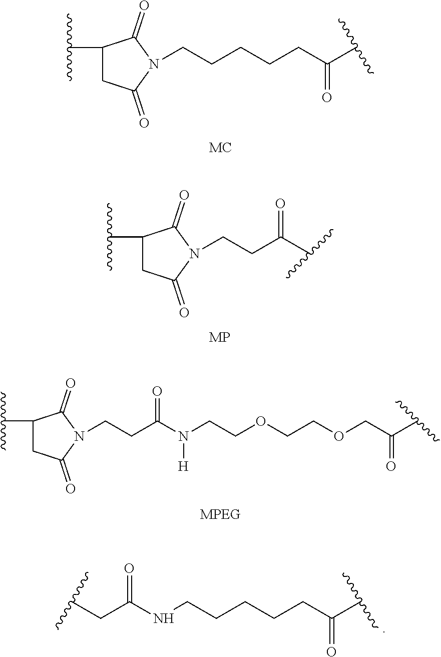 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker may be conjugated to an antibody through a cysteine bridging functionality such as ThioBridge® or DBM (dibromomaleimide). These linkers can act to restabilize intrachain disulfides after reduction and conjugation (Bird M, et al., Antibody-Drug Conjugates pp. 113-129 (2019) and Behrens C R, et al. Mol. Pharmaceutics 12:3986 (2015)). Exemplary rebridging stretcher elements are shown below (wherein the wavy line indicates sites of covalent attachment to an immunoconjugate):

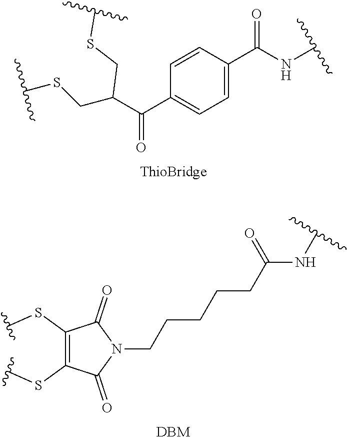 [SMILES: *NC(=O)c1ccc(C(=O)C(CS*)CS*)cc1 ; *NC(=O)CCCCCN1C(=O)C(S*)=C(S*)C1=O]

In some embodiments, a linker component may comprise an amino acid unit. In one such embodiment, the amino acid unit allows for cleavage of the linker by a protease, thereby facilitating release of the drug from the immunoconjugate upon exposure to intracellular proteases, such as lysosomal enzymes (see, e.g., Doronina et al. (2003) Nat. Biotechnol. 21: 778-4. Exemplary amino acid units include, but are not limited to, a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys); or N-methyl-valine-citrulline (Me-val-cit). Exemplary tripeptides include: glycine-valine-citrulline (gly-val-cit) and glycine-glycine-glycine (gly-gly-gly). An amino acid unit may comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, a linker component may comprise a “spacer” unit that links the immunoconjugate to a drug moiety, either directly or by way of a stretcher unit and/or an amino acid unit. A spacer unit may be “self-immolative” or a “non-self-immolative.” A “non-self-immolative” spacer unit is one in which part or all of the spacer unit remains bound to the drug moiety upon enzymatic (e.g., proteolytic) cleavage of the ADC. Examples of non-self-immolative spacer units include, but are not limited to, a glycine spacer unit and a glycine-glycine spacer unit. Other combinations of peptidic spacers susceptible to sequence-specific enzymatic cleavage are also contemplated. For example, enzymatic cleavage of an ADC containing a glycine-glycine spacer unit by a tumor-cell associated protease would result in release of a glycine-glycine-drug moiety from the remainder of the ADC. In one such embodiment, the glycine-glycine-drug moiety is then subjected to a separate hydrolysis step in the tumor cell, thus cleaving the glycine-glycine spacer unit from the drug moiety.

A “self-immolative” spacer unit allows for release of the drug moiety without a separate hydrolysis step. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent (see, e.g., Hamann et al. (2005) Expert Opin. Ther. Patents (2005) 15: 1087-103. In some embodiments, the spacer unit is p-aminobenzyloxycarbonyl (PAB). In certain embodiments, the phenylene portion of a p-amino benzyl unit is substituted with Qm, wherein Q is —C1-C8 alkyl, —O—(C1-C8 alkyl), -halogen, -nitro or -cyano; and m is an integer ranging from 0-4. Examples of self-immolative spacer units further include, but are not limited to, aromatic compounds that are electronically similar to p-aminobenzyl alcohol (see, e.g., US 2005/0256030 A1), such a s 2 to aminoimidazol-5-methanol derivatives (Hay et al. (1999) Bioorg. Med. Chem. Lett. 9: 2237) and ortho- or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223); appropriately substituted bicyclo[2.2.1] and bicyclo[2.2.2] ring systems (Storm, et al., J. Amer. Chem. Soc., 1972, 94: 5815); and 2-aminophenylpropionic acid amides (Amsberry, et al., J. Org. Chem., 1990, 55: 5867). Elimination of amine-containing drugs that are substituted at the a-position of glycine (Kingsbury, et al., J. Med. Chem., 1984, 27: 1447) are also examples of self-immolative spacers useful in ADCs.

In some embodiments, a spacer unit is a branched bis(hydroxymethyl)styrene (BHMS) unit as depicted below, which can be used to incorporate and release multiple drugs.

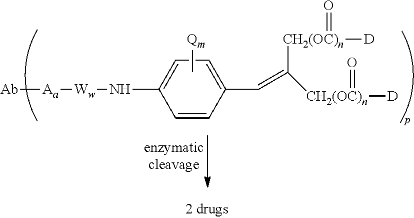

wherein Q is —C1-C8 alkyl, —O—(C1-C8 alkyl), -halogen, -nitro or -cyano; m is an integer ranging from 0-4; n is 0 or 1; and p ranges ranging from 1 to about 20.

In some embodiments, the immunoconjugate comprises a linker, such as, e.g., a dendritic type linker for covalent attachment of more than one drug moiety through a branching, multifunctional linker moiety to an antibody (Sun et al (2002) Bioorganic & Medicinal Chemistry Letters 12: 2213-5; Sun et al (2003) Bioorganic & Medicinal Chemistry 11: 1761-8). Dendritic linkers can increase the molar ratio of drug to antibody, i.e. loading, which is related to the potency of the ADC. Thus, where a cysteine-engineered antibody bears only one reactive cysteine thiol group, a multitude of drug moieties may be attached through a dendritic linker.

Examples of linker components and combinations thereof are shown below, which are also suitable for use in the formula above:

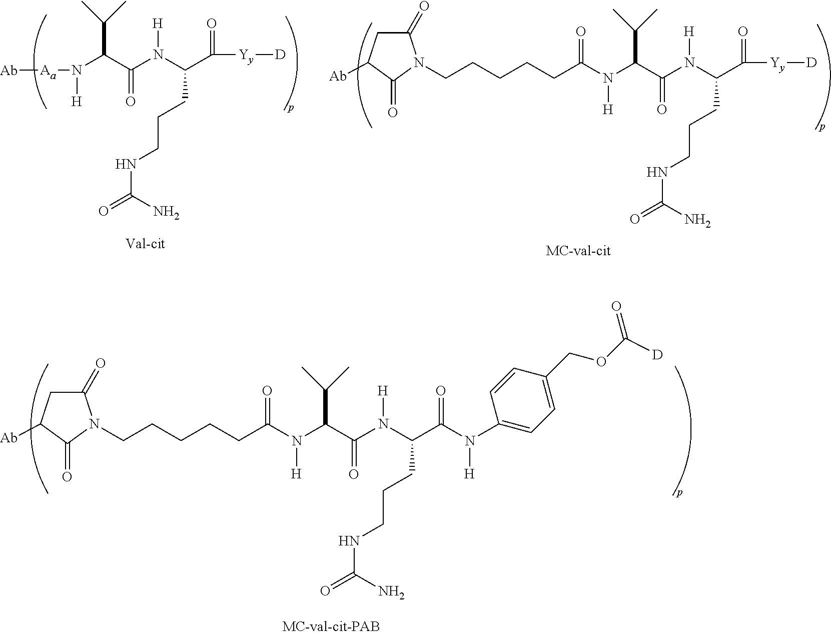

Additional non-limiting examples of linkers include those described in WO 2015095953.

Linkers components, including stretcher, spacer, and amino acid units, may be synthesized by methods known in the art, such as those described in US 20050238649.

In some embodiments the chelating agent comprises a linker and is selected from and one of the compounds described in U.S. Application No. 63/373,189, filed Aug. 22, 2022, or a U.S. non-provisional application or international application claiming priority thereto, which are hereby incorporated by reference for such compounds. In some embodiments the chelating agent comprises a linker and is selected from: Compound 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, and 1-34 of U.S. Application No. 63/373,189, filed Aug. 22, 2022, which is hereby incorporated by reference for such compounds.

In some embodiments the chelating agent comprises a linker and is selected from and one of the compounds described in U.S. Application No. 63/373,183, filed Aug. 22, 2022, or a U.S. non-provisional application or international application claiming priority thereto, which are hereby incorporated by reference for such compounds. In some embodiments the chelating agent comprises a linker and is selected from: Compound 2-1, 2-2, 2-3, 2-4, 2-5, 2-6, 2-7, 2-8, 2-9, 2-10, 2-11, 2-12, and 2-13 of U.S. Application No. 63/373,183, filed Aug. 22, 2022, which is hereby incorporated by reference for such compounds.

In some embodiments the chelating agent comprises a linker and is selected from and one of the compounds described in U.S. Application No. 63/373,190, filed Aug. 22, 2022, or a U.S. non-provisional application claiming priority thereto, which are hereby incorporated by reference for such compounds. In some embodiments the chelating agent comprises a linker and is selected from: Compound 3-1, 3-2, 3-3, 3-4, 3-5, 3-13, 3-16 of U.S. Application No. 63/373,190, filed Aug. 22, 2022, which is hereby incorporated by reference in its entirety for such compounds.

### Radio-Immunoconjugates

A “radionuclide” or “radioisotope” refers to and encompasses an alpha emitting isotope (interchangeably, α-emitting isotope), beta-emitting isotope (interchangeably, β-emitting isotope), and/or gamma-emitting isotope (interchangeably, γ-emitting isotope), such as, e.g., any one of 86-Y, 90-Y, 177-Lu, 186-Re, 188-Re, 89-Sr, 153-Sm, 225-Ac, 213-Bi, 213-Po, 212-Bi, 223-Ra, 224-Ra, 227-Th, 149-Tb, 68-Ga, 64-Cu, 67-Cu, 89-Zr, 137-Cs, 212-Pb, and 103-Pd.

A “radio-immunoconjugate” (also used interchangeable with “immunoconjugate” when used in a treatment) refers to and encompasses a molecular complex comprising (1) an immunoconjugate according to the present invention and (2) a radioisotope. In some embodiments, the radioisotope is an α-emitting radioisotope. In some embodiments, the radioisotope is a β-emitting radioisotope. In some embodiments, the radioisotope is a γ-emitting isotope. In some embodiments, the invention provides radioimmunoconjugates comprising α-emitting and β-emitting radioisotopes. The term “radioconjugate” is alsoused interchangeably with the term “radioimmunoconjugate” herein. In some embodiments, the radioisotope is associated with a chelating agent of the radioimmunoconjugate. In some embodiments, the radioisotope is directly linked to the immunoconjugate.

In some embodiments, the invention provides immunoconjugates. In some embodiments, the immunoconjugates are capable of delivering α-emitters in vivo when so labeled, linked or loaded with an α-emitter. In some embodiments, the immunoconjugates are also capable of delivering other radioisotopes (β-emitters, and/or γ-emitters), and/or other atoms in vivo, when so labeled, linked or loaded. In some embodiments, the immunoconjugates are capable of delivering imaging metals (e.g., 111-In, 89-Zr, 64-Cu, 68-Ga or 134-Ce) in vivo when so labeled, linked or loaded.

The immunoconjugates of the current disclosure may be loaded with a radioisotope for a therapeutic or diagnostic effect. In certain embodiments, the chelator may further comprise a radioisotope. In certain embodiments, the radioisotope is an alpha emitter. In certain embodiments, the radioisotope is an alpha emitter selected from the list consisting of 225-Ac, 223-Ra, 224-Ra, 227-Th, 212-Pb, 212-Bi, and 213-Bi. In certain embodiments, the radioisotope is 225-Ac. In certain embodiments, the radioisotope is a beta emitter. In certain embodiments, the radioisotope is a beta emitter selected from 177-Lu, 90-Y, 67-Cu, and 153-Sm.

Also described herein is a method of making a radioimmunoconjugate comprising loading or complexing an immunoconjugate of the current disclosure to a radioisotope. In certain embodiments, the radioisotope is an alpha emitter. In certain embodiments, the radioisotope is an alpha emitter selected from the list consisting of 225-Ac, 223-Ra, 224-Ra, 227-Th, 212-Pb, 212-Bi, and 213-Bi. In certain embodiments, the radioisotope is 225-Ac. In certain embodiments, the radioisotope is a beta emitter. In certain embodiments, the radioisotope is a beta emitter selected from 177-Lu, 90-Y, 67-Cu, and 153-Sm.

In some embodiments, the invention provides a radioimmunoconjugate, comprising an immunoconjugate of the invention and an α-emitting radioisotope. In some embodiments, the α-emitting radioisotope of the radioimmunoconjugate is selected from the group comprising: 225-Ac, 223-Ra, 224-Ra, 227-Th, 212-Pb, 212-Bi, and 213-Bi. In some embodiments, the α-emitting radioisotope of the radioimmunoconjugate is selected from the group consisting of: 225-Ac, 223-Ra, 224-Ra, 227-Th, 212-Pb, 212-Bi, and 213-Bi. In some embodiments, the α-emitting radioisotope of the radioimmunoconjugate is 225-Ac. In some embodiments, the α-emitting radioisotope of the radioimmunoconjugate is 223-Ra. In some embodiments, the α-emitting radioisotope of the radioimmunoconjugate is 224-Ra. In some embodiments, the α-emitting radioisotope of the radioimmunoconjugate is 227-Th. In some embodiments, the α-emitting radioisotope of the radioimmunoconjugate is 212-Pb. In some embodiments, the α-emitting radioisotope of the radioimmunoconjugate is 212-Bi. In some embodiments, the α-emitting radioisotope of the radioimmunoconjugate is 213-Bi.

In some embodiments, the immunoconjugate of the present invention is combined with a radioisotope to provide a radioimmunoconjugate of the invention. In some embodiments, the radioisotope is 225-Ac, 86-Y, 90-Y, 177-Lu, 186-Re, 188-Re, 89-Sr, 153-Sm, 213-Bi, 213-Po, 212-Bi, 223-Ra, 224-Ra, 227-Th, 149-Tb, 68-Ga, 64-Cu, 67-Cu, 89-Zr, 137-Cs, 212-Pb, or 103-Pd. In some embodiments, the radioisotope is an alpha emitter, such as, e.g., 225-Ac, 223-Ra, 224-Ra, 227-Th, 212-Pb, 212-Bi, and 213-Bi. In some embodiments, the radioisotope is a beta particle emitter, such as, e.g., 177-Lu, 90-Y, 67-Cu, 153-Sm. In some embodiments, the radioisotope is both an alpha particle emitter and a beta and/or gamma particle emitter. In some embodiments, the radioisotope is both a beta particle emitter and a gamma particle and/or photon emitter. In some embodiments, the radioimmunoconjugate is labeled, linked or loaded with, and accordingly comprises, both an α-emitter and a β-emitter. In some embodiments, the radioisotope is selected for use in radio-imaging, such as, e.g., from among 68-Ga, 64-Cu, 89-Zr, 111-In, 134-Ce.

The immunoconjugates and radioimmunoconjugates of the invention may comprise other cargos or payloads besides a radioisotope, including various cytotoxic agents, such as, e.g., a small molecule chemotherapeutic agent, cytotoxic antibiotic, alkylating agent, antimetabolite, topoisomerase inhibitor, and/or tubulin inhibitor. For example, an immunoconjugate of the invention may be used to deliver a non-radioisotope cytotoxin to a target cell. Non-limiting examples of cytotoxic agents include aziridines, cisplatins, tetrazines, procarbazine, hexamethyl-lmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolo-benzodiazepine, carboplatin, 5-fluorouracil (5-FU), capecitabine, mitomycin C, paclitaxel, 1,3-Bis(2-chloroethyl) 1-nitrosourea (BCNU), rifampicin, cisplatin, methotrexate, and gemcitabine.

In some embodiments, a radioimmunoconjugate of the invention comprises a radioisotope selected from the group comprising 225-Ac, 86-Y, 90-Y, 177-Lu, 186-Re, 188-Re, 89-Sr, 153-Sm, 213-Bi, 213-Po, 211-At, 212-Bi, 223-Ra, 224-Ra, 227-Th, 149-Tb, 68-Ga, 64-Cu, 67-Cu, 89-Zr, 137-Cs, 212-Pb, and 103-Pd.

In some embodiments, a radioimmunoconjugate of the invention comprises a radioisotope selected from the group consisting of 225-Ac, 86-Y, 90-Y, 177-Lu, 186-Re, 188-Re, 89-Sr, 153-Sm, 213-Bi, 213-Po, 211-At, 212-Bi, 223-Ra, 224-Ra, 227-Th, 149-Tb, 68-Ga, 64-Cu, 67-Cu, 89-Zr, 137-Cs, 212-Pb, and 103-Pd.

In some embodiments, the radioisotope is an alpha-particle-emitting radioisotope comprises 225-Ac, 223-Ra, 224-Ra, 227-Th, 212-Pb, 212-Bi, or 213-Bi.

In some embodiments, the radioisotope is an alpha-particle-emitting radioisotope selected from the group consisting of 225-Ac, 223-Ra, 224-Ra, 227-Th, 212-Pb, 212-Bi, and 213-Bi.

### Immunoconjugate Derivatives and Other Modifications

Covalent modifications of the immunoconjugates of the invention are included within the scope of this invention. One type of covalent modification includes reacting targeted amino acid residues of an immunoconjugate of the invention with an organic derivatizing agent that is capable of reacting with selected side chains or the N- or C-terminal residues of the immunoconjugate. Derivatization with bifunctional agents is useful, for instance, for crosslinking an immunoconjugate of the invention to a water-insoluble support matrix or surface for use in the method for purifying the immunconjugates of the invention, and vice-versa. Commonly used crosslinking agents include, e.g., 1,1-bis(diazoacetyl) 2-phenylethane, glutar-aldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

In some embodiments, an immunoconjugate provided herein may be further modified to contain additional nonproteinaceous moieties that are known in the art and readily available. The moieties suitable for derivatization of the immunoconjugate include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1, 3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the immunoconjugate may vary, and if more than one polymer is attached, they can be the same or different molecules. In general, the number and/or type of polymers used for derivatization can be determined based on considerations including, but not limited to, the particular properties or functions of the immunoconjugate to be improved, whether the immunoconjugate derivative will be used in a therapy under defined conditions, etc.

PEG derivatized immunoconjugates of the invention may comprise linkers comprising one or more —CH₂CH₂O— and can be used to alter biodistribution and pharmacokinetics of the immunoconjugate. PEGs can be prepared in a polymeric form or as discrete oligomers. Bifunctionalized versions of these polymers can link immunoconjugatess with a chelating agent and/or provide additional size and/or solubility to the overall molecule. In some embodiments, the PEG derivatized immunoconjugates exhibit reduced immunogenicity compared to their un-derivatized parental molecules.

### Methods of Producing the Immunoconjugates

The present invention provides a composition comprising one or more of the immunoconjugates according to any of the above embodiments or described herein. In another embodiment, the invention provides an isolated nucleic acid encoding a radioisotope delivering platform as described herein. Also provided herein are nucleic acids encoding the protein components of the immunoconjugates of the present invention, expression vectors comprising the aforementioned nucleic acid, and host cells comprising the aforementioned expression vectors.

In another embodiment, the invention provides a host cell comprising a nucleic acid and/or vector as provided herein. In some embodiments, the host cell of the present invention is isolated or purified. In some embodiments, the host cell of the present invention is in a cell culture medium. The nucleic acids, expression vectors, and host cells of the invention may be used to produce a composition comprising one or more of the immunoconjugates of the invention. In some embodiments, the host cell is eukaryotic. In some embodiments, the host cell is mammalian. In some embodiments, the host cell is a Chinese Hamster Ovary (CHO) cell. In some embodiments, the host cell is prokaryotic. In some embodiments, the host cell is E. coli.

A description follows as to illustrative techniques for the production of the immunoconjugates and radioimmunoconjugates of the present invention for use in accordance with the methods of the present invention. In some embodiments, the invention provides a process for making an immunoconjugate of the present invention, the method comprising culturing a host cell as provided herein under conditions suitable for the expression vector encoding the radioisotope delivery platform and recovering or purifying the radioisotope delivery platform. In some embodiments, the method further comprises radiolabeling the radioisotope delivery platform with an appropriate isotope, such as, e.g., an alpha or beta particle emitter.

Generation and Identification of Antigen Binding Domains, Immunoconjugates and Nucleic Acids

Antigen binding domains useful as antigen binding regions herein may be identified in antibodies that are either monoclonal antibodies and/or polyclonal antibodies. DNA encoding a monoclonal antibody is readily isolated and sequenced using conventional procedures. Once isolated, the DNA may be placed into expression vectors, which are then transfected into host cells such as E. coli cells, simian COS cells, Chinese hamster ovary (CHO) cells, or myeloma cells that do not otherwise produce antibody protein, to obtain the synthesis of monoclonal antibodies in the recombinant host cells (see e.g., Skerra et al., Curr. Opinion in Immunol., 5:256-262 (1993) and Pluckthun, Immunol Revs. 130:151-188 (1992)).

In some embodiments, the antigen binding domains of an immunoconjugate of the present invention, or fragments thereof, are isolated by screening phage libraries containing phage that display various fragments of antibody variable region (Fv, scFv, or VHH) fused to phage coat protein. Such phage libraries are screened for binding to the desired target antigen or epitope. Clones expressing Fv fragments, scFv's, or VHH's capable of binding to the desired antigen are adsorbed to the antigen and thus separated from the non-binding clones in the library. The binding clones are then eluted from the antigen, and can be further enriched by additional cycles of antigen adsorption/elution.

In some embodiments, the antibody or antibody fragments thereof are isolated from antibody phage libraries generated using the techniques described in McCafferty et al., Nature, 348:552-554 (1990). Clackson et al., Nature, 352:624-628 (1991) and Marks et al., J Mol Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc Acids Res. 21:2265-2266 (1993)). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen binding clones as described in Winter et al., Ann. Rev. Immunol., 12: 433-455 (1994). Naïve libraries for screening can be constructed from non-immunized sources to provide high-affinity antibodies to antigens (see e.g., Griffiths et al., EMBO J, 12: 725-734 (1993)). Another example is naive libraries constructed synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

Screening of the libraries can be accomplished by various techniques known in the art. For example, target antigen can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (and strong binding affinities) can be promoted by use of long washes and monovalent phage display as described in Bass et al., Proteins, 8: 309-314 (1990) and in WO 1992/09690, and a low coating density of antigen as described in Marks et al., Biotechnol., 10: 779-783 (1992).

Techniques for screening a cDNA library are well known in the art. Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding immunoconjugate of the invention is to use PCR methodology (Sambrook et al., supra; Dieffenbach et al., PCR Primer: A Laboratory Manual (Cold Spring Harbor Laboratory Press, 1995)).

DNA encoding an immunoconjugate of the invention may be obtained from a cDNA library prepared from tissue believed to possess the immunoconjugate of the invention mRNA and to express it at a detectable level. Accordingly, human immunoconjugate of the invention DNA can be conveniently obtained from a cDNA library prepared from human tissue. The immunoconjugate of the invention-encoding gene may also be obtained from a genomic library or by known synthetic procedures (e.g., automated nucleic acid synthesis). For some embodiments, desired polynucleotide sequences encoding antibodies may be isolated and sequenced from antibody producing cells such as hybridoma cells.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein. Any of the antibody CDRs or heavy chain variable fragments of the present invention can be obtained by designing a suitable antigen screening procedure to select for the phage clone of interest followed by construction of an antibody clone using the variable domain and/or CDRs sequences from a phage clone of interest and suitable constant region (Fc) sequences described in Kabat et al., 1991, supra.

### Immunoconjugate Production; Host Cells and Expression Vectors of the Invention

The description below relates primarily to production of the antibody constructs of the invention by culturing cells transformed or transfected with a vector-containing immunoconjugate of the invention-encoding nucleic acid. It is, of course, contemplated that alternative methods, which are well known in the art, may be employed to prepare the antibody constructs of the invention. For instance, the appropriate amino acid sequence, or portions thereof, may be produced by direct peptide synthesis using solid-phase techniques (e.g., Stewart et al., Solid-Phase Peptide Synthesis, W.H. Freeman Co., San Francisco, CA (1969); Merrifield, J, Am. Chem. Soc., 85: 2149-54 (1963)). In vitro protein synthesis may be performed using manual techniques or by automation. Automated synthesis may be accomplished, for instance, using an Applied Biosystems Peptide Synthesizer (Foster City, CA) using manufacturer's instructions. Various portions of the immunoconjugate of the invention may be chemically synthesized separately and combined using chemical or enzymatic methods to produce the desired immunoconjugate of the invention.

Antibody constructs may be produced using recombinant methods and compositions, e.g., as described in U.S. Pat. No. 4,816,567. In some embodiments, isolated nucleic acid encoding an antibody described herein is provided. Such nucleic acid may encode an amino acid sequence comprising the VH of the antibody and/or comprising the VL amino acid sequence (e.g., the light and/or heavy chains of the antibody). In a further embodiment, one or more vectors (e.g., expression vectors) comprising such nucleic acid are provided. In a further embodiment, a host cell comprising such nucleic acid is provided. In some embodiments, a host cell comprises (e.g., has been transformed with): (1) a vector comprising a nucleic acid that encodes an amino acid sequence comprising the VH of the antibody. In some other embodiments, a host cell comprises: (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g., a Chinese Hamster Ovary (CHO) cell or lymphoid cell (e.g., YO, NSO, Sp20 cell). In some embodiments, a method of making an immunoconjugate of the invention is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an immunoconjugate of the present invention, nucleic acid encoding an antibody construct, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and/or light chains of the antibody). Nucleic acid molecules encoding amino acid sequence of the immunoconjugate of the present invention (including sequence variants) may be prepared by a variety of methods known to the skilled worker. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody construct.

### Manipulation of Host Cells for Immunoconjugate Production

Host cells are transfected or transformed with expression or cloning vectors described herein for immunoconjugate of the invention production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Suitable host cells for cloning or expression of immunoconjugate-encoding nucleic acids and vectors include prokaryotic or eukaryotic cells described herein. For example, antibodies may be produced in bacteria, in particular when glycosylation and Fc effector function are not needed. For expression of antibody fragments and polypeptides in bacteria, see e.g., U.S. Pat. Nos. 5,648,237; 5,789,199; 5,840,523; and Charlton, Methods in Molecular Biology, Vol. 248 (B. K. C. Lo, ed., Humana Press, Totowa, NJ, 2003), pp. 245-254, describing expression of antibody fragments in E. coli). After expression, the immunoconjugate may be isolated from the bacterial cell paste in a soluble fraction and can be further purified.

In addition to prokaryotes, eukaryotic microbes such as filamentous fungi or yeast are suitable cloning or expression hosts for immunoconjugate-encoding vectors, including fungi and yeast strains whose glycosylation pathways have been “humanized,” resulting in the production of an antibody with a partially or fully human glycosylation pattern (see e.g., Gerngross, Nat. Biotech. 22:1409-1414 (2004); Li et al., Nat. Biotech. 24:210-215 (2006)).

Suitable host cells for the expression of glycosylated immunoconjugate are also derived from multicellular organisms (e.g., invertebrates and vertebrates). Examples of invertebrate cells include plant and insect cells. Numerous baculoviral strains have been identified which suitable for use in conjunction with insect cells, particularly for transfection of Spodoptera frugiperda cells. Plant cell cultures can also be utilized as hosts (see e.g., U.S. Pat. Nos. 5,959,177; 6,040,498; 6,420,548; 7,125,978; and 6,417,429.

Vertebrate cells may also be used as hosts. For example, mammalian cell lines that are adapted to grow in suspension may be useful. Other examples of useful mammalian host cell lines are monkey kidney CV 1 line transformed by SV40 (COS-7); human embryonic kidney line (293 or 293 cells as described, e.g., in Graham et al., J Gen Viral. 36:59 (1977); baby hamster kidney cells (BHK); mouse Sertoli cells (TM4 cells as described, e.g., in Mather, Biol. Reprod. 23:243-251 (1980); monkey kidney cells (CV 1); African green monkey kidney cells (VERO-76); human cervical carcinoma cells (HELA); canine kidney cells (MOCK; buffalo rat liver cells (BRL 3A); human lung cells (W138); human liver cells (Hep 02); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFK CHO cells (Urlaub et al., Proc Natl Acad Sci USA 77:4216 (1980)); and myeloma cell lines such as YO, NSO and Sp2/0. For a review of certain mammalian host cell lines suitable for immunoconjugate production, see e.g., Yazaki and Wu, Methods in Molecular Biology, Vol. 248 (B. K. C. Lo, ed., Humana Press, Totowa, NJ), pp. 255-268 (2003).

Methods of eukaryotic cell transfection and prokaryotic cell transformation, which means introduction of DNA into the host so that the DNA is replicable, either as an extrachromosomal or by chromosomal integrant, are known to the skilled worker, for example, CaCl2), CaPO4, liposome-mediated, polyethylene-gycol/DMSO and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc Natl Acad Sci USA 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

### Prokaryotic Host Cells

Suitable prokaryotes include but are not limited to archaebacteria and eubacteria, such as Gram-negative or Gram-positive organisms, for example, Enterobacteriaceae such as E. coli. Various E. coli strains are publicly available, such as K12 strain MM294 (ATCC 31,446); X1776 (ATCC 31,537); W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, Rhizobia, Vitreoscilla, Paracoccus and Streptomyces. These examples are illustrative rather than limiting. E. coli strain W3110 is one advantageous host or parent host because it is a common host strain for recombinant DNA product fermentations. Preferably, the host cell secretes minimal amounts of proteolytic enzymes. For example, strain W3110 (Bachmann, Cellular and Molecular Biology, vol. 2 (Washington, D.C.: American Society for Microbiology, 1987), pp. 1190-1219; ATCC Deposit No. 27,325) may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation; E. coli W3110 strain 33D3 having genotype W3110 AfhuA (AtonA) ptr3 lac Iq lacL8 AompTA(nmpc-fepE) degP41 kanR (U.S. Pat. No. 5,639,635) and an E. coli strain having mutant periplasmic protease disclosed in U.S. Pat. No. 4,946,783 issued 7 Aug. 1990. Other strains and derivatives thereof, such as E. coli 294 (ATCC 31,446), E. coli B, E. coli X 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed. Full length antibodies have greater half-life in circulation. Production in E. coli is faster and more cost efficient. For expression of antibody fragments and polypeptides in bacteria, see, e.g., U.S. Pat. Nos. 5,648,237; 5,789,199 and 5,840,523, which describe translation initiation region (TIR) and signal sequences for optimizing expression and secretion. After expression, the immunoconjugate is isolated from the E. coli cell paste in a soluble fraction and can be purified through, e.g., a protein A or G column depending on the isotype. Final purification can be carried out similar to the process for purifying antibody expressed e.g., in CHO cells.

### Eukaryotic Host Cells

In addition to prokaryotes, eukaryotic microbes such as filamentous fungi or yeast are suitable cloning or expression hosts for immunoconjugate of the invention-encoding vectors. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9: 968-75 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc Natl Acad Sci USA 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc Natl Acad Sci USA 81: 1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated immunoconjugate of the invention are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc Natl Acad Sci USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for immunoconjugate of the invention production and cultured in conventional nutrient media modified as appropriate for inducing promoters, selecting transformants, or amplifying the genes encoding the desired sequences.

### Selection and Use of a Replicable Vector

For recombinant production of a radioisotope delivery platform of the invention, the nucleic acid (e.g., cDNA or genomic DNA) encoding it is isolated and inserted into a replicable vector for further cloning (amplification of the DNA) or for expression. DNA encoding the immunoconjugate is readily isolated and sequenced using conventional procedures (e.g., by using oligonucleotide probes that are capable of binding specifically to genes encoding the heavy and light chains of an antibody). Many vectors are available. The choice of vector depends in part on the host cell to be used. Generally, suitable host cells are of either prokaryotic or eukaryotic (generally mammalian) origin.

The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The immunoconjugate of the invention may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the immunoconjugate of the invention-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

### Culturing Host Cells Producing Radioisotope Delivery Platforms

The host cells used to produce the immunoconjugate of the invention of this invention may be cultured in a variety of media and culture conditions.

### Prokaryotic Host Cell Cultures

Prokaryotic cells used to produce the polypeptides of the invention are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In some embodiments of the invention, PhoA promoters are used for controlling transcription of the polypeptides. Accordingly, the transformed host cells are cultured in a phosphate-limiting medium for induction. In some embodiments, the phosphate-limiting medium is the C.R.A.P medium (see, e.g., Simmons et al., J. Immunol. Methods (2002), 263: 133-47). A variety of other inducers may be used, according to the vector construct employed, as is known in the art.

In some embodiments, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In some embodiments of the invention, immunoconjugate production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (a preferred carbon/energy source). Small-scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted immunoconjugate polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274: 19601-5; U.S. Pat. Nos. 6,083,715; 6,027,888; Bothmann and Pluckthun (2000) J. Biol. Chem. 275:17100-5; Ramm and Pluckthun (2000) J. Biol. Chem. 275:17106-13; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; U.S. Pat. Nos. 5,264,365; 5,508,192; Hara et al., Microbial Drug Resistance, 2:63-72 (1996).

In some embodiments, E. coli strains deficient for proteolytic enzymes and transformed with plasmids overexpressing one or more chaperone proteins are used as host cells in the expression system of the invention.

### Eukaryotic Host Cell Cultures

Commercially available media such as Ham's F10 (Sigma), Minimal Essential Medium ((MEM), (Sigma), RPMI-1640 (Sigma), and Dulbecco's Modified Eagle Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Purification of an Immunoglobulin-Derived Structure of the Invention

Forms of immunoconjugate of the invention may be recovered from culture medium or from host cell lysates. If membrane-bound, it can be released from the membrane using a suitable detergent solution (e.g., Triton-X 100) or by enzymatic cleavage. Cells employed in expression of immunoconjugate of the invention can be disrupted by various physical or chemical means, such as freeze-thaw cycling, sonication, mechanical disruption, or cell lysing agents.

It may be desired to purify immunoconjugate of the invention from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the immunoconjugate of the invention. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular immunoconjugate of the invention produced.

When using recombinant techniques, the immunoconjugate can be produced intracellularly, in the periplasmic space, or directly secreted into the medium. If the immunoconjugate is produced intracellularly, as a first step, the particulate debris, either host cells or lysed fragments, are removed, for example, by centrifugation or ultrafiltration. Carter et al., Bio/Technology 10: 163-7 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the immunoconjugate is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The immunoconjugate composition prepared from the cells can be purified using, for example, hydroxylapatite chromatography, gel electrophoresis, dialysis, and affinity chromatography, with affinity chromatography being a preferred purification technique. The suitability of protein A as an affinity ligand depends on the species and isotype of any immunoglobulin Fc domain that is present in the immunoconjugate. Protein A can be used to purify antibodies that are based on human γ1, γ2 or γ4 heavy chains (Lindmark et al., J. Immunol. Meth. 62: 1-13 (1983)). Protein G is recommended for all mouse isotypes and for human 73 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the immunoconjugate comprises a CH3 domain, the Bakerbond ABX™ resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the immunoconjugate to be recovered.

Following any preliminary purification step(s), the mixture comprising the immunoconjugate of interest and contaminants may be subjected to low pH hydrophobic interaction chromatography using an elution buffer at a pH between about 2.5-4.5, and generally at low salt concentrations (e.g., from about 0-0.25M salt).

### Immunoconjugates (Including Antibody Drug Conjugates (ADCs))

In a further embodiment of the invention, an immunoconjugate of the invention according to any of the above embodiments or described herein is conjugated to a heterologous moiety or agent, such as, e.g., as described below and including any additional exogenous material as described herein.

In some embodiments, the invention provides immunoconjugates comprising a multivalent antibody construct of the present invention conjugated to one or more therapeutic agents or radioactive isotopes.

In some embodiments, an immunoconjugate comprises a multivalent antibody construct as described herein conjugated to a radioactive atom to form a radioconjugate. As described herein, a variety of radioactive isotopes are available for the production of radioconjugates of the invention.

Conjugates of an immunconjugate or antibody construct may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate H), active esters (such as disuccinimidyl suberate), aldehydes (such as glutaraldehyde), bis-azido compounds (such as bis (p-azidobenzoyl) hexanediamine), bis-diazonium derivatives (such as bis-(pdiazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an illustrative chelating agent for conjugation of radionucleotide to the antibody (see e.g., WO 1994/11026). The linker may be a “cleavable linker” facilitating release of a cytotoxic drug in the cell. For example, an acid-labile linker, peptidase-sensitive linker, photolabile linker, dimethyl linker or disulfide-containing linker may be used (see e.g., Chari et al., Cancer Res. 52:127-131 (1992); U.S. Pat. No. 5,208,020).

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., obtainable from Pierce Biotechnology, Inc., Rockford, IL., U.S.).

As recognized by the person of ordinary skill in the art, certain methods above are also useful to the preparation of radioimmunoconjugates and targeted imaging complexes (notwithstanding the textual reference to only immunoconjugates or antibody constructs), and such preparative methods are also embraced by the invention.

Immunoconjugate Using Chelators and/or Linkers

Methods for affixing a radioisotope to an immunoconjugate or antibody construct (i.e., “labeling” a multivalent antibody with a radioisotope) are well known to the skilled worker. Certain of these methods are described, for example, in WO 2017/155937.

Bifunctional chelators, such as, e.g., DOTA, DTPA, and related analogs are suitable for coordinating metal ions like α and β-emitting radionuclides. For example, these chelating molecules can be linked to the targeting molecule by forming a new amide bond between an amine on the antibody construct (e.g., a functional group of a lysine residue) and a carboxylate on the DOTA/DTPA. In the case of peptide synthesis, characterization and purification of the linker addition can be part of the overall synthesis of a multivalent antibody platform or immunoconjugate for radioisotope conjugation.

For some embodiment, the method of producing an immunoconjugate involves a click chemistry step described by Poty, S et al., Chem Commun. (Camb) 54: 2599 (2018).

For some embodiments, a peptide may be biosynthesized or may be synthesized by chemical amino acid synthesis using suitable amino acid precursors involving, for example, fluorine-19 in place of hydrogen. In some embodiments, radiolabels may be incorporated into peptide. In some embodiments, radiolabels may be linked to peptide. The IODOGEN method (Fraker et al. (1978) Biochem Biophys Res Commun. 80: 49-57 can be used to incorporate iodine-123. “Monoclonal Antibodies in Immunoscintigraphy” (Chatal, CRC Press 1989) describes other methods in detail.

### Pharmaceutical Compositions

Provided herein are compositions comprising an immunoconjugate or radioimmunoconjugate described herein. The invention further provides pharmaceutical compositions and formulations comprising at least one immunoconjugate of the present invention and at least one pharmaceutically acceptable excipient or carrier. In some embodiments, a pharmaceutical formulation comprises (1) an immunoconjugate or radioimmunoconjugate of the invention, and (2) a pharmaceutically acceptable carrier.

An immunoconjugate or radioimmunoconjugate is formulated in any suitable form for delivery to a target cell/tissue. Pharmaceutical formulations of an immunoconjugate of the present invention are prepared by mixing such immunoconjugate having the desired degree of purity with one or more optional pharmaceutically acceptable carriers, diluents, and/or excipients (Remington's Pharmaceutical Sciences 16th edition, Osol, A. Ed. (1980)), in the form of lyophilized formulations or aqueous solutions. Pharmaceutically acceptable carriers, diluents, and excipients are generally nontoxic to recipients at the dosages and concentrations employed, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutical formulations to be used for in vivo administration are generally sterile. This is readily accomplished by filtration through sterile filtration membranes.

Examples of lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

Pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). In some embodiments, a sHASEGP is combined with one or more additional glycosaminoglycanases such as chondroitinases.

The formulation herein may also contain more than one active ingredient as necessary for the particular indication being treated, preferably those with complementary activities that do not adversely affect each other. Such active ingredients are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

In some embodiments, immunoconjugates may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the immunoconjugate are prepared by methods known in the art, such as described in Epstein et al., Proc Natl Acad Sci USA 82: 3688 (1985); Hwang et al., Proc Natl Acad Sci USA 77: 4030 (1980); U.S. Pat. Nos. 4,485,045 and 4,544,545; and WO1997/38731 published Oct. 23, 1997. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent is optionally contained within the liposome (see Gabizon et al., J. National Cancer Inst. 81: 1484 (1989)). Liposomes with enhanced circulation time are disclosed in U.S. Pat. No. 5,013,556.

Sustained-release preparations may be prepared. Suitable examples of sustained-release preparations include semipermeable matrices of solid hydrophobic polymers containing the antibody, which matrices are in the form of shaped articles, e.g., films, or microcapsules.

### Methods of Use

The immunoconjugates (e.g., radio-immunoconjugates) described herein are useful for treating a disease, disorder, or condition (e.g., a tumor or cancer) in a patient in need thereof, the method comprising administering an immunoconjugate or radioimmunoconjugate or composition described herein. The immunoconjugates are also useful for killing a tumor or cancer cell (e.g., a solid tumor cell expressing FOLR1 or DLL3). Furthermore, the immunoconjugates described herein can be used in methods of labeling or detecting a tumor cell or cancer cell.

In some embodiments, provided herein are methods of killing a tumor cell or a cancer cell, the method comprising: contacting the tumor cell or the cancer cell with a radio-immunoconjugate (e.g., an immunoconjugate comprising a radionuclide) described herein, thereby killing the tumor cell or cancer cell. In some embodiments, the tumor cell is a solid tumor cell. In certain embodiments, the tumor cell cancer and/or cancer cell expresses FOLR1 or DLL3. In certain embodiments, the tumor cell and/or cancer cell is in an individual.

In some embodiments, provided herein are methods of treating a cancer or a tumor in an individual comprising administering to the individual a immunoconjugate (e.g., radio-immunoconjugate) described herein, thereby treating the cancer or the tumor. In certain embodiments, the individual is a human. In certain embodiments, the tumor is a solid tumor and/or the cancer comprises a solid tumor.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, an immunoconjugate or radioimmunoconjugate or composition of the invention can be used in a method for binding target antigen in an individual suffering from a disorder associated with increased target antigen expression and/or activity, the method comprising administering to the individual the immunoconjugate or radioimmunoconjugate or composition such that target antigen in the individual is bound. In some embodiments, the target antigen is human target antigen, and the individual is a human individual. An immunoconjugate or radioimmunoconjugate or composition of the invention can be administered to a human for therapeutic purposes. Moreover, an immunoconjugate or radioimmunoconjugate or composition of the invention can be administered to a non-human mammal expressing target antigen with which the immunoconjugate or radioimmunoconjugate cross-reacts (e.g., a primate, pig, rat, or mouse) for veterinary purposes or as an animal model of human disease. Regarding the latter, such animal models may be useful for evaluating the therapeutic efficacy of an immunoconjugate or radioimmunoconjugate or composition of the invention (e.g., testing of dosages and time courses of administration).

An immunoconjugate or radioimmunoconjugate or composition of the invention (and any additional therapeutic agent or adjuvant) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody (e.g., multivalent antibody) is suitably administered by pulse infusion, particularly with declining doses of the antibody (e.g., multivalent antibody). Dosing can be by any suitable route, e.g., by injections, such as intravenous or subcutaneous injections, depending in part on whether the administration is brief or chronic.

Immunoconjugate or radioimmunoconjugate or compositions of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugates of the invention are administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. For some embodiments, intravenous or subcutaneous administration of the immunoconjugate or radioimmunoconjugate or composition of the invention is preferred.

For the prevention or treatment of disease, the dosage and mode of administration will be chosen by the physician according to known criteria. The appropriate dosage of immunoconjugate or radioimmunoconjugate or composition of the invention will depend on the type of disease to be treated, as defined above, the severity and course of the disease, whether the immunoconjugate or radioimmunoconjugate or composition of the invention is administered for preventive or therapeutic purposes, previous therapy, the patient's clinical history and response to the immunoconjugate or radioimmunoconjugate or composition, and the discretion of the attending physician. The immunoconjugate or radioimmunoconjugate or composition of the invention is suitably administered to the patient at one time or over a series of treatments. Preferably, the immunoconjugate or radioimmunoconjugate or composition is administered by intravenous infusion or by subcutaneous injections. Depending on the type and severity of the disease, about 1 g/kg to about 50 mg/kg body weight (e.g., about 0.1-15 mg/kg/dose) of immunoconjugate or radioimmunoconjugate or composition can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the immunoconjugate or radioimmunoconjugate or composition of the invention. However, other dosage regimens may be useful. A typical daily dosage might range from about 1 g/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

The dose and administration schedule may be selected and adjusted based on the level of disease, or tolerability in the subject, which may be monitored during the course of treatment. The conjugates of the present invention may administered once per day, once per week, multiple times per week, but less than once per day, multiple times per month but less than once per day, multiple times per month but less than once per week, once per month, once per five weeks, once per six weeks, once per seven weeks, once per eight weeks, once per nine weeks, once per ten weeks, or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

For some embodiments, the effective amount of the immunoconjugate or radioimmunoconjugate or composition may be provided as a single dose.

The immunoconjugates and radioimmunoconjugates of the present invention maybe used in combination with conventional and/or novel methods of treatment or therapy or separately as a monotherapy. In some embodiments, the immunoconjugates and radioimmunoconjugates of the present invention maybe used with one or more radiation sensitizer agents. Such agents include any agent that can increase the sensitivity of cancer cells to radiation therapy. In other embodiments, immunoconjugates and radioimmunoconjugates of the present invention may be used in combination with novel and/or conventional agents that can augment the biological effects of radiotherapy. Irradiation of a tumor can cause a variety of biological consequences which can be exploited by combining immunoconjugates and radioimmunoconjugates of the present invention with agents that target relevant pathways. In some embodiments, such agents may reduce tumor angiogenesis, or inhibit local invasion and metastasis, or prevent repopulation, or augment the immune response, or deregulate cellular energetics, or reduce population, or alter tumor metabolism, or increase tumor damage, or reduce DNA repair. In certain embodiments, agents for use in combination with immunoconjugates and radioimmunoconjugates of the present invention may comprise DDR inhibitors, e.g., PARP, ATR, Chk1, or DNA-PK; or survival signaling inhibitors, e.g., mTOR, PI3k, NF-kB; or antihypoxia agents, e.g, HIF-1-alpha, CAP, or UPR; or metabolic inhibitors, e.g., MCT1, MCT4 inhibitors; or immunotherapeutics, e.g., anti-CTLA4, anti-PD-1; or inhibitors of growth factor signal transduction, e.g., EGFR or MAPK inhibitors; or anti-invasives, e.g., kinase inhibitors, chemokine inhibitors, or integrin inhibitors; or anti-angiogenic agents, e.g., VEGF-inhibitors.

Immunoconjugates and radioimmunoconjugates of the present invention may (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind. In this context, “inhibiting cell growth or proliferation” means decreasing a cell's growth or proliferation by at least 10%, 20%, 30%, 40%, 50%, 60%, 70%, 80%, 90%, 95%, or 100%, and includes inducing cell death.

By way of example, an immunoconjugate that inhibits the growth of a tumor cell is one that results in measurable growth inhibition of a tumor cell (e.g., a cancer cell). In some embodiments, an immunoconjugate or radioimmunoconjugate of the invention is capable of inhibiting the growth of cancer cells displaying the antigen bound by the immunoconjugate or radioimmunoconjugate. Preferred growth inhibitory immunoconjugates or radioimmunoconjugates inhibit growth of antigen-expressing tumor cells by greater than 20%, preferably from about 20% to about 50%, and even more preferably, by greater than 50% (e.g., from about 50% to about 100%) as compared to the appropriate control, the control typically being tumor cells not treated with the immunoconjugate or radioimmunoconjugate being tested.

For some embodiments, a majority of the immunoconjugate or radioimmunoconjugate or composition administered to a subject typically consists of non-labeled immunoconjugate, with the minority being labeled radioimmunoconjugate. The ratio of labeled radioimmunoconjugate to non-labeled immunoconjugate can be adjusted using known methods. Thus, accordingly to certain aspects of the present invention, the immunoconjugate or radioimmunoconjugate may be provided in a total protein amount of up to 100 mg, such as less than 60 mg, or from 5 mg to 45 mg, or a total protein amount of between 0.1 μg/kg to 1 mg/kg patient weight, such as 1 μg/kg to 1 mg/kg patient weight, or 10 μg/kg to 1 mg/kg patient weight, or 100 μg/kg to 1 mg/kg patient weight, or 0.1 μg/kg to 100 μg/kg patient weight, or 0.1 μg/kg to 50 μg/kg patient weight, or 0.1 μg/kg to 10 μg/kg patient weight, or 0.1 μg/kg to 40 μg/kg patient weight, or 1 μg/kg to 40 μg/kg patient weight, or 0.1 mg/kg to 1.0 mg/kg patient weight, such as from 0.2 mg/kg patient weight to 0.6 mg/kg patient weight.

In certain embodiments, the immunoconjugate/radioimmunoconjugate may be administered from about 0.5 mg/kg to about 30 mg/kg. In certain embodiments, the immunoconjugate/radioimmunoconjugate may be administered from about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 3 mg/kg, about 0.5 mg/kg to about 4 mg/kg, about 0.5 mg/kg to about 5 mg/kg, about 0.5 mg/kg to about 10 mg/kg, about 0.5 mg/kg to about 20 mg/kg, about 0.5 mg/kg to about 30 mg/kg, about 1 mg/kg to about 2 mg/kg, about 1 mg/kg to about 5 mg/kg, about 1 mg/kg to about 10 mg/kg, about 1 mg/kg to about 3 mg/kg, about 1 mg/kg to about 4 mg/kg, about 1 mg/kg to about 5 mg/kg, about 1 mg/kg to about 10 mg/kg, about 1 mg/kg to about 20 mg/kg, about 1 mg/kg to about 30 mg/kg, about 2 mg/kg to about 5 mg/kg, about 2 mg/kg to about 10 mg/kg, about 2 mg/kg to about 3 mg/kg, about 2 mg/kg to about 4 mg/kg, about 2 mg/kg to about 5 mg/kg, about 2 mg/kg to about 10 mg/kg, about 2 mg/kg to about 20 mg/kg, about 2 mg/kg to about 30 mg/kg, about 5 mg/kg to about 10 mg/kg, about 5 mg/kg to about 3 mg/kg, about 5 mg/kg to about 4 mg/kg, about 5 mg/kg to about 5 mg/kg, about 5 mg/kg to about 10 mg/kg, about 5 mg/kg to about 20 mg/kg, about 5 mg/kg to about 30 mg/kg, about 10 mg/kg to about 3 mg/kg, about 10 mg/kg to about 4 mg/kg, about 10 mg/kg to about 5 mg/kg, about 10 mg/kg to about 10 mg/kg, about 10 mg/kg to about 20 mg/kg, about 10 mg/kg to about 30 mg/kg, about 3 mg/kg to about 4 mg/kg, about 3 mg/kg to about 5 mg/kg, about 3 mg/kg to about 10 mg/kg, about 3 mg/kg to about 20 mg/kg, about 3 mg/kg to about 30 mg/kg, about 4 mg/kg to about 5 mg/kg, about 4 mg/kg to about 10 mg/kg, about 4 mg/kg to about 20 mg/kg, about 4 mg/kg to about 30 mg/kg, about 5 mg/kg to about 10 mg/kg, about 5 mg/kg to about 20 mg/kg, about 5 mg/kg to about 30 mg/kg, about 10 mg/kg to about 20 mg/kg, about 10 mg/kg to about 30 mg/kg, or about 20 mg/kg to about 30 mg/kg. In certain embodiments, the immunoconjugate/radioimmunoconjugate may be administered at about 0.5 mg/kg, about 1 mg/kg, about 2 mg/kg, about 5 mg/kg, about 10 mg/kg, about 3 mg/kg, about 4 mg/kg, about 5 mg/kg, about 10 mg/kg, about 20 mg/kg, or about 30 mg/kg. In certain embodiments, the immunoconjugate/radioimmunoconjugate may be administered at least about 0.5 mg/kg, about 1 mg/kg, about 2 mg/kg, about 5 mg/kg, about 10 mg/kg, about 3 mg/kg, about 4 mg/kg, about 5 mg/kg, about 10 mg/kg, or about 20 mg/kg. In certain embodiments, the immunoconjugate/radioimmunoconjugate may be administered at most about 1 mg/kg, about 2 mg/kg, about 5 mg/kg, about 10 mg/kg, about 3 mg/kg, about 4 mg/kg, about 5 mg/kg, about 10 mg/kg, about 20 mg/kg, or about 30 mg/kg.

In some embodiments, the method comprises administering the effective amount of a radioimmunoconjugate comprising 225-Ac that is from 0.01 to 0.1 mCi, or 0.1 mCi to 1.0 mCi, or from 1.0 mCi to 2.0 mCi, or from 2.0 mCi to 4.0 mCi.

In some embodiments, the method comprises administering the effective amount of a radioimmunoconjugate comprising 225-Ac that is from 0.1 μCi/kg to 2.0 μCi/kg subject weight, or from 0.1 μCi/kg to 1.0 μCi/kg subject weight, or from 1.0 μCi/kg to 3.0 μCi/kg subject weight, or from 3.0 μCi/kg to 10.0 μCi/kg subject weight, or from 10.0 μCi/kg to 20.0 μCi/kg subject weight, or from 10.0 μCi/kg to 30.0 μCi/kg subject weight.

In certain embodiments, the effective amount of 225-Ac is about 0.1 microcurie to about 20 microcurie. In certain embodiments, the effective amount of 225-Ac is about 0.1 microcurie to about 0.2 microcurie, about 0.1 microcurie to about 0.5 microcurie, about 0.1 microcurie to about 1 microcurie, about 0.1 microcurie to about 2 microcurie, about 0.1 microcurie to about 3 microcurie, about 0.1 microcurie to about 4 microcurie, about 0.1 microcurie to about 5 microcurie, about 0.1 microcurie to about 10 microcurie, about 0.1 microcurie to about 20 microcurie, about 0.2 microcurie to about 0.5 microcurie, about 0.2 microcurie to about 1 microcurie, about 0.2 microcurie to about 2 microcurie, about 0.2 microcurie to about 3 microcurie, about 0.2 microcurie to about 4 microcurie, about 0.2 microcurie to about 5 microcurie, about 0.2 microcurie to about 10 microcurie, about 0.2 microcurie to about 20 microcurie, about 0.5 microcurie to about 1 microcurie, about 0.5 microcurie to about 2 microcurie, about 0.5 microcurie to about 3 microcurie, about 0.5 microcurie to about 4 microcurie, about 0.5 microcurie to about 5 microcurie, about 0.5 microcurie to about 10 microcurie, about 0.5 microcurie to about 20 microcurie, about 1 microcurie to about 2 microcurie, about 1 microcurie to about 3 microcurie, about 1 microcurie to about 4 microcurie, about 1 microcurie to about 5 microcurie, about 1 microcurie to about 10 microcurie, about 1 microcurie to about 20 microcurie, about 2 microcurie to about 3 microcurie, about 2 microcurie to about 4 microcurie, about 2 microcurie to about 5 microcurie, about 2 microcurie to about 10 microcurie, about 2 microcurie to about 20 microcurie, about 3 microcurie to about 4 microcurie, about 3 microcurie to about 5 microcurie, about 3 microcurie to about 10 microcurie, about 3 microcurie to about 20 microcurie, about 4 microcurie to about 5 microcurie, about 4 microcurie to about 10 microcurie, about 4 microcurie to about 20 microcurie, about 5 microcurie to about 10 microcurie, about 5 microcurie to about 20 microcurie, or about 10 microcurie to about 20 microcurie. In certain embodiments, the effective amount of 225-Ac is about 0.1 microcurie, about 0.2 microcurie, about 0.5 microcurie, about 1 microcurie, about 2 microcurie, about 3 microcurie, about 4 microcurie, about 5 microcurie, about 10 microcurie, or about 20 microcurie. In certain embodiments, the effective amount of 225-Ac is at least about 0.1 microcurie, about 0.2 microcurie, about 0.5 microcurie, about 1 microcurie, about 2 microcurie, about 3 microcurie, about 4 microcurie, about 5 microcurie, or about 10 microcurie. In certain embodiments, the effective amount of 225-Ac is at most about 0.2 microcurie, about 0.5 microcurie, about 1 microcurie, about 2 microcurie, about 3 microcurie, about 4 microcurie, about 5 microcurie, about 10 microcurie, or about 20 microcurie. According to embodiments where the radioisotope of the radioimmunoconjugate is 111-In, the effective amount is below, for example, 15.0 mCi (i.e., where the amount of 111-In administered to the subject delivers a total body radiation dose of below 15.0 mCi).

According to embodiments where the radioisotope of the radioimmunoconjugate is 111-In, the effective amount is below 15.0 mCi, below 14.0 mCi, below 13.0 mCi, below 12.0 mCi, below 11.0 mCi, below 10.0 mCi., below 9.0 mCi, below 8.0 mCi, below 7.0 mCi, below 6.0 mCi, below 5.0 mCi, below 4.0 mCi, below 3.5 mCi, below 3.0 mCi, below 2.5 mCi, below 2.0 mCi, below 1.5 mCi, below 1.0 mCi, below 0.5 mCi, below 0.4 mCi, below 0.3 mCi, below 0.2 mCi, or below 0.1 mCi.

According to embodiments where the radioisotope of the radioimmunoconjugate is 111-In, the effective amount is from 0.1 mCi to 1.0 mCi, from 0.1 mCi to 2.0 mCi, from 1.0 mCi to 2.0 mCi, from 1.0 mCi to 3.0 mCi, from 1.0 mCi to 4.0 mCi, from 1.0 mCi to 5.0 mCi, from 1.0 mCi to 10.0 mCi, from 1.0 mCi to 15.0 mCi, from 1.0 mCi to 20.0 mCi, from 2.0 mCi to 3.0 mCi, from 3.0 mCi to 4.0 mCi, from 4.0 mCi to 5.0 mCi, from 5.0 mCi to 10.0 mCi, from 5.0 mCi to 15.0 mCi, from 5.0 mCi to 20.0 mCi, from 6.0 mCi to 14.0 mCi, from 7.0 mCi to 13.0 mCi, from 8.0 mCi to 12.0 mCi, from 9.0 mCi to 11.0 mCi, or from 10.0 mCi to 15.0 mCi.

According to embodiments where the radioisotope of the radioimmunoconjugate is 111-In, the effective amount is 15.0 mCi, 14.0 mCi, 13.0 mCi, 12.0 mCi, 11.0 mCi, 10.0 mCi, 9.0 mCi, 8.0 mCi, 7.0 mCi, 6.0 mCi, 5.0 mCi, 4.0 mCi, 3.5 mCi, 3.0 mCi, 2.5 mCi, 2.0 mCi, 1.5 mCi, 1.0 mCi, 0.5 mCi, 0.4 mCi, 0.3 mCi, 0.2 mCi, or 0.1 mCi.

According to embodiments where the radioisotope of the radioimmunoconjugate is 225-Ac, the effective amount is below, for example, 30.0 μCi/kg (i.e., where the amount of 225-Ac administered to the subject delivers a radiation dose of below 30.0 μCi per kilogram of subject's body weight).

According to embodiments where the radioisotope of the radioimmunoconjugate is 225-Ac, the effective amount is below 30 μCi/kg, 25 μCi/kg, 20 μCi/kg, 17.5 μCi/kg, 15.0 μCi/kg, 12.5 μCi/kg, 10.0 μCi/kg, 9 μCi/kg, 8 μCi/kg, 7 μCi/kg, 6 μCi/kg, 5 μCi/kg, 4.5 μCi/kg, 4.0 μCi/kg, 3.5 μCi/kg, 3.0 μCi/kg, 2.5 μCi/kg, 2.0 μCi/kg, 1.5 μCi/kg, 1.0 μCi/kg, 0.9 μCi/kg, 0.8 μCi/kg, 0.7 μCi/kg, 0.6 μCi/kg, 0.5 μCi/kg, 0.4 μCi/kg, 0.3 μCi/kg, 0.2 μCi/kg, 0.1 μCi/kg, or 0.05 μCi/kg.

According to embodiments where the radioisotope of the radioimmunoconjugate is 225-Ac, the effective amount is from 0.05 μCi/kg to 0 0.1 μCi/kg, from 0 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8 μCi/kg, from 0.8 μCi/kg to 0.9 μCi/kg, from 0.9 μCi/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg.

According to embodiments where the radioisotope of the radioimmunoconjugate is 225-Ac, the effective amount is 0.05 μCi/kg, 0.1 μCi/kg, 0.2 μCi/kg, 0.3 μCi/kg, 0.4 μCi/kg, 0.5 μCi/kg, 0.6 μCi/kg, 0.7 μCi/kg, 0.8 μCi/kg, 0.9 μCi/kg, 1.0 μCi/kg, 1.5 μCi/kg, 2.0 μCi/kg, 2.5 μCi/kg, 3.0 μCi/kg, 3.5 μCi/kg, 4.0 μCi/kg or 4.5 μCi/kg, 5.0 μCi/kg, 6.0 μCi/kg, 7.0 μCi/kg, 8.0 μCi/kg, 9.0 μCi/kg, 10.0 μCi/kg, 12.5 μCi/kg, 15.0 μCi/kg, 17.5 μCi/kg, 20.0 μCi/kg, 25 μCi/kg, or 30 μCi/kg.

In certain embodiments where the radioisotope of the radioimmunoconjugate is 177-Lu the effective amount is from 0.1 uCi to 100 mCi per meter squared of body surface area.

In certain embodiments where the radioisotope of the radioimmunoconjugate is 177-Lu the effective amount is from 1 mCi to 100 mCi per meter squared of body surface area. In certain embodiments, the effective amount is about 1 per meter squared to about 100 per meter squared. In certain embodiments, the effective amount is about 1 per meter squared to about 5 per meter squared, about 1 per meter squared to about 10 per meter squared, about 1 per meter squared to about 15 per meter squared, about 1 per meter squared to about 20 per meter squared, about 1 per meter squared to about 25 per meter squared, about 1 per meter squared to about 75 per meter squared, about 1 per meter squared to about 100 per meter squared, about 5 per meter squared to about 10 per meter squared, about 5 per meter squared to about 15 per meter squared, about 5 per meter squared to about 20 per meter squared, about 5 per meter squared to about 25 per meter squared, about 5 per meter squared to about 75 per meter squared, about 5 per meter squared to about 100 per meter squared, about 10 per meter squared to about 15 per meter squared, about 10 per meter squared to about 20 per meter squared, about 10 per meter squared to about 25 per meter squared, about 10 per meter squared to about 75 per meter squared, about 10 per meter squared to about 100 per meter squared, about 15 per meter squared to about 20 per meter squared, about 15 per meter squared to about 25 per meter squared, about 15 per meter squared to about 75 per meter squared, about 15 per meter squared to about 100 per meter squared, about 20 per meter squared to about 25 per meter squared, about 20 per meter squared to about 75 per meter squared, about 20 per meter squared to about 100 per meter squared, about 25 per meter squared to about 75 per meter squared, about 25 per meter squared to about 100 per meter squared, or about 75 per meter squared to about 100 per meter squared. In certain embodiments, the effective amount is about 1 per meter squared, about 5 per meter squared, about 10 per meter squared, about 15 per meter squared, about 20 per meter squared, about 25 per meter squared, about 75 per meter squared, or about 100 per meter squared. In certain embodiments, the effective amount is at least about 1 per meter squared, about 5 per meter squared, about 10 per meter squared, about 15 per meter squared, about 20 per meter squared, about 25 per meter squared, or about 75 per meter squared. In certain embodiments, the effective amount is at most about 5 per meter squared, about 10 per meter squared, about 15 per meter squared, about 20 per meter squared, about 25 per meter squared, about 75 per meter squared, or about 100 per meter squared.

According to certain embodiments of the present invention, a preparation of radioimmunoconjugate of the invention, or a composition thereof (e.g., a pharmaceutical composition), may comprise a radiolabeled fraction (radioimmunoconjugate) and an unlabeled fraction (immunoconjugate), wherein the ratio of labeled:unlabeled may be from about 1:1000 to 1:1.

Moreover, the pharmaceutical compositions may be provided as a single dose composition tailored to a specific patient, i.e., as a patient specific therapeutic composition, wherein the amount of labeled and unlabeled immunoconjugate (labeled immunoconjugate, for clarity, being the same as radioimmunoconjugate herein) in the composition may depend on at least a patient weight, height, body surface area, age, gender, and/or disease state or health status. As such, a total volume of the patient specific therapeutic composition may be provided in a vial that is configured to be wholly administered to the patient in one treatment session, such that little to no composition remains in the vial after administration.

Currently, depending on the stage of the cancer, cancer treatment involves one or a combination of the following therapies: surgery to remove the cancerous tissue, radiation therapy, and chemotherapy. Therapy using radioimmunoconjugate of the invention (interchangeably, “radiolabeled immunoconjugate”) may be especially desirable in elderly patients who do not tolerate the toxicity and side effects of chemotherapy well and in metastatic disease where radiation therapy has limited usefulness. For some embodiments, therapy using radiolabeled immunoconjugate of the invention are useful to alleviate target antigen-expressing cancers upon initial diagnosis of the disease or during relapse.

In some embodiments, determining whether a cancer is amenable to treatment by methods disclosed herein involves detecting the presence of the target antigen in a subject or in a sample from a subject. To determine target antigen expression in a cancer, various detection assays are available. In some embodiments, target antigen overexpression is analyzed by immunohistochemistry (IHC). Parrafin embedded tissue sections from a tumor biopsy are subjected to the IHC assay and accorded a target antigen staining intensity criteria. Alternatively, or additionally, FISH assays such as the INFORM® (sold by Ventana, AZ, U.S.A.) or PATHVISION® (Vysis, IL, U.S.A.) may be carried out on formalin-fixed, paraffin-embedded tumor tissue to determine the extent (if any) of target antigen overexpression in the tumor.

Target antigen overexpression or amplification may be evaluated using an in vivo detection assay, e.g., by administering a molecule (such as an antibody construct or immunoconjugate of the invention) which binds the molecule to be detected and is tagged with a detectable label (e.g., a radioactive isotope or a fluorescent label) and externally scanning the patient for localization of the label.

### 2. Using Immunoconjugates and Radioimmunoconjugates of the Invention for Killing a Cell(s)

An immunoconjugate or radioimmunoconjugate of the invention may be used in, for example, in vitro, ex vivo, and in vivo methods. In some embodiments, the invention provides methods for inhibiting cell growth or proliferation, either in vivo or in vitro, the method comprising exposing a cell to an immunoconjugate or radioimmunoconjugate of the invention under conditions permissive for binding of the immunoconjugate or radioimmunoconjugate to a target antigen. The immunoconjugate or radioimmunoconjugate of the invention may also (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

In some embodiments, the invention provides a method of killing an antigen expressing cell, the method comprising contacting the cell with an immunoconjugate or radioimmunoconjugate of the present invention (or a composition thereof). This method can be used, e.g., to kill, deplete, or eliminate target antigen-expressing cells from a population of mixed cells. This method can be used, e.g., to kill, deplete, or eliminate target antigen-expressing cells from a population of mixed cells as a step in the purification of other cells. This method can be performed in vitro or in vivo, including ex vivo on primary patient cell or tissue compositions to prepare such compositions for transplantation.

In some embodiments, an immunoconjugate or radioimmunoconjugate of the invention is used to treat or prevent a cell proliferative disorder. In certain embodiments, the cell proliferative disorder comprises a solid tumor cancer. A solid tumor cancer is a cancer comprising an abnormal mass of tissue, e.g., carcinomas and sarcomas. In certain other embodiments, the cell proliferative disorder comprises a liquid tumor cancer or hematological cancer, Used interchangeably, such cancers present in the body fluid, e.g., leukemias and lymphomas. In certain embodiments, the cell proliferative disorder is associated with increased expression and/or activity of a target antigen. For example, in certain embodiments, the cell proliferative disorder is associated with increased expression of target antigen on the surface of a cell. In certain embodiments, the cell proliferative disorder is a tumor or a cancer. In certain embodiments, the cell proliferative disorder comprises a solid tumor cancer. A solid tumor cancer is a cancer comprising an abnormal mass of tissue, e.g., carcinomas and sarcomas. In certain other embodiments, the cell proliferative disorder comprises a liquid tumor cancer or hematological cancer, Used interchangeably, such cancers present in the body fluid, e.g., leukemias and lymphomas.

In some embodiments, the invention provides methods for treating a cell proliferative disorder comprising administering to an individual an effective amount of an immunoconjugate or radioimmunoconjugate of the invention.

In addition to direct cell killing of target cells expressing cell-surface antigen specifically bound by the immunoconjugate or radioimmunoconjugate of the invention, the immunoconjugate or radioimmunoconjugate of the present invention optionally may be used for delivery of additional cargos to the vicinity of or the interiors of target cells. The delivery of additional exogenous materials may be used, e.g., for cytotoxic, cytostatic, information gathering, and/or diagnostic functions. Non-cytotoxic variants of the immunoconjugate or radioimmunoconjugate of the invention, or optionally toxic variants, may be used to deliver cargos to and/or label the interiors of cells expressing the target antigen. Non-limiting examples of cargos include cytotoxic agents, detection-promoting agents, and small molecule chemotherapeutic agents.

As described herein, in some embodiments, the antibody (e.g., multivalent antibody) constructs, immunoconjugates, radioimmunoconjugates and targeted imaging complexes of the present invention have various non-therapeutic applications. In some embodiments, the compositions of the invention may be used to identify patient populations predicted to benefit from a specific therapeutic approach or modality, such as, e.g., treatment with an immunoconjugates or radioimmunoconjugates of the invention. In some embodiments, the compositions of the invention can be useful for staging of target antigen expressing cancers (e.g., by radioimaging) or as prognostic indicators of disease progression. In some embodiments, the compositions are also useful for detection and quantitation of a target epitope in vitro, e.g., in an ELISA or a Western blot, as well as purification or immunoprecipitation of a target antigen from cells or a tissue sample.

For some embodiments, the immunoconjugate or radioimmunoconjugate of the invention is used in a method to detect the presence of or level of an antigen, such as, e.g., in vitro in a biological sample or in vivo using an imagine technique. Immunoconjugate and radioimmunoconjugate detection can be achieved via different techniques known to the skilled worker and as described herein, e.g., IHC and PET imaging. When an immunoconjugate or radiolabeled immunoconjugate of the invention is used for detection, it may comprise a radioactive atom for scintigraphic studies, for example 99m-Tc or 111-In.

Labelled immunoconjugates of the invention are useful as imaging biomarkers and probes by the various methods and techniques of biomedical and molecular imaging such as: (i) MRI (magnetic resonance imaging); (ii) MicroCT (computerized tomography); (iii) SPECT (single photon emission computed tomography); (iv) PET (positron emission tomography) Chen et al Bioconjugate Chem. 15: 41-9 (2004); (v) bioluminescence; (vi) fluorescence; and (vii) ultrasound. Immunoscintigraphy is an imaging procedure in which antibodies labeled with radioactive substances are administered to an animal or human patient and a picture is taken of sites in the body where the antibody (e.g., multivalent antibody) localizes (U.S. Pat. No. 6,528,624). Imaging biomarkers may be objectively measured and evaluated as an indicator of normal biological processes, pathogenic processes, or pharmacological responses to a therapeutic intervention.

Another embodiment of the present invention is a method of determining the presence of a target antigen in a sample suspected of containing the target antigen, wherein the method comprises exposing the sample to an immunoconjugate that binds to the target antigen and determining binding of the immunconjugate to the target antigen in the sample, wherein the presence of such binding is indicative of the presence of the target antigen in the sample. Optionally, the sample may contain cells (which may be cancer cells) suspected of expressing the target antigen. The immunoconjugate employed in the method may optionally be detectably labeled, attached to a solid support, or the like.

Another embodiment of the present invention is directed to a method of diagnosing the presence of a tumor in a subject, wherein the method comprises (a) contacting a test sample comprising tissue cells obtained from the mammal with an immunoconjugate that binds to a target antigen and (b) detecting the formation of a complex between the immunoconjugate and the target antigen in the test sample, wherein the formation of a complex is indicative of the presence of a tumor in the mammal. Optionally, the immunoconjugate is detectably labeled, attached to a solid support, or the like, and/or the test sample of tissue cells is obtained from an individual suspected of having a cancerous tumor.

In some embodiments, the immunoconjugates of the present invention, including compositions comprising the aforementioned and/or provided herein are useful for detecting the presence of a target antigen, e.g., in vivo or in a biological sample. The immunoconjugates of the invention can be used in a variety of different assays, including but not limited to ELISA, bead-based immunoassays, and mass spectrometry.

In some embodiments, the immunoconjugates of the present invention are useful to quantitate target antigen amounts in a sample. In some embodiments, a biological sample is a biological fluid, such as whole blood or whole blood components including red blood cells, white blood cells, platelets, serum and plasma, ascites, vitreous fluid, lymph fluid, synovial fluid, follicular fluid, seminal fluid, amniotic fluid, milk, saliva, sputum, tears, perspiration, mucus, cerebrospinal fluid, urine and other constituents of the body that may contain the target antigen of interest. In various embodiments, the sample is a body sample from any animal. In some embodiments, the sample is from a mammal. In some embodiments, the sample is from a human subject. In some embodiments, the biological sample is serum from a clinical patient. In some embodiments, the biological sample is biopsy material. In some embodiments, the biological sample is biopsy material from a clinical patient. In some embodiments, the biological sample is serum from a clinical patient. In some embodiments, the biological sample is primary cell culture material. In some embodiments, the biological sample is primary cell culture material from a clinical patient. In some embodiments, the biological sample is from clinical patients or patients treated with a therapeutic antibody or antibodies that binds the same target antigen.

In some embodiments, the sample is from a mammal. In some embodiments, the sample is from a human subject, e.g., when measuring antigen expression in a clinical sample. In some embodiments, the biological sample is from clinical patients or a patient treated with a therapy/therapeutic (e.g., an antibody therapy targeting the same target antigen). In some embodiments, the biological sample is serum or plasma. In some embodiments, the biological sample is serum from a clinical patient. In some embodiments, the biological sample is biopsy material. In some embodiments, the biological sample is biopsy material from a clinical patient. In some embodiments, the biological sample is serum from a clinical patient. In some embodiments, the biological sample is primary cell culture material. In some embodiments, the biological sample is primary cell culture material from a clinical patient.

In some embodiments, compositions comprising ‘labeled’ immunoconjugates are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, fluorophores such as rare earth chelates or fluorescein and its derivatives, rhodamine and its derivatives, dansyl, umbelliferone, luciferases, e.g., firefly luciferase and bacterial luciferase, luciferin, 2,3-dihydrophthalazinediones, horseradish peroxidase (HRP), alkaline phosphatase, J3-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the immunoconjugates or antibody constructs of the invention with the above-described fluorescent, chemiluminescent, and enzyme labels (see e.g., U.S. Pat. No. 3,645,090 (enzymes); U.S. Pat. No. 3,940,475 (fluorimetry), Hunter et al., Nature, 144:945 (1962); David et al., Biochemistry, 13:1014-1021 (1974); Pain et al., J. Immunol. Methods, 40:219-230 (1981); Nygren, J. Histochem and Cytochem, 30:407-412 (1982).

The conjugation of such label, including the enzymes, to the immunconjugate or antibody construct is a standard manipulative procedure for one of ordinary skill in immunoassay techniques (see e.g., O'Sullivan et al. “Methods for the Preparation of Enzyme-antibody Conjugates for Use in Enzyme Immunoassay,” in Methods in Enzymology, ed. J. Langone and H. Van Vunakis, Vol. 73 (Academic Press, New York, New York, 1981), pp. 147-166). Suitable commercially available labeled antibodies may also be used.

Following the addition of last labeled immunoconjugate, the amount of bound immunoconjugate is determined by removing excess unbound labeled immunoconjugate through washing and then measuring the amount of the attached label using a detection method appropriate to the label and correlating the measured amount with the amount of the immunoconjugate of interest in the biological sample. For example, in the case of enzymes, the amount of color developed and measured will be a direct measurement of the amount of the immunoconjugate of interest present. Specifically, if HRP is the label, the color may be detected using the substrate TMD, using a 450 nm read wavelength and a 620 or 630 nm reference wavelength.

In one example, after an enzyme-labeled second antibody directed against the unlabeled immunoconjugate is washed from the immobilized phase, color or chemiluminescence is developed and measured by incubating the immobilized capture reagent with a substrate of the enzyme. Then the concentration of the antibody (e.g., multivalent antibody) of interest is calculated by comparing with the color or chemiluminescence generated by the immunoconjugate of interest run in parallel.

In some embodiments, the method involves a bead-based immunoassay, an ELISA assay, or a mass spectrometric technique. The mass analyzers of such mass spectrometers include, but are not limited to, quadrupole (Q), time of flight (TOF), ion trap, magnetic sector or Fourier transform ion cyclotron resonance (FT-ICR) or combinations thereof. The ion source of the mass spectrometer should yield mainly sample molecular ions, or pseudo-molecular ions, and certain characterizable fragment ions. Examples of such ion sources include atmospheric pressure ionization sources, e.g., electrospray ionization (ESI) and atmospheric pressure chemical ionization (APCI) and Matrix Assisted Laser Desorption Ionization (MALDI). ESI and MALDI are the two most commonly employed methods to ionize proteins for mass spectrometric analysis of small molecules, such as, e.g., by liquid chromatography mass spectrometry (LC/MS) (Lee, M., LC/MS Applications in Drug Development (2002) J. Wiley & Sons, New York). Another example is surface enhanced laser desorption ionization (SELDI). SELDI is a surface-based ionization technique that allows for high-throughput mass spectrometry. Typically, SELDI is used to analyze complex mixtures of proteins and other biomolecules. SELDI employs a chemically reactive surface such as a “protein chip” to interact with analytes, e.g., proteins, in solution. Such surfaces selectively interact with analytes and immobilize them thereon. Thus, the analytes of the invention can be partially purified on the chip and then quickly analyzed in the mass spectrometer. By providing multiple reactive moieties at different sites on a substrate surface, throughput may be increased.

In another embodiment, the invention provides a method for detecting in a biological sample an antigen, the method comprising: (a) contacting the biological sample with an immunoconjugate described herein to allow forming an immunocomplex; (b) detecting or measuring the level of the immunoconjugate bound to the sample. In some embodiments, the immunoconjugate is immobilized to a solid support. In some embodiments, the immobilized immunoconjugate is conjugated to biotin and bound to a streptavidin coated microtiter plate.

### EXAMPLES

The following examples are included for illustrative purposes only and are not intended to limit the scope of the present disclosure.

### Example 1. VHH-Fc Preparation

VHH-Fc plasmids were generated by cloning the VHH sequence, with a hinge and Fc portion(human IgG1 CH2-CH3) into a mammalian expression vector. In some instances, mutations were introduced into the Fc portion. To produce recombinant VHH-Fc and variants thereof, plasmid was transfected into HEK293.SUS cells (ATUM, or similar). After 3-5 days of secretion, the antibody-containing supernatant was cleared of cells by centrifugation and sterile filtration. Antibodies were purified using Mab Select SuRe PCC column (GE, Cat #: 11003495) and buffer exchange into PBS, pH 7.0. Proteins were quantified using A280 or BCA. The purity of the antibodies were tested by SDS-PAGE, capillary electrophoresis, HPLC-SEC and LC-MS using standard protocols. Regarding VHH polypeptides, see, for example, McMahon et al., Nature Structural & Molecular Biology|VOL 25|MARCH 2018|289-296 Yeast surface display platform for rapid discovery of conformationally selective nanobodies; Moutel et al., eLife 2016; 5:e16228 NaLi-H1: A universal synthetic library of humanized nanobodies providing highly functional antibodies and intrabodies. De Genst E, Saerens D, Muyldermans S, Conrath K. Antibody repertoire development in camelids. Dev Comp Immunol. 2006; 30(1-2):187-98. doi: 10.1016/j.dci.2005.06.010. PMID: 16051357. Vincke C, Gutierrez C, Wernery U, Devoogdt N, Hassanzadeh-Ghassabeh G, Muyldermans S. Generation of single domain antibody fragments derived from camelids and generation of manifold constructs. Methods Mol Biol. 2012; 907:145-76. doi: 10.1007/978-1-61779-974-7_8. PMID: 22907350. Arbabi Ghahroudi M, Desmyter A, Wyns L, Hamers R, Muyldermans S. Selection and identification of single domain antibody fragments from camel heavy-chain antibodies. FEBS Lett. 1997 Sep. 15; 414(3):521-6. doi: 10.1016/s0014-5793(97)01062-4. PMID: 9323027.

For VHH humanization, see, for example, Vincke C, Loris R, Saerens D, Martinez-Rodriguez S, Muyldermans S, Conrath K. General strategy to humanize a camelid single-domain antibody and identification of a universal humanized nanobody scaffold. J Biol Chem. 2009 Jan. 30; 284(5):3273-84. doi: 10.1074/jbc.M806889200. Epub 2008 Nov. 14. PMID: 19010777.

For VHH stability, see, for example, Kunz P, Flock T, Soler N, Zaiss M, Vincke C, Sterckx Y, Kastelic D, Muyldermans S, Hoheisel JD. Exploiting sequence and stability information for directing nanobody stability engineering. Biochim Biophys Acta Gen Subj. 2017 September; 1861(9):2196-2205. doi: 10.1016/j.bbagen.2017.06.014. Epub 2017 June 20. PMID: 28642127; PMCID: PMC5548252; Kunz P, Zinner K, Mucke N, Bartoschik T, Muyldermans S, Hoheisel JD. The structural basis of nanobody unfolding reversibility and thermoresistance. Sci Rep. 2018 May 21; 8(1):7934. doi: 10.1038/s41598-018-26338-z. PMID: 29784954; PMCID: PMC5962586.

A number of VHH-Fc prototypes and variants were engineered using VHH sequences such as the anti-HER2 clone 2RS15d VHH (See. e.g., WO2016/016021) (SEQ ID NO: 20), and the anti-DLL3 clone hzIOD9v7.251 VHH sequences (See e.g., W02020/07967) (SEQ ID NO: 30), unless otherwise stated herein the data collected and shown was obtained using VHH antigen binding regions of these clones.

TABLE 1
Constructs

| | | Fc | |
|---|---|---|---|
| VHH Fc | FcRn | Effector | |
| name | Mutant | Mutant | Target |
| H101 | wt | wt | HER2 |
| D102 | wt | wt | DLL3 |
| H105 | I253A | wt | HER2 |
| H106 | S254A | wt | HER2 |
| H107 | H310A | wt | HER2 |
| H108 | H435Q | wt | HER2 |
| H109 | Y463A | wt | HER2 |
| D111 | I253A | wt | DLL3 |
| D112 | S254A | wt | DLL3 |
| D113 | H310A | wt | DLL3 |
| D114 | H435Q | wt | DLL3 |
| D115 | Y463A | wt | DLL3 |
| H133 | wt | AEASS | HER2 |
| D134 | wt | AEASS | DLL3 |
| H135 | H310A | AEASS | HER2 |
| D136 | H310A | AEASS | DLL3 |
| H137 | H435Q | AEASS | HER2 |
| D138 | H435Q | AEASS | DLL3 |

Variants per EU numbering; AEASS = L234A, L235E, G237A, A330S, and P331S

### Example 2. Antibody Binding Properties: Assays for Target Protein and Target Cells

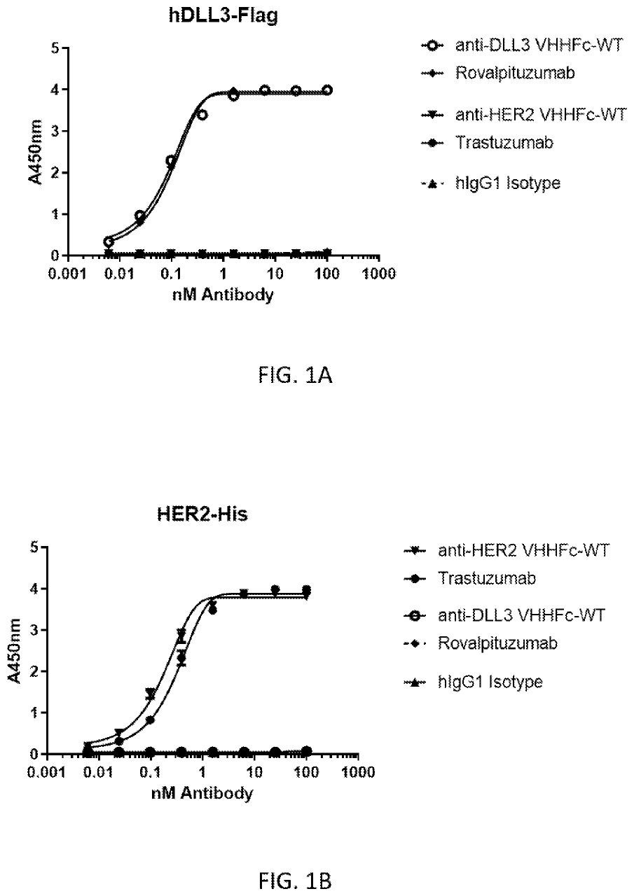

The VHH-Fcs were assessed by ELISA for binding to Target soluble protein—human, murine and cynomolgous orthologs as appropriate, according to standard protocols. Antigens were sourced commercially or produced by cloning known antigen sequences (Uniprot) into mammalian expression vectors with a HIS, FLAG or equivalent tag for purification and detection purposes. A commercially available control anti-target IgG was included. Plates (96-well maxisorp, Corning 3368) were coated with 50 to 100 μL of each Target protein of interest at a concentration optimized for coating. Purified VHH-Fc andhIgG1 isotype control (Sigma, Cat #I5154) were prepared at starting concentrations of 200 to 400 nM and titrated 1:4 down. Following primary antibody incubation for 1 hour at room temperature (RT), and washing, 0.2 ug/ml of secondary HRP-labelled antibody was added and incubated for 1 h at RT (goat anti human-IgG-Fc-HRP Jackson, Cat #109-035-098). Reaction was detected using 50 L/well of TMB (Neogen, Cat #308177). The color development was stopped with 1 M HCl (50 l). Optical density (OD) was measured at 450 nm using Spectromax plate reader and data were processed using SoftMaxPro. Data shows anti-Target VHH-Fcs bind to human, murine and cynomolgous target protein. Recombinant DLL3 protein used was human DLL3.FLAG(Adipogen #AG-40B-0151, amino acid 27-466), or human DLL3.HIS (abcam #ab255797, amino acid 27-492), or murine DLL3.HIS (IPA custom, amino acid 25-477) or cynomolgous DLL3.HIS (Acrobiosystems #, amino acid 27-490). Control antibodies for DLL3 binding was Rovalpituzumab (Creative Biolabs #TAB-216CL) Recombinant HER2 protein used was human Her2.HIS (Sinobiologics, #10004-H08H) and murine HER2.HIS (Sinobiologics #50714-M08H). Control antibody for HER2 binding was Trastuzumab (DIN: 02240692, ROCHE).). FIGS. 1A and 1B show Anti-Her2 and anti-DLL3 VHH-Fcs binding specifically to soluble target antigen in an ELISA, additional VHH-Fcs comprising mutations in the Fc region to decrease effector function and/or FcRn binding were tested but did not significantly affect binding to target antigen.

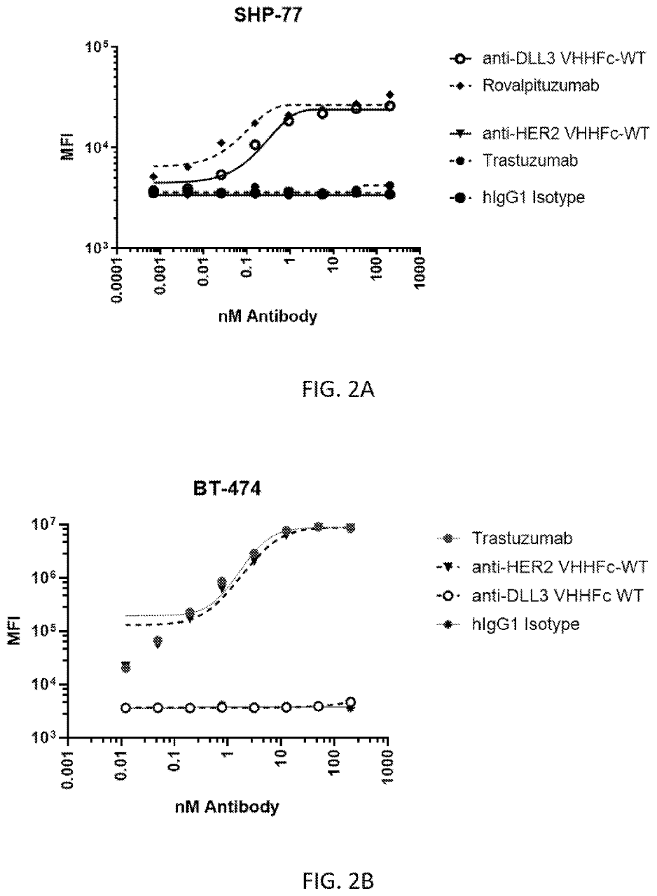

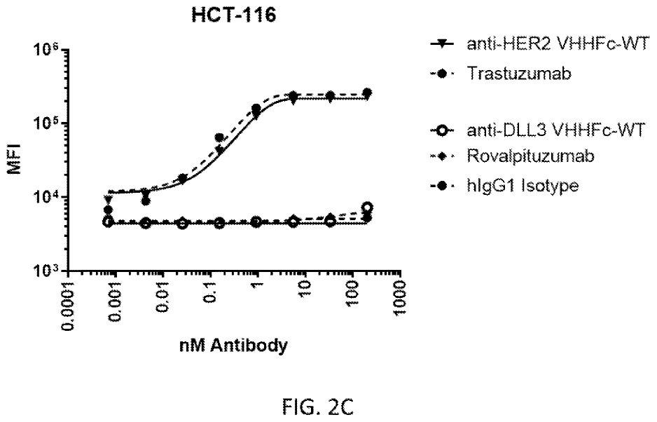

VHH-Fcs were screened for binding to a range of target-positive cancer cell lines by flow cytometry. All cell lines were sourced from ATCC unless otherwise noted, and cultured according to manufacturers instructions and recommended media. HER2-positive cell lines used were SKBR3(ATCC #HTB-30) and BT474(ATCC #HTB-20) and HEK293-6E(NRC) cells. DLL3-positive cell lines tested include SHP-77(ATCC CR1-2195), NCI-H82(ATCC HTB-175), NCI-H69(ATCC HTB-119), HEK-DLL3 (Creative Biogene #CSC-R00531). HER2-negative cell lines tested included SHP-77. DLL3-negative cell lines tested included HCT-116 (CCL-247), BT-474 and SKBR3. Primary antibodies diluted in same manner as for ELISA were added to cells and incubated for 1 hour on ice. Cells were washed twice with 1% FBS in PBS, centrifuged at 450G for 4 minutes and incubated with 2 μg/mL AlexaFluor 647 conjugated anti-human IgG (Jackson, Cat #109-605-098) or AlexaFluor 647 conjugated anti-mouse IgG (Jackson, Cat #115-605-164) with 1:1000 DAPI (Biolegend, Cat #422801) for 30 minutes on ice. Following two further washes, cells were resuspended, and analyzed by flow cytometry on the iQue screener platform (Intellicyt), and data was processed with Forecyt, according to standard protocols. FIGS. 2A, 2B and 2C show binding to target-positive cell lines and shows that binding was specific to Target-positive cells (i.e., through binding comparison to negative controls cells). Further experiments indicated that Fc mutations to reduce effector function and/or FcRn binding did not impact binding to cancer cells as compared to wildtype Fcs.

### Example 3. Internalization Assays

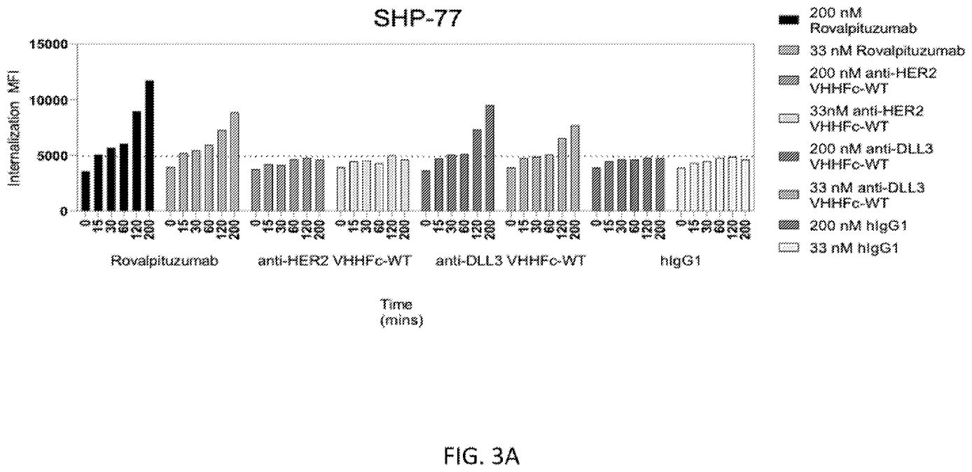

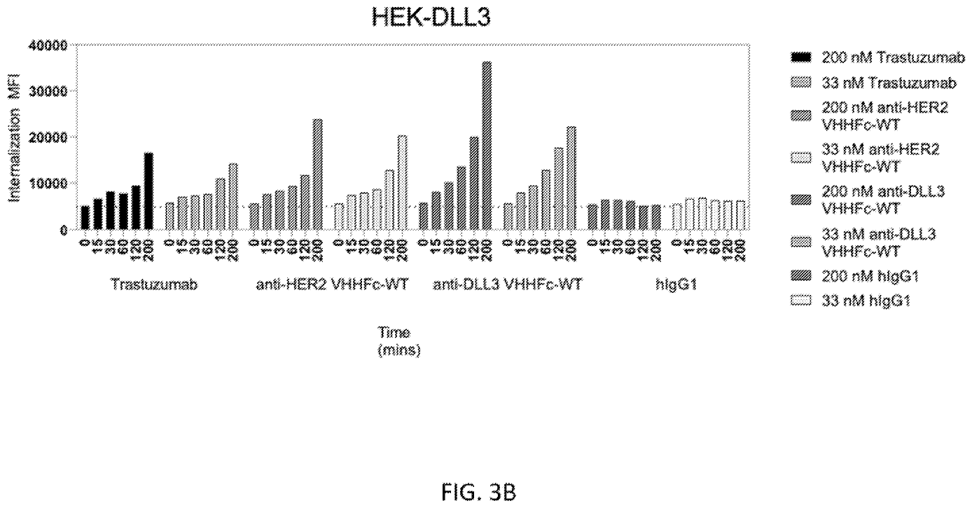

VHH-Fcs were tested for internalization by target-expressing cells using a secondary antibody conjugated to a pH sensitive dye. Goat anti-hu IgG-Fc secondary antibody was amine-conjugated to a pH sensitive pHAb dye (Promega Cat #G9845) according to the manufacturer's instructions. The pHAb dye has low or no fluorescence at pH>7 but fluoresces in acidic environment upon antibody internalization. Target-positive cells and target-negative cells were plated at 1.0×106/mL in a 96-well V bottom plate. VHH-Fcs and hIgG1 isotype control were diluted in media to 75 nM. Cells were spun to remove supernatant, resuspended with the prepared primary antibodies and incubated on ice for 1 hour. Excess primary antibody was washed off from cells and then incubated with pHAb labelled secondary antibody on ice for 30 minutes. Excess secondary was then washed off and cells were resuspended in media. One set of samples was placed in an incubator at 37° C. to allow internalization, and another set was left on ice (0° C.) as a binding only control. Cells were sampled at different time points ranging from 0 to 24 hours. Cells were stained with DAPI and read by flow cytometry on 572/28 channel with iQue screener platform. The VHH-Fcs show higher fluorescence than the negative controls (isotype, buffer) on target-positive cells. FIGS. 3A and 3B show that H101 and were D102 internalized by SHP-77 and HEK-DLL3 cells.

### Example 4. Antibody Thermal Stability Determination

Denaturing temperatures (Tm) of VHH-Fcs were determined from differential scanning fluorimetry (DSF) using Protein Thermo Shift Dye Kit™ (ThermoFisher, Cat #: 4461146). Briefly, A total of 1 g of antibody was used in each reaction. Melting curves of the antibodies were generated using an Applied Biosystems QuantStudio 7 Flex Real-Time PCR System with the recommended settings stated in the kit manual. The Tm's of the antibodies in Table 1 were then determined by using the ThermoFisher Protein Thermal Shift software (v.1.3). Tm1 of the VHH-Fcs was determined by DSF. Both H101 and D102 showed good thermostability of 67.5±0.1 Celsius. Additional, VHH-Fcs comprising mutations in the Fc region to decrease effector function and/or FcRn binding were tested for thermostability and resulted in slightly lower thermostability (1 to 2 degrees Celsius), but were still within acceptable ranges.

### Example 5. Receptor Density Determination

In order to test efficacy of the immunoconjugate binding with respect to target density receptor density was measured on target positive cell lines. Target density was measured using the ABC (Antibody Binding Capacity) assay. Cancer cells expressing the target of interest, as well as a negative control cell line, were harvested with cell dissociation buffer, seeded at about 5×104 cells per well into 96-well V bottom plate (Sarstedt 82.1583.001). Cells were tested for receptor expression using QuantiBRITE PE beads (BD Cat #340495) and a PE-conjugated anti-hu IgG (Biolegend clone HP6017) following the manufacturers' instructions. In brief, VHH-Fc and isotype control antibodies were prepared at suitable saturating concentrations based on previous experiments. Antibody sample dilutions were incubated with the panel of cell lines on ice for 1 hour. Cells were washed twice with 1% FBS in 1×PBS (FACS buffer), centrifuged at 400 G for 4 min. Cells were then incubated with 4 μg/mL mouse PE-conjugated anti-hu and DAPI (1:1000) for 30 minutes on ice. Cells were washed twice with FACS buffer, centrifuged at 400 G for 4 minutes and resuspended in FACS buffer. Fluorescence intensity on the PE channel was measured on the iQue Screener platform, and data were processed with ForeCyt software. The amount of PE signal generated from the different primary antibody was then fit to a standard curve based off of known PE molecules/Quantibrite bead samples to determine the number of antibody-binding sites per cell. Relative antibody binding sites correlate to the number of antigens or receptors on cell surface. Table 2 shows receptor density numbers for anti-DLL3 and anti-HER2 VHH-Fcs binding to a panel of cancer cell lines and were similar ranges to those reported in literature.

TABLE 2
Estimated number of epitopes/cell for each binder and cell line

| | | | HEK- | | | HEK293- | HCT- |
|---|---|---|---|---|---|---|---|
| | | SHP-77 | DLL3 | BT474 | H82 | 6E | 116 |
| Anti-DLL3 | Rova | 969 | 1679 | — | 936 | — | — |
| | D102 | 807 | 1734 | — | 794 | — | — |
| Anti-HER2 | Tmab | 625 | 1575 | 356690 | — | 1969 | 2790 |
| | H101 | 572 | 1490 | 401604 | — | 1935 | 2604 |

### Example 6. Affinity of Antibodies to Target Protein

Antibody affinity was assessed using Octet Red96e (ForteBio). The association rate constant (ka), dissociation rate constant (kd) and affinity constant (KD) were measured by biolayer interferometry with anti-hIgG Fc (AHC) capture biosensors (Fortebio cat #18-5063). Each cycle was performed with orbital shake speed of 1,000 rpm. Antigen was titrated 1:2 from a suitable starting concentration in kinetics buffer (Fortebio, Cat #18-1105). A set of AHC biosensors was dipped in kinetics buffer for baseline step of 60s. Anti-Target VHH-Fc (5 μg/mL, in kinetics buffer) was loaded onto the biosensors for 240 s followed by a second baseline step of 30 s. The IgG captured sensors were dipped into buffer for single reference subtraction to compensate natural dissociation of capture IgG. Each biosensor was then dipped into corresponding concentration of target protein (human, murine or cynomolgus monomeric protein) for 600 s, followed by 1800 s of dissociation time in kinetics buffer, or conditions as optimized. A new set of AHC biosensors was used for every VHH-Fc. The data was analysed by global fit 1:1 model for the association and dissociation step, (Octet software version v11.0). Table 3 shows binding affinity data.

TABLE 3
Affinity of H101 and D102 to target proteins

| VHH-Fc | Analyte | KD (nM) |
|---|---|---|
| D102 | Human DLL3-Flag | 0.472 |
| D102 | Mouse DLL3-His | 8.75 |
| H101 | Human HER2-His | 3.79 |

### Example 7. FcRn and Fc Effector Mutation Affinity Determination

FcRn affinity of VHH-Fc can generally be used to predict the half-life of antibody serum clearance. (See, e.g., Datta-Mannan A et al. “FcRn affinity-pharmacokinetic relationship of five human IgG4 antibodies engineered for improved in vitro FcRn binding properties in cynomolgus monkeys.” Drug Metab Dispos. 2012 August; 40(8):1545-55). Briefly, 10 nM of biotinylated hFcRn (Sino Biological, Cat #: CT071-H27H-B) was captured with the SA biosensor using Octet RED96e (Fortebio). The hFcRN coated biosensor was dipped into the sample solutions in sodium phosphate buffer (100 mM Na2HPO4, 150 mM NaCl w/0.05% Tween-20, pH 6.0) with serial concentrations of tested antibodies and the association measured. The dissociation was measured by dipping the biosensors into sodium phosphate buffer without antibody. The KD values were determined using Octet Data Analysis HT 11.0 software. 2:1 (Heterogeneous Ligand) binding model was used in analysis. Table 4 shows FCRN affinity for wildtype VHH-Fcs, and the impact of specific mutations in the Fc on affinity for the mutants. Changes in FcRn affinity were consistent across targets. Constructs with Fc Effector mutation only have no impact on FcRn affinity. Addition of Fc Effector mutations to FcRn mutation constructs does not affect FcRn affinity. Table 4a shows affinities of VHH-Fcs and Fc variants to FcRn.

TABLE 4a
Affinity of FcRn VHH-Fcs and Fc variants to FcRn

| VHH.Fc | FcRn Mutant | KD (nM) |
|---|---|---|
| H101 | wt | 3.7 |
| D102 | wt | 3.8 |
| H105 | I253A | Weak |
| H106 | S254A | 13 |
| H107 | H310A | No binding |
| H108 | H435Q | Weak |
| H109 | Y463A | 13 |
| H110 | H310A/H435Q | No binding |
| D111 | I253A | Weak |
| D112 | S254A | 19 |
| D113 | H310A | No binding |
| D114 | H435Q | Weak |
| D115 | Y463A | 20 |
| D116 | H310A/H435Q | No binding |
| H133 | wt | 2.1 |
| D134 | wt | 1.9 |
| H135 | H310A | No binding |
| D136 | H310A | No binding |
| H137 | H435Q | Weak |
| D138 | H435Q | Weak |

VHH-Fcs were also tested for affinity to FcTRs by biolayer interferometry using the Octet Red96e platform. Each cycle is performed with orbital shake speed of 1,000 rpm. Streptavidin (SA) biosensors (Sartorius 18-5019) were rehydrated for 10 mins using kinetics buffer (PBS+0.1% BSA+0.02% Tween-20). Biotinylated-FcTRs (Acro Biosystems) were then loaded for 40-100 s onto SA biosensors at concentrations ranging between 1-5 μg/mL diluted in PBS. VHH-Fcs were serially diluted 1:2 in sample buffer (PBS+0.02% Tween-20) with starting concentrations ranging between 5000 nM to 37.5 nM. Loaded biosensors were then associated with VHH-Fcs for 60-120 s. VHH-Fc dissociation was measured for 30-900 s in sample buffer. Bound VHH-Fcs were then removed using 3 cycles of 5 s regeneration buffer (150 mM NaCl, 300 mM Sodium Citrate) and 5 s sample buffer. The data was analyzed either using a globally-fitted 1:1 Langmuir binding model (FcγRI) or steady state analysis (Octet software version HT v11.1).

Analysis shows reduction in binding (represented by a higher KD) to FcγRs for constructs with those mutations incorporated as shown in Table 4b.

TABLE 4b
Affinity of FcRn VHH-Fcs and Fc variants to Fc receptors

| | | | FcγRIIa | FcγRIIa | | FcγRIIIa | FcγRIIIa |
|---|---|---|---|---|---|---|---|
| | Fc | FcγRI | (H167) | (R167) | FcγRIIb/c | (F176) | (V176) |
| | mutation | nM KD | nM KD | nM KD | nM KD | nM KD | nM KD |
| Trastuzumab | wt | 0.92 | 270 | 520 | 3700 | 630 | 110 |
| H101 | wt | 1.01 | 340 | 160 | 450 | 1600 | 480 |
| H133 | AEASS | — | — | 2300 | weak | — | — |
| H135 | AEASS + | — | — | 1200 | weak | — | — |
| | H310A | | | | | | |
| H137 | AEASS + | — | — | 1200 | weak | — | — |
| | H435Q | | | | | | |
| D102 | wt | 1.27 | 390 | 530 | 430 | 1200 | 730 |
| D134 | AEASS | — | weak | 460 | 1100 | — | — |
| D136 | AEASS + | — | weak | 570 | 2200 | — | — |
| | H310A | | | | | | |
| D138 | AEASS + | | weak | 520 | 770 | — | — |
| | H435Q | | | | | | |

(—) indicates no binding detected

### Example 8. Self-Association Studies Using AC-SINS

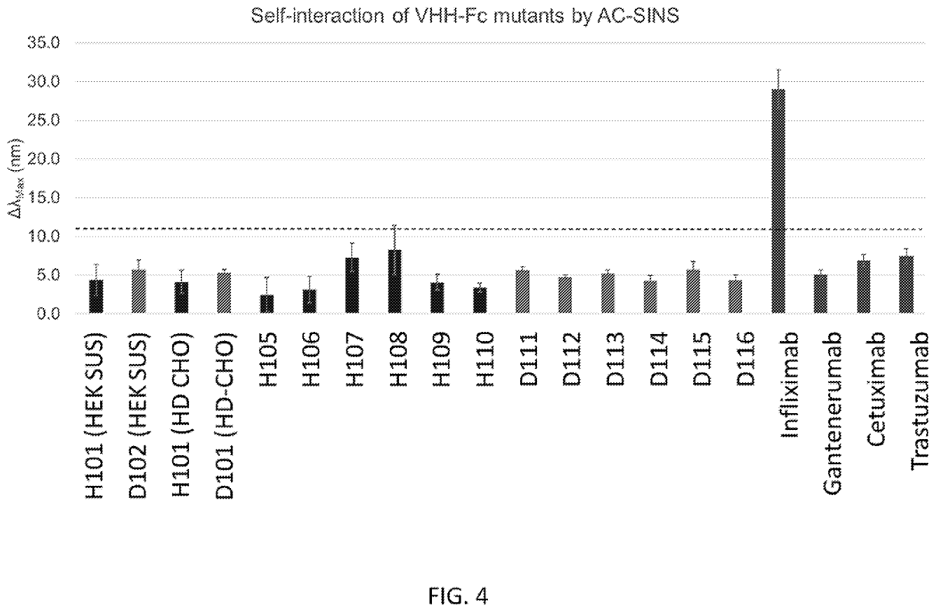

Propensities of self-association of VHH-Fcs was determined from affinity-capture self-interaction nanoparticle spectroscopy (AC-SINS) using gold nanoparticles (Au-NP) (Ted Pella, Cat #: 15705). (PMID: 24492294, 30395473) Briefly, goat IgG and goat anti-human Fc IgG (1:4 mole ratio) were used to coat the Au-NP. Conjugated Au-NP was mixed with 5 μg of each VHH-Fc, in quadruplicates, in a 96-well plate. The wavelength scan was measured with Synergy Neo2 plate reader. The difference of maximum absorbance (Δλmax) was calculated by subtracting λmax of each reaction with that of PBS buffer. The data was analyzed with Linest function in Excel using second-order polynomial fitting. Control antibodies with known high ACSINS score (above the literature established cut-off of 11 for IgGs) were included in the assay. FIG. 4 shows ACSINS scores for test articles and controls.

### Example 9. Polyreactivity Studies

Polyreactivity of VHH-Fcs against negatively charged biomolecules was determined by ELISA (As in Avery et al., “Establishing in vitro in vivo correlations to screen monoclonal antibodies for physicochemical properties related to favorable human pharmacokinetics.” MAbs. 2018 February/March; 10(2):244-255). Briefly, ELISA plate was coated with 5 μg/mL of human insulin (SigmaAlrich, Cat #: 19278) and 10 μg/mL of double stranded DNA (SigmaAlrich, Cat #: D1626-250MG) overnight. The plate was blocked with ELISA buffer (PBS, 1 mM EDTA, 0.05% Tween-20, pH 7.4). 10 μg/mL of test VHH-Fcs was loaded onto the plates in quadruplicates and incubated for 2 hours. Goat anti-human Fc(0.01 ug/ml) conjugated with HRP was then added and the plate incubated for 1 hour. The signal was developed with TMB and A450 absorbance was measured with Synergy Neo2 plate reader. The signal was normalized with the signal of non-coated well for each antibody tested. Table 5 shows the polyreactivity score, in comparison to control antibodies.

TABLE 5
Polyreactivity Assay Scores

| VHH.Fc | Insulin | dsDNA |
|---|---|---|
| H101 | 1.176 | 1.406 |
| D102 | 2.311 | 2.248 |
| H105 | 1.207 | |
| H106 | 1.321 | 1.446 |
| H107 | 1.306 | 1.678 |
| H108 | 1.420 | 1.663 |
| H109 | 1.244 | 1.579 |
| H110 | 1.181 | 1.317 |
| D111 | 2.202 | |
| D112 | 3.461 | 2.970 |
| D113 | 2.829 | 2.594 |
| D114 | 3.161 | 3.015 |
| D115 | 2.503 | 2.252 |
| D116 | 2.446 | 2.302 |
| Gantenerumab | >10 | >10 |

### Example 10. Fc Variants Effectively Reduce VHH-Fc Half-Life

In certain instances, reducing the drug half-life of alpha emitters is important for safety and to avoid unwanted toxicity associated with treatment. However, antibodies generally have a half-life upwards of 14 days or greater. Therefore, the half-life of the VHH-Fc variants was tested in order to observe and measure any reductions in half-life.

Twenty eight (28) 8 week old male B6.Cg-Fcgrttm1Dcr Tg(FCGRT)32Dcr/DcrJ (Tg32 hom, JAX stock #014565) mice were distributed into 7 groups with 4 mice per group as outlined in the table. Tg32 mice comprise a humanized FcRn and are generally viewed as a surrogate for human pharmacokinetics of antibodies when compared to non-human primates. (See, e.g., Avery L B et al. “Utility of a human FcRn transgenic mouse model in drug discovery for early assessment and prediction of human pharmacokinetics of monoclonal antibodies.” MAbs. 2016 August-September; 8(6):1064-78). On Day 0, body weights were measured and test articles were IV administered to all mice at 3 mg/kg and 5 ml/kg. 25 μL blood samples were collected from each mouse at time intervals. The blood samples were collected into 1 μL K3EDTA, processed to plasma, diluted 1/10 in 50% glycerol in PBS, transferred into specialized 96 well storage plates, and stored at −20° C. All plasma samples were assessed via a hIgG ELISA chosen for its high sensitivity for all seven test articles.

TABLE 6
Pharmacokinetic parameter summary for HER2 VHH-Fc

| | | | | | Volume of |
|---|---|---|---|---|---|
| | Terminal | Clear- | | | Distri- |
| | Half-Life | ance | Cmax | AUC | bution |
| | days | mL/days | μg/mL | μg-days/mL | mL |
| H105 | 1.12 | 152.1 | 63.9 | 841 | 137 |
| sem | 0.03 | 3.4 | 3.6 | 29 | 1 |
| H106 | 7.10 | 19.8 | 53.5 | 2193 | 177 |
| sem | 0.31 | 0.8 | 0.4 | 29 | 3 |
| H107 | 0.41 | 304.4 | 62.4 | 516 | 82 |
| sem | 0.01 | 15.0 | 4.3 | 22 | 3 |
| H108 | 1.57 | 117.5 | 46.6 | 903 | 174 |
| sem | 0.10 | 6.6 | 0.7 | 40 | 6 |
| H109 | 6.92 | 18.2 | 52.2 | 2519 | 152 |
| sem | 0.34 | 0.6 | 0.8 | 28 | 4 |
| H101 | 6.91 | 28.7 | 57.0 | 1946 | 218 |
| sem | 0.77 | 5.2 | 1.6 | 231 | 35 |
| trastuzumab | 14.54 | 5.9 | 59.0 | 4108 | 108 |
| sem | 1.12 | 0.5 | 2.3 | 109 | 2 |

As observed in Table 6, the introduction of mutations within the FcRn was generally able to reduce the half-life of the anti-HER2 VHH-Fc. Interestingly, contrary to published results in the field, not all Fe variants when included in the immunoconjugates tested showed a reduction in half-life consistent with previously published results found in the literature. (See, e.g., Burvenich I J et al., “Cross-species analysis of Fc engineered anti-Lewis-Y human IgG1 variants in human neonatal receptor transgenic mice reveal importance of S254 and Y436 in binding human neonatal Fc receptor.” MAbs. 2016 May-June; 8(4):775-86).

TABLE 7
Pharmacokinetic summary for DLL3 VHH-Fc

| | Terminal Half-Life | Clearance |
|---|---|---|
| | days | mL/days |
| D111 | 10.2 | 10.8 |
| SEM | 4.4 | 10.5 |
| D112 | 14.2 | 7.8 |
| SEM | 3.2 | 1.0 |
| D113 | 1.1 | 254.8 |
| SEM | 0.2 | 27.0 |
| D114 | 2.5 | 46.9 |
| SEM | 0.1 | 3.6 |
| D115 | 11.0 | 6.9 |
| SEM | 13.2 | 1.1 |
| D102 | 13.3 | 10.3 |
| SEM | 3.3 | 3.8 |
| trastuzumab | 18.4 | 3.7 |
| SEM | 5.9 | 0.9 |

As observed in Table 7, the introduction of mutations within the FcRn was generally able to reduce the half-life of the anti-DLL3 VHH-Fc. Similarly to HER2 binding immunoconjugates and contrary to published results, not all Fc variants showed a reduction in half-life consistent with previously published results found in the literature.

### Example 11. VHH-Fc Intact Mass Analysis

Conjugates were deglycosylated prior to analysis with in-house Endo-S enzyme (final concentration of 10 μg/mL) at 37° C. for 1 hour.

For analysis of the intact mass, 8 μL samples were injected on a Waters Acquity UPLC-Q-TOF with a UPLC BEH200 SEC 1.7 μM 4.6×150 mm column. These samples were eluted with a mobile phase of water/ACN (70/30, v/v) with 0.1% TFA and 0.1% FA (formic acid) for 11 min with a flow rate of 0.25 mL/min.

### Example 12. Sourcing Bifunctional Chelators

Several chelators are known to practitioners of the art which are pre-functionalized for antibody conjugation. p-SCN-Bn-DOTA (1) is available from Macrocyclics (Plano, TX). Other linker variations of DOTA can be produced from the advanced intermediate DOTAGA-tetra(t-Bu ester) (2) (Macrocyclics, Plano, TX) following the general procedure below.

Other reagents used in these procedures are available from Millipore Sigma, CombiBlocks, Chem-Impex, and Broadpharm. All solvents were obtained from VWR and used as is with no anhydrous handling conditions unless indicated. Mass spectra were taken with an Agilent HPLC-MS or Waters HPCS-MS with C18 reverse phase column and an acetonitrile/water (+0.1% formic acid) gradient. Flash chromatography was performed using a Biotage IsoleraOne instrument with an appropriately sized normal phase silica gel cartridge with fraction collection at 254 nm. Final compounds were purified by an Agilent prep-scale HPLC using an acetonitrile/water (+0.1% TFA) gradient. NMR spectra were taken with a Bruker 400 MHz NMR instrument and processed with MestReNova v.14. Detailed NMR Data was compiled with the multiplet analysis function used in manual mode.

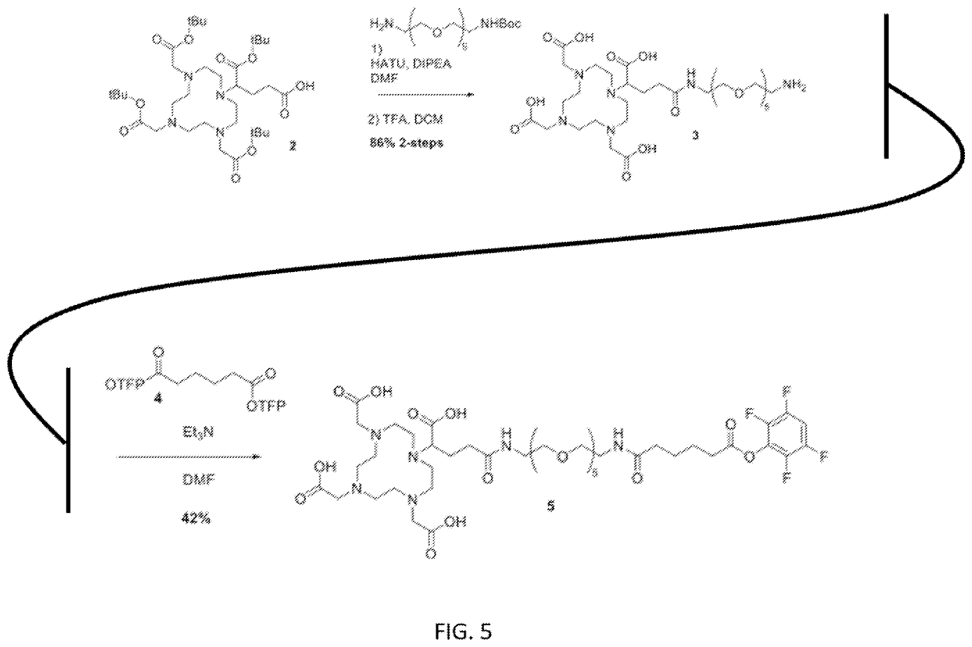

FIG. 5 shows PEG5-DOTA synthesis, including compounds numbered (2)-(5), as described below. Compound 3 was prepared through a HATU coupling, followed by TFA deprotection. Available without chromatographic purification.

Synthesis of Compound (3) 4-({2-[2-(2-aminoethoxy)ethoxy]ethyl}carbamoyl)-2-[4,7,10-tris(carboxymethyl)-1,4,7,10-tetraazacyclododecan-1-yl]butanoic acid; tetrakis (trifluoroacetic acid): Compound 2 (100 mg, 0.143 mmol) was taken up in DMF (2 mL), HATU (65.1 mg, 0.171 mmol) was added, then DIPEA (0.099 mL, 73.8 mg, 0.57 mmol) was added. After 3 min, a solution of Boc-NH-PEG5-amine (65.1 mg, 0.17 mmol), was added to the reaction. After stirring for 10 min, HPLC showed the reaction to be complete. After 1 h, the reaction was quenched with about 5 mL NaHCO₃(sat), then 5 mL of water was added and the mixture was extracted 4×30 mL Et2O. The combined organics were washed with saturated brine, dried over sodium sulfate, filtered, and concentrated in vacuo to yield the crude protected intermediate in good purity. m/z found=1063.6 (M+H).

The above intermediate was directly taken up in DCM (5 mL) and TFA (5 mL) was added. The reaction was stirred for 24 h until HPLC indicated complete removal of Boc and tBu esters. The reaction solution was concentrated in vacuo and co-evaporated 2× with 25 mL DCM. The residue was precipitated from DCM with Et2O, then the remaining solid was triturated extensively with sonication (15-30 min) to yield the title compound (128 mg, 86% two-steps) as an off-white powder in good purity. 1H NMR (400 MHz, Deuterium Oxide) δ 4.15-3.68 (m, 7H), 3.62 (d, J=4.7 Hz, 2H), 3.59-3.49 (m, 20H), 3.47 (t, J=5.5 Hz, 2H), 3.35-2.78 (m, 16H), 2.52-2.37 (m, 2H), 1.97-1.79 (m, 2H). m/z found=739.5 (M+H).

Synthesis of Compound (4) Bis(2,3,5,6-tetrafluorophenyl) hexanedioate: Adipic Acid (1.00 g, 6.84 mmol) and EDC (3.28 g, 17.1 mmol) were taken up in 20 mL DCM and cooled to OC in an ice bath, then a solution of 2,3,5,6-tetrafluorophenol in 20 mL DCM was added. Conversion to product was observed by TLC (Rf=0.5; 75% DCM/Hexanes). The reaction mixture was concentrated in vacuo and purified by flash chromatography (0-100% DCM/Hexanes) to yield the title compound (2.48 g, 82%) as a crystalline white powder. 1H NMR (400 MHz, Chloroform-d) δ 7.03 (tt, J=9.9, 7.0 Hz, 2H), 3.00-2.63 (m, 4H), 1.95 (t, J=3.3 Hz, 4H). This compound has poor signal by LCMS.

Compound (5) -{[2-(2-{2-[6-oxo-6-(2,3,5,6 tetrafluorophenoxy) hexanamido]ethoxy}ethoxy)ethyl]carbamoyl}-2-[4,7,10-tris(carboxymethyl) 1, 4, 7, 10 tetraazacyclododecan-1-yl]butanoic acid: To a solution of compound 3 (22.1 mg, 0.017 mmol) in DMF (1.5 mL) was added bis(2,3,5,6-tetrafluorophenyl) hexanedioate (4) (45.2 mg, 0.102 mmol) and triethylamine (0.0086 mL, 6.2 mg, 0.061 mmol). Full conversion to product was confirmed by HPLC. After stirring for 2 h, the reaction was diluted with DMSO (1.5 mL) and purified by direct injection onto prep-HPLC (Agilent, Hanover, CT) with a gradient of 15-50% MeCN/water+0.1% TFA to yield the title compound (10.6 mg, 50%) as a white powder (2×TFA salt). 1H NMR (400 MHz, Deuterium Oxide) δ 7.20 (tt, J=10.4, 7.2 Hz, 1H), 3.97-3.65 (m, 5H), 3.58-3.51 (m, 20H), 3.49 (q, J=5.1 Hz, 2H), 3.43-3.32 (m, 6H), 3.26 (t, J=5.3 Hz, 2H), 3.20-2.82 (m, 12H), 2.69 (t, J=6.8 Hz, 2H), 2.52-2.34 (m, 2H), 2.19 (t, J=6.8 Hz, 2H), 1.99-1.82 (m, 2H), 1.75-1.46 (m, 4H). m/z found=1015.3 (M+H).

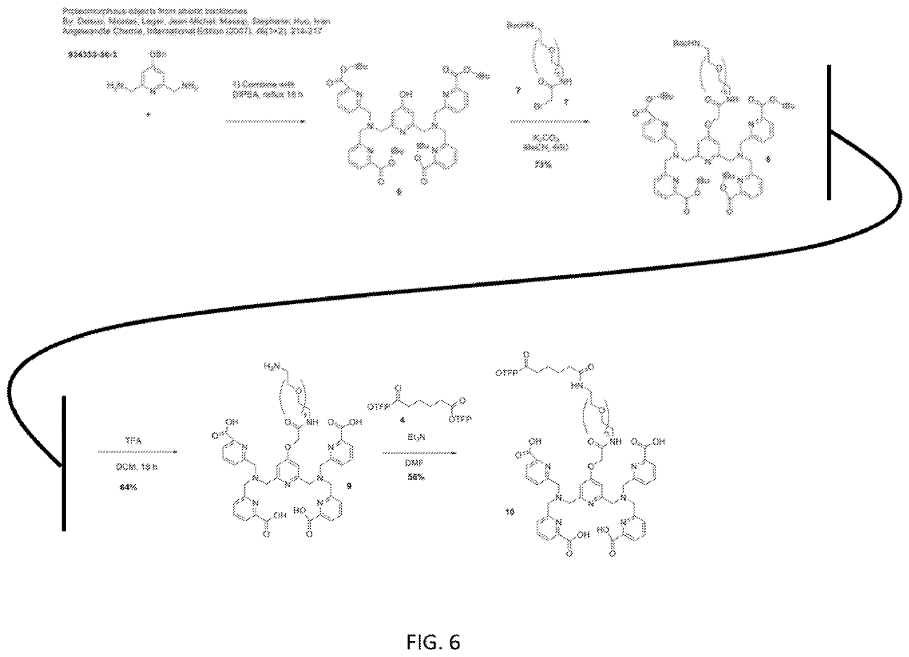

FIG. 6 shows PEG5-Py4Pa synthesis, including compounds numbered (6)-(10) as described below.

Synthesis of Compound (6) tert-butyl 6-[({[4-(benzyloxy)-6-{[bis({6-[(tert-butoxy)carbonyl]pyridin-2-yl}methyl)amino]methyl}pyridin-2-yl]methyl}({6-[(tert-butoxy)carbonyl]pyridin-2-yl}methyl)amino)methyl]pyridine-2-carboxylate. To a stirred solution of 1-[6-(aminomethyl)-4-(benzyloxy)pyridin-2-yl]methanamine (0.65 g, 2.67 mmol) (available from N. Delsuc, et al. Angew Chem. Int. Ed. 2007, 46, 214-217) in acetonitrile (50 mL) was added DIPEA (1.40 mL, 1.04 mg, 8.01 mmol) and tert-butyl 6-(bromomethyl)pyridine-2-carboxylate (4.36 g, 16.0 mmol) (available from P. Coomba, et al. Inorg. Chem. 2016, 55, 12531-12543) and the solution was heated to reflux. After 16 h, the reaction was allowed to cool and the solvent removed in vacuo. The crude was taken up in 200 mL DCM and washed 2×75 mL NaHCO₃(sat) and 2×75 mL saturated brine. The DCM layer was then dried over sodium sulfate, filtered, and concentrated in vacuo to yield a brown crude oil (950 mg) that could be used in the following step without further purification. The intermediate from above was dissolved in EtOH, ammonium formate (297 mg, 4.71 mmol) was added, and the flask was purged with N2. 10% Pd/C (250 mg, 0.23 mmol) was added followed by another purge with N2, then 30% Pd/C (50 mg, 0.14 mmol) was added. Following another purge with N2, the reaction was heated to 50C and stirred for 6 h where the reaction was complete by LCMS. The reaction mixture was filtered through celite, washed 3×50 mL MeOH, then concentrated in vacuo to a pale-yellow oil. The crude was purified by flash chromatography using a Biotage Sfar amino D cartridge and a gradient of 40-100% EtOAc/Hexanes followed by 0-20% MeOH/DCM to yield the title compound as a yellow solid (278 mg, 11%). 1H NMR (400 MHz, Methanol-d4) δ 7.88 (dd, J=7.7, 1.3 Hz, 4H), 7.82 (t, J=7.7 Hz, 4H), 7.73 (dd, J=7.7, 1.2 Hz, 4H), 6.41 (s, 2H), 4.00 (s, 8H), 3.94 (s, 4H), 1.61 (s, 36H). m/z found=918.4 (M+H).

Synthesis of Compound (7) tert-butyl N-[17-(2-bromoacetamido)-3,6,9,12,15-pentaoxaheptadecan-1-yl]carbamate: A solution of tert-butyl N-(17-amino-3,6,9,12,15-pentaoxaheptadecan-1-yl)carbamate (200 mg, 0.53 mmol) and DIPEA (0.146 mL, 109 mg, 0.84 mmol) in 5 mL DCM was cooled to 0° C. A solution of 2-bromoacetyl bromide (0.069 mL, 159 mg, 0.79 mmol) in 5 mL DCM cooled to 0° C. was added dropwise over 2 min. The reaction was allowed to warm to rt, after 90 min HPLC showed full conversion to product. The reaction was concentrated, partitioned between Et20 and water, NaHCO₃(sat) was added, then the mixture was extracted 3×25 mL with Et2O. The combined organics were washed with brine, dried over sodium sulfate, filtered and concentrated in vacuo. The crude residue was co-evaporated once with acetonitrile to remove water. The title compound was recovered as a brownish oil (261 mg, 99%). 1H NMR (400 MHz, Chloroform-d) δ 3.90 (s, 2H), 3.75-3.64 (m, 18H), 3.61 (d, J=4.5 Hz, 2H), 3.56 (t, J=5.1 Hz, 2H), 3.52 (t, J=5.2 Hz, 2H), 3.37-3.30 (m, 2H), 1.46 (s, 9H). m/z found=523.2 (M+Na).

Synthesis of Compound (8) tert-butyl 6-({[(6-{[bis({6-[(tert-butoxy) carbonyl]pyridin-2-yl}methyl)amino]methyl}-4-{[(17-{[(tert-butoxy)carbonyl]amino}-3,6,9,12,15-pentaoxaheptadecan-1-yl)carbamoyl]methoxy}pyridin-2-yl)methyl]({6-[(tert-butoxy)carbonyl]pyridin-2-yl}methyl)amino}methyl)pyridine-2-carboxylate. Compound 6 (100 mg, 0.11 mmol) and compound 7 (81.9 mg, 0.163 mmol) were taken up in acetonitirile (5 mL), then potassium carbonate (30.1 mg, 0.218 mmol) was added and the reaction was stirred at 60 C. After 24 h, no starting material remained by HPLC. The reaction was concentrated and purified by flash chromatography (Biotage amino D cartridge, gradient 0.2-15% MeOH/DCM) to yield the title compound as a yellow film (106 mg, 73%). 1H NMR (400 MHz, Methanol-d4) δ 7.89 (d, J=7.8 Hz, 4H), 7.83 (t, J=7.7 Hz, 4H), 7.66 (d, J=7.6 Hz, 4H), 6.95 (s, 2H), 4.66 (s, 2H), 4.04 (s, 8H), 3.92 (s, 4H), 3.75-3.55 (m, 20H), 3.53-3.43 (m, 2H), 3.30-3.13 (m, 2H), 1.52 (s, 36H), 1.43 (s, 9H). m/z found=670.0 (M+2H/2).

Synthesis of Compound (9) 6-({[(4-{[(17-amino-3,6,9,12,15-pentaoxaheptadecan-1-yl)carbamoyl]methoxy}-6-({bis[(6-carboxypyridin-2-yl) methyl]amino}methyl)pyridin-2-yl)methyl][(6-carboxypyridin-2-yl)methyl]amino}methyl) yridine-2-carboxylic acid: Compound 8 (125 mg, 0.093 mmol) was taken up in DCM (5 mL) and TFA (5 mL) was added. After 18 h, HPLC showed no starting material or t-butyl intermediates remaining. The reaction was concentrated in vacuo and co-evaporated once with DCM. The crude oil was triturated 2× with Et2O with sonication and collected by filtration to yield 100 mg (64%, as a 5×TFA salt) of the title compound as a brownish solid. 1H NMR (400 MHz, Methanol-d4) δ 8.04 (d, J=7.7 Hz, 4H), 7.96 (t, J=7.8 Hz, 4H), 7.66 (t, J=8.4 Hz, 4H), 7.45 (s, 2H), 4.84 (s, 2H), 4.74-4.49 (m, 12H), 3.74 (t, J=5.0 Hz, 2H), 3.71-3.63 (m, 14H), 3.60 (t, J=5.3 Hz, 2H), 3.48 (t, J=5.6 Hz, 2H), 3.20-3.12 (m, 2H). m/z found=1014.3 (M+H).

Synthesis of Compound (10) 6-[({[6-({bis[(6-carboxypyridin-2-yl)methyl]amino}methyl)-4-[({17-[6-oxo-6-(2,3,5,6-tetrafluoro-phenoxy) hexanamido]-3,6,9,12,15-pentaoxaheptadecan-1-yl}carbamoyl)methoxy]pyridin-2-yl]methyl}[(6-carboxypyridin-2-yl)methyl]amino)methyl]pyridine-2-carboxylic acid. To a solution of compound 9 (80 mg, 0.079 mmol) in DMF (2.5 mL) was added bis(2,3,5,6-tetrafluorophenyl) hexanedioate (4) (140 mg, 0.32 mmol) and triethylamine (0.027 mL, 20 mg, 0.197 mmol). Full conversion to product was confirmed by HPLC. After stirring for 4 h, the reaction was diluted with DMSO (1.5 mL) and purified by direct injection onto prep-HPLC (Agilent, Hanover, CT) with a gradient of 25-60% MeCN/water+0.1% TFA to yield the title compound (57.5 mg, 56%) as a white powder (3×TFA salt). 1H NMR (400 MHz, Deuterium Oxide) δ 7.85 (t, J=7.8 Hz, 4H), 7.78 (dd, J=7.8, 1.2 Hz, 4H), 7.50 (dd, J=7.8, 1.2 Hz, 4H), 7.11 (tt, J=10.4, 7.2 Hz, 1H), 6.99 (s, 2H), 4.59 (s, 2H), 4.49 (s, 8H), 4.45 (s, 4H), 3.60-3.45 (m, 18H), 3.46 (t, J=5.3 Hz, 2H), 3.36 (t, J=5.3 Hz, 2H), 3.22 (t, J=5.3 Hz, 2H), 2.59 (t, J=6.7 Hz, 2H), 2.14 (t, J=6.7 Hz, 2H), 1.61-1.46 (m, 4H). m/z found=1290.3 (M+H).

Synthesis of Compound (11) 6-[({[6-({bis[(6-carboxypyridin-2-yl)methyl]amino}methyl)-4-{2-[4-(cyanosulfanyl)phenyl]ethoxy}pyridin-2-yl]methyl}[(6-carboxypyridin-2-yl)methyl]amino)methyl]pyridine-2-carboxylic acid; bis(tri-fluoroacetic acid): The title compound was prepared by following the conditions in L Li et al. Bioconjugate Chem. 2021, 32, 1348-1363. Spectral and LCMS data matched reported values.

### Example 13. Conjugation of VHH-Fc Proteins with Chelator-Linkers

Conjugations can be carried out using many of the methods available for preparation of IgG radioconjugates and IgG antibody-drug conjugates. For information on the range of applicable methodologies, see PW Howard Antibody-Drug Conjugates (ADCs), Protein Therapeutics, First Edition, chapter 9, pp. 278-279 (2017).

For a typical lysine-based conjugation, a VHH-Fc was buffer-exchanged into 0.1 M NaHCO-3, pH 8.5-9.5 by either Microsep Advance Centrifugal Device (Pall 10K MWCO, Cat #: MCP010C41) or by Zeba column (ThermoFisher, Cat #: 87768), followed by sterilization with a Costar Spin-X Centrifuge Tube, 0.22 m (Corning, Cat #: 8160). The buffer-exchanged antibody was quantified by BCA assay. An appropriate molar excess (5-20 eq) of chelator-linker (50 mM in DMSO) was added to the VHH-Fc (2 mg/mL final concentration) and the reaction was incubated at 25° C. either for 2 h or overnight in the Thermomixer. After the reaction was complete, the sample was passed through a Zeba column (ThermoFisher, Cat #: 87770) according to the manufacturer's protocol to remove unused chelator-linker and buffer-exchange into PBS (pH 7.4) (LifeTechnologies, Cat #: 10010-023). This VHH-Fc-chelator conjugate (VFCC) was stored at 4° C. until analysis and purification.

### Example 14. VHH-Fc-Chelator Conjugate (VFCC) Purification with SEC

To remove high molecular weight species (HMWS) and low molecular weight species (LMWS), VHH-Fcs were purified by SEC using an AKTA Pure FPLC system with a Cytiva HiLoad 16/600 Superdex 200 μg column. TBS buffer (50 mM Tris, 150 mM NaCl, OmniTrace Ultra water [VWR, Cat #: CAWX0003-2]), pH 7.6 was used for the SEC buffer. The fractions containing intact VHH-Fcs were pooled together and concentrated using Microsep Advance Centrifugal Device (Pall 10k MWCO, Cat #: MCP010C41). The concentrated sample was transferred to an Ultrafree-MC GV Centrifugal Filter, 0.22 μm 0.5 mL (Millipore, Cat #: UFC30GV0S) and spun at 3,000×g for 3 minutes.

### Example 15. Protein Quantification

VHH-Fc protein content was quantified with a Pierce BCA Protein Assay Kit (Thermo, Cat #: 23225) standardized by Cetuximab (LIST/E: 094822, DIN 02271249, 2 mg/mL).

### Example 16. Chelator to VHH-Fc Ratio (CAR) Analysis

The chelator loading ratio, herein described as CAR, can be analyzed through methods applicable to practitioners of the art of antibody conjugates. For a review of these methods in the context of ADCs, see A Wakankar et al., mAbs 3:161 (2011). The CAR of each conjugate was analyzed by DG-SEC-MS.

Conjugates were analyzed through the deglycosylation and UPLC-Q-TOF procedure described in Example 11. In this case, a distribution of masses is obtained after spectrum deconvolution that allows calculation of the average CAR of the preparation.

Conjugates were analyzed through the deglycosylation and UPLC-Q-TOF procedure described in Example 11. In this case, a distribution of masses is obtained after spectrum deconvolution that allows calculation of the average CAR of the preparation.

### Example 17. Binding of VHH-Fc Conjugates to Cells Expressing Target Protein

In some instances, conjugation can negatively impact binding of the VHH-Fc to the target protein. Binding of VHH-Fc conjugates was therefore tested, similar to as described above. Table 8 shows cell binding data of VHH-Fc chelator conjugates.

TABLE 8
Cell binding data of VHH-Fc chelator conjugates
EC50 (nM)

| | Antibody | SHP-77 | HCT-116 | HEK-DLL3 | HEK-293 |
|---|---|---|---|---|---|
| Controls | Rovalpituzumab | 0.11 | — | 0.06 | — |
| | Trastuzumab | — | 1.16 | 1.06 | 0.69 |
| | hIgG1 | — | — | — | — |
| Short | H101 (CAR 0) | — | 2.21 | 1.62 | 1.14 |
| Linker DOTA | H101 (CAR 0.6) | — | 1.96 | 1.73 | 1.16 |
| p-SCN- | H101 (CAR 1.1) | — | 2.46 | 1.39 | 1.42 |
| Bn-DOTA | H101 (CAR 2.3) | — | 3.34 | 2.05 | 1.68 |
| | H101 (CAR 2.7) | — | 2.96 | 1.88 | 1.58 |
| | H101 (CAR 4.6) | — | 5.63 | 2.99 | 2.27 |
| | H101 (CAR 8.3) | — | 5.32 | 4.43 | 3.52 |
| | D102 (CAR 0) | 0.53 | >100 | 1.42 | >10 |
| | D102 (CAR 0.9) | 0.41 | — | 0.48 | — |
| | D102 (CAR 4.7) | 0.38 | — | 0.56 | — |
| Long | H101 (CAR 0) | — | 2.21 | 1.62 | 1.14 |
| Linker DOTA | H101 (CAR 2.0) | — | 4.01 | 3.99 | 3.11 |
| TFP-Ad- | H101 (CAR 8.9) | — | 40.11 | 28.37 | 28.89 |
| PEG5-DOTA | D102 (CAR 0) | 0.53 | >100 | 1.42 | >10 |
| | D102 (CAR 2.7) | 0.50 | — | 0.58 | — |
| | D102 (CAR 9.3) | 0.60 | — | 0.91 | — |

H101 = Her2 antigen binding;
D102 = DLL3 antigen binding;
CAR = Chelator to VHH ratio

As observed in Table 8, binding was observed for both long and short DOTA linkers. As also shown in Table 8, binding was also observed across increasing chelator VHH-Fc ratios (CAR).

### Example 18. Percent Intact Analysis

The percent intact immunoconjugate was established by HPLC-SEC. 12 μL of conjugate was added to a glass vial insert in a standard HPLC vial. 10 μL of sample was injected onto an Agilent HPLC-SEC with a Wyatt Technology WTC-050S5 SN:0429 BN WBD129 column column and eluted with 1×PBS (100%) for 40 min at a flow rate of 0.5 mL/min

### Example 19. Endotoxin Level Determination

Endotoxin test was performed using Wako's Limulus Amebocyte Lysate Pyrostar™ ES—F Single Test (Cat #: WPESK-0015) according to manufactural protocol. The QC cutoff was set based on the maximum injection dose projected for each animal in the study while following appropriate animal care and FDA guidelines.

### Example 20. Radiolabeling with In-111

40 μg of each of the 4 test articles was diluted to 100 μL with 0.1 M ammonium acetate buffer in a 500 μL lo-bind Eppendorf tube and 18-25 μL (20-22 MBq) of [111In]InCl3 was added and mixed with a pipette. The reaction mixtures were incubated at 37° C. in an incubator for 1 hour. The tubes were then transferred to a 4° C. fridge.

Incorporation of radionuclides was determined by spotting 0.5 μL of sample at the origin of a 1.5×10 cm iTLC strip. The strip was then placed in a 50 mL Falcon tube containing 2 mL of mobile phase (25 mM EDTA in pH 5 0.1 M sodium acetate buffer) until the solvent had reached the top of the strip. The strip was removed and exposed to a phosphor imaging plate which was then scanned in a Cyclone phosphor imager. Regions of interest were drawn over spots corresponding to the migration of protein-bound and un-bound In-111 and the proportion in each calculated.

Radioconjugates were also analyzed by SEC-HPLC: A volume corresponding to 0.1-0.2 MBq of the sample was pipetted into a 500 μL lo-bind Eppendorf tube and the radioactivity measured in an ionization chamber. The sample was drawn up into a syringe and injected onto the HPLC system. Samples were eluted with PBS. The eluate from the system was collected and the radioactivity measured in order to determine the recovery from the column (corrected for activity remaining in the sample tube and the injection syringe).

TABLE 9
Indium-111 Radiolabeling Efficiency

| | | Labelling efficiency post-synthesis | |
|---|---|---|---|
| Chelator-Linker | Antibody | Attempt 1 | Attempt 2 |
| P-SCN-Bn-DOTA | H101 | 95.9% | 96.5% |
| TFP-Ad- | | 97.5% | 97.7% |
| PEG5-DOTAGA | | | |
| P-SCN-Bn-DOTA | D102 | 97.5% | 97.0% |
| TFP-Ad- | | 98.1% | 97.2% |
| PEG5-DOTAGA | | | |

### Example 21. Radiolabeling with Ac-225

800 g of each of the 4 test articles was diluted to 200 μL with 0.2 M ammonium acetate buffer pH 6.5 in a 500 μL lo-bind Eppendorf tube and 2 μL (400 kBq) of 225-Actinium chloride was added and mixed with a pipette. The reaction mixtures were incubated at 37° C. in an incubator for 1 hour in the case of the Py4Pa conjugates and 2 hours for the DOTA conjugates. The tubes were then transferred to a 4° C. fridge.

Incorporation was measured by spotting 0.5 μL of sample at the origin of a 1.5×10 cm iTLC strip and allowing it to dry for a few minutes. The strip was then placed in a 50 mL Falcon tube containing 2 mL of mobile phase (25 mM EDTA in pH 5 0.1 M sodium acetate buffer) until the solvent had reached the top of the strip. The strip was removed and allowed to equilibrate for at least 2 hours, after which it was exposed to a phosphor imaging plate which was then scanned in a Cyclone phosphor imager. Regions of interest were drawn over spots corresponding to the migration of protein-bound and un-bound Ac-225 and the proportion in each calculated.

Alternately, samples could be assayed by HPLC-SEC: HPLC of DOTA conjugates used a BioSEP SEC 5 μm s3000 3007.88 mm column with 20% acetonitrile in PBS elution. HPLC of Py4Pa conjugates used a Wyatt 050S5 5 μm 500 A 7.8×300 mm column with 20% acetonitrile in PBS elution).

50 μL of each sample was drawn up into a Hamilton syringe and injected onto the HPLC system. From 10-30 minutes post injection, 30 second fractions of the eluate (0.25 mL) were collected by hand into counting tubes. The fractions were allowed to reach secular equilibrium for 24 hours and then measured in a gamma counter. A 5 μL sample of each preparation was also counted to enable the recovery from the HPLC system to be calculated. Radiochemical purity was determined by determining the area under the peak for 18.5-22.5 mins and 19.5-23.5 mins for DOTA and Py4Pa conjugates, respectively, as a percentage of total counts. As shown in Table 10 all chelator-linker combinations showed good labeling efficiency.

TABLE 10
Ac-225 Radiolabeling Efficiency

| | | iTLC Labelling efficiency |
|---|---|---|
| Chelator-Linker | Antibody | immediately after preparation |
| p-SCN-Bn-DOTA | H101 | 92.0% |
| TFP-Ad-PEG5-DOTAGA | | 96.3% |
| TFP-Ad-PEG5-Py4Pa | | 93.1% |
| p-SCN-Ph—Et—Py4Pa | | 96.0% |
| p-SCN-Bn-DOTA | D102 | 98.5% |
| TFP-Ad-PEG5-DOTAGA | | 99.5% |
| TFP-Ad-PEG5-Py4Pa | | 98.0% |
| p-SCN-Ph—Et—Py4Pa | | 100% |

### Example 22. Stability of VHH-Fc Radioconjugates

The stability of the radiolabeled immunoconjugates was tested, both for 225Ac and 111In. VHH-Fc chelator-conjugates were radiolabeled (either In-i 11 or Ac-225) as described above. For stability in PBS, 50 μL of each labelled test article was then added to either 200 μL of PBS (with In-ill) or 200 μL PBS/ascorbate (with Ac-225) and stored at 4° C. For stability in serum, 50 μL of each labelled test article was added to 200 μL of mouse serum and incubated at 37° C. Aliquots of were taken at different time points and analyzed for radiochemical purity using iTLC and/or HPLC-SEC as described above. The results of these stability experiments are shown in Table 11 and Table 12 below and indicated that the radio conjugates were stable in both PBS and serum.

TABLE 11
Stability of Her2 and DLL3 conjugates labeled with In-111

| | DLL3 (D102) | | HER2 (H101) | |
|---|---|---|---|---|
| Radiochemical | P-SCN- | TFP-Ad- | P-SCN- | TFP-Ad- |
| purity by | Bn- | PEG5- | Bn- | PEG5- |
| HPLC (iTLC) | DOTA | DOTAGA | DOTA | DOTAGA |
| PBS 1 h | 97.5% | 98.1% | 97.5% | 98.4% |
| PBS 24 h | 89.1% | 95.2% | 96.5% | 98.4% |
| Serum 24 h | 94% (94%) | 98% (94%) | 97% | 94% |
| Serum 72 h | 92% (92%) | 96% (94%) | 100% (87%) | 100% (84%) |
| Serum 168 h | 92% (94%) | 95% | 95% (91%) | 92% |

TLC radiochemical incorporation values presented in parentheses. iTLC incorporation >95% except where shown

TABLE 12
Stability of Her2 and DLL3 conjugates labeled with Ac-225

| | DLL3 (D102) | | | | HER2 (H101) | | | |
|---|---|---|---|---|---|---|---|---|
| | P- | TFP- | TFP- | P- | P- | TFP- | TFP- | P- |
| Radiochemical | SCN- | Ad- | Ad- | SCN- | SCN- | Ad- | Ad- | SCN- |
| purity by | Bn- | PEG5- | PEG5- | Ph-Et- | Bn- | PEG5- | PEG5- | Ph-Et- |
| HPLC (iTLC) | DOTA | DOTAGA | Py4Pa | Py4Pa | DOTA | DOTAGA | Py4Pa | Py4Pa |
| PBS 1 h | 91% | 92% | 83% | 82% | 93% | 93% | 84% | N/D |
| PBS 24 h | 92% | 92% | 83% | 83% | 93% | 91% | 82% | 82% |
| Serum 24 h | 88% | 91% | 78% | 69% | 91% | 90% | 75% | 68% |
| | (94%) | | | | | | | |
| Serum 72 h | 89% | 90% | 73% | 65% | 89% | 85% | 74% | 61% |
| | (90%) | (94%) | | | (87%) | (94%) | | |
| Serum 168 h | 81% | 86% | 71% | 59% | 85% | 80% | 70% | 56% |
| | (91%) | | | | (89%) | | | |

TLC radiochemical incorporation values presented in parentheses. iTLC incorporation >95% except where shown

### Example 23. Immunoreactivity of VHH-Fc Radioconjugates

The immunoreactive fraction (IRF) was determined though a method described by SK Sharma et al. in Nucl. Med. Biol. 2019, 71, 32-38. Samples were incubated overnight in PBS at 4° C. for analysis and before in vivo experiments, while some samples were incubated in serum at 37° C. for 3 and 7 days as an alternate measure of stability.

### Bead Coating

Dynabeads and antigen (0.15 nmol per 0.125 ug beads) were incubated in B/W buffer (25 μL/0.125 ug beads) at room temperature on a tube rotator for 30 minutes. The Eppendorfs were spun at 100×g for 15 seconds and placed on a magnetic rack for 3 minutes. The supernatant was removed and the beads washed with PBSF. 1 mg of beads was then resuspended in 200 μL of B/W buffer and 2 mg in 400 μL of B/W buffer. Control beads were prepared the same way, except with no antigen added to the tubes.

### Immunoreactive Fraction (IRF) Assay

The appropriate volume of beads (25 μL/0.125 mg beads) generated above was added to microcentrifuge tubes, prewashed with 1 mL PBSF. Radiolabeled VHH-Fc-conjugate (10 ng), block (10 or 50 ug unconjugated antibody; if required), and PBSF were added to each reaction to achieve a final volume of 350 μL. The samples were incubated at room temperature on a rotor for 30 minutes. After this the tubes were centrifuged at 100×g for 15 seconds and placed on a magnetic rack for 3 minutes. The supernatant was collected in a gamma counter tube. The beads were washed twice with 400 μL PBSF and collected in a separate gamma counter tube. The beads were finally resuspended in 500 μL PBSF and transferred to a gamma counter tube. The reaction tube was washed with 500 μL PBSF and this was added to the gamma counter tube containing the beads.

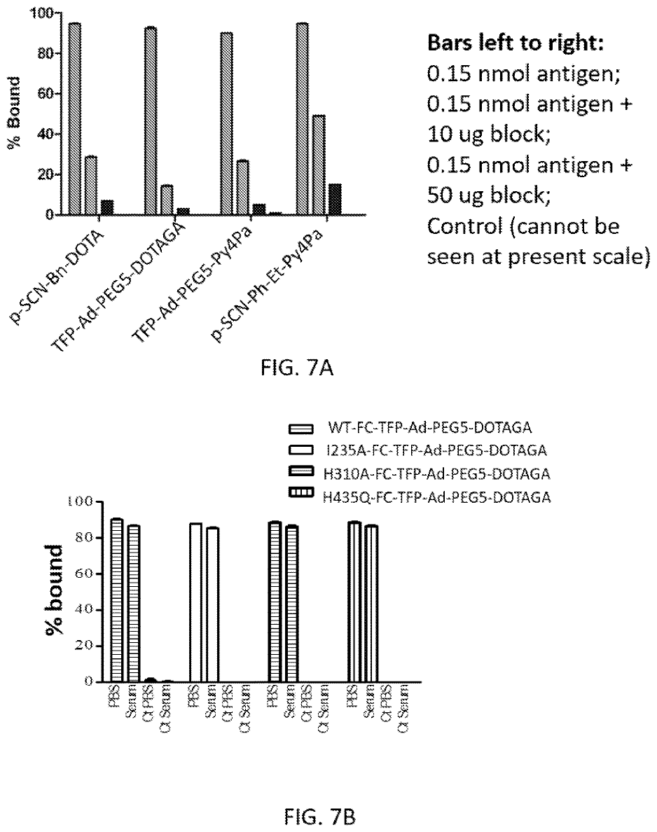

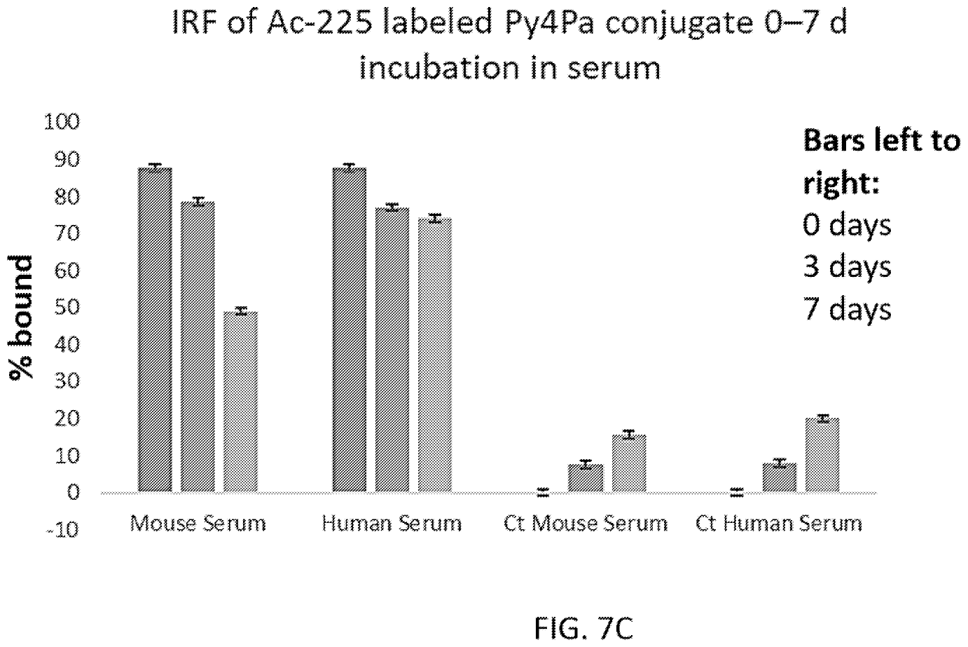

As shown in FIG. 7A for DLL3 all linker chelator combinations showed a similar immunoreactive fraction indicating no bias in labeling based upon the specific linker chelator combination, FIG. 7B shows that there was no effect due to Fc region mutations in immunoreactive fraction after 24 hours in PBS or serum, and FIG. 7C shows the immunoreactive fraction of 225AC labeled anti-DLL3 VHH-Fc (D102) and stability in serum and plasma.

### Example 24. Biodistribution of VHH-Fc Radioimmunoconjugates

### Biodistribution and Tissue Accumulation Over Time in HER2+BT474 Tumors

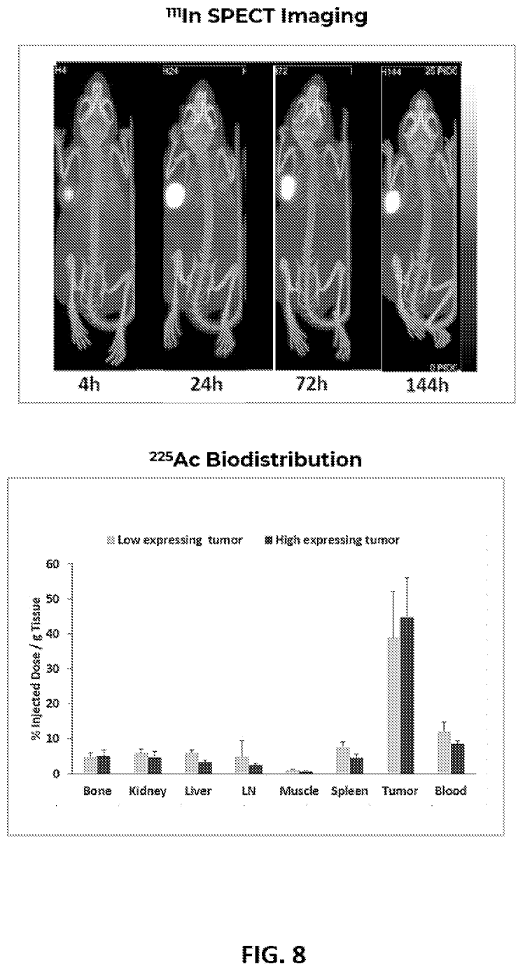

Imaging (e.g., using Indium-111 (111In)) provides for the ability to collect pharmacokinetic and biodistribution data that can be used to perform dosimetry calculations for treatment planning. (See, e.g., Sgouros G, Hobbs RF. “Dosimetry for radiopharmaceutical therapy.” Semin Nucl Med. 2014 May; 44(3):172-8).). Without being bound by theory, a quantitative demonstration of targeting observed with an imaging label is indicative of the ability to target with a radiolabel (e.g., an alpha emitter) capable of causing targeted cell death. Such phenomena is illustrated by FIG. 8, which illustrates that mice labeled with the imaging isotope 111In (top), exhibit accumulation of the therapeutic isotope 225Ac in tumors that express low amounts of antigen and high amounts of antigen, in this example DLL3 expressing SHP77 tumors and HER2 expressing BT474 tumors respectively.

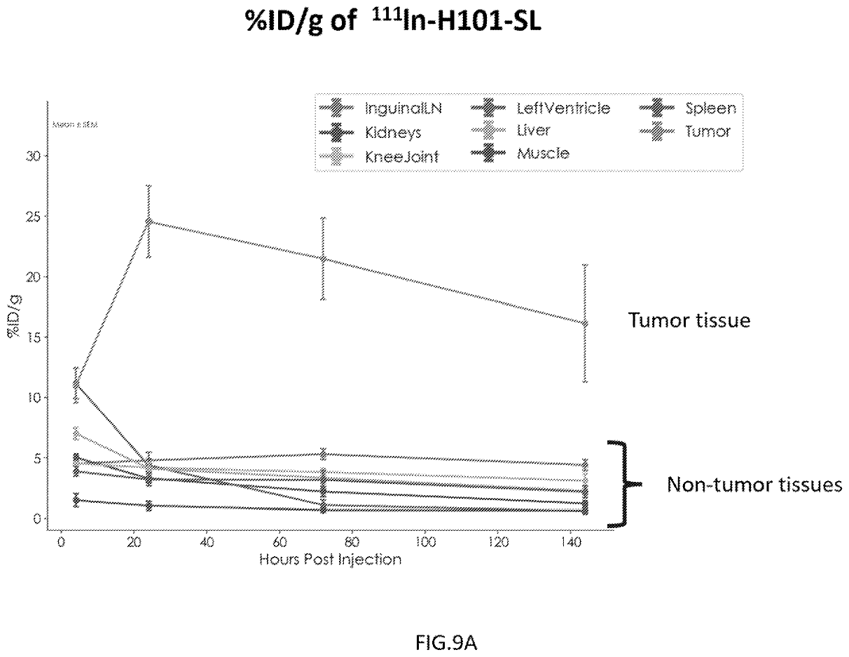

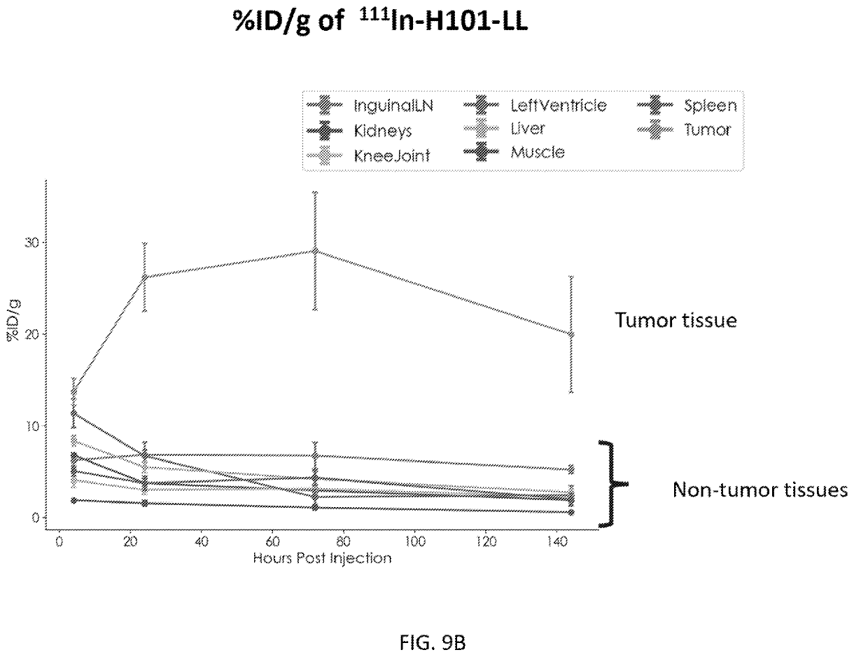

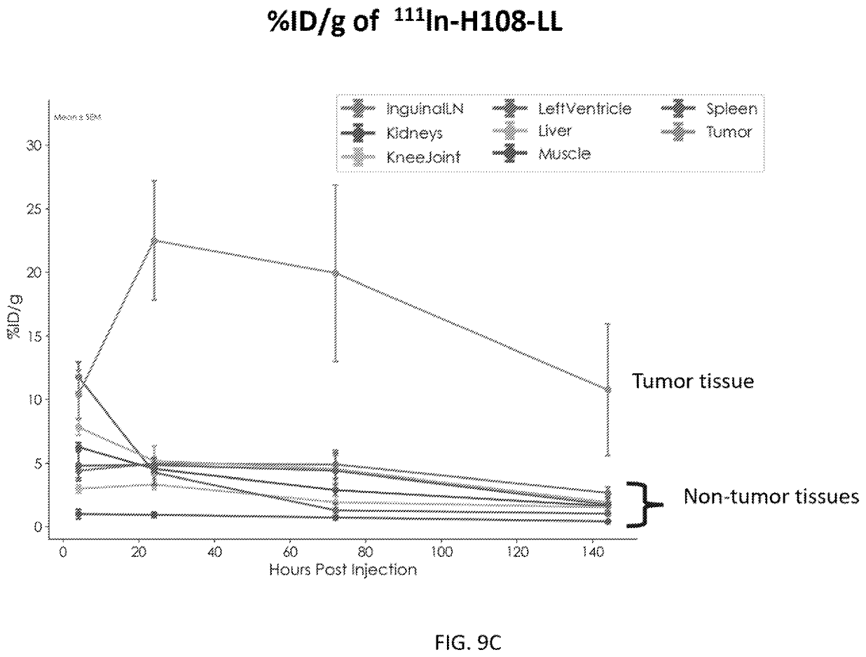

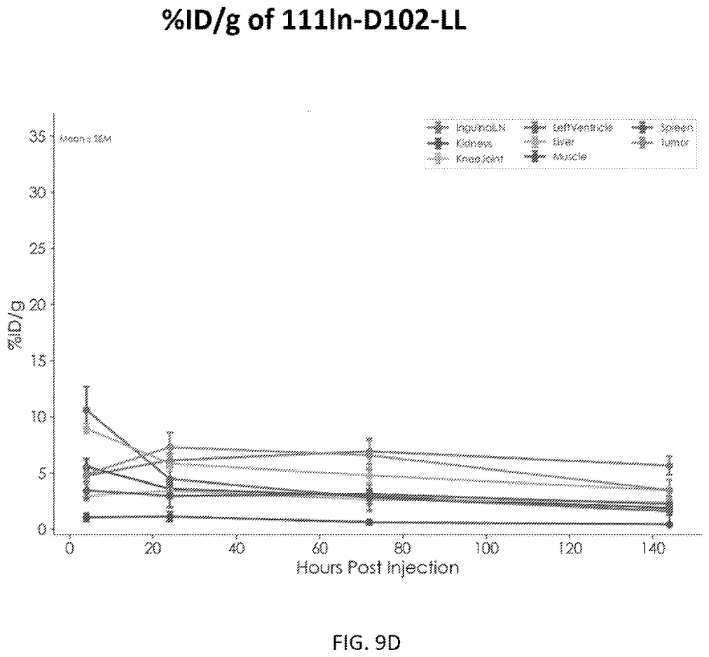

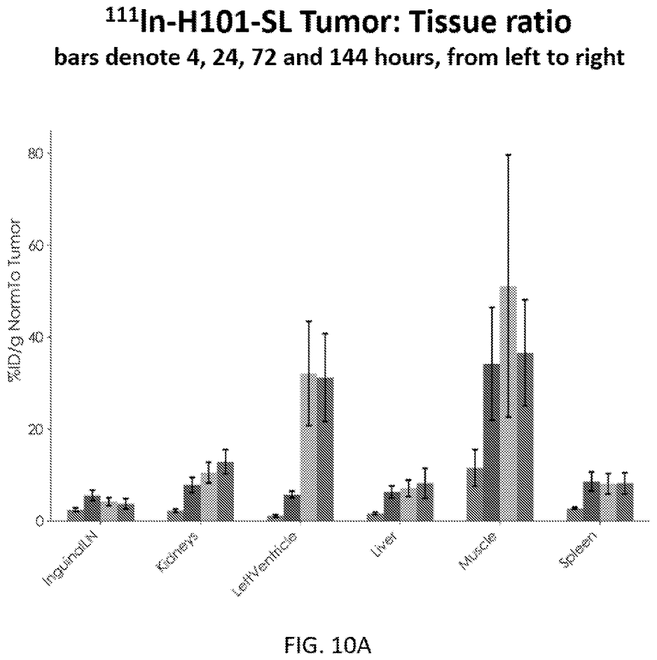

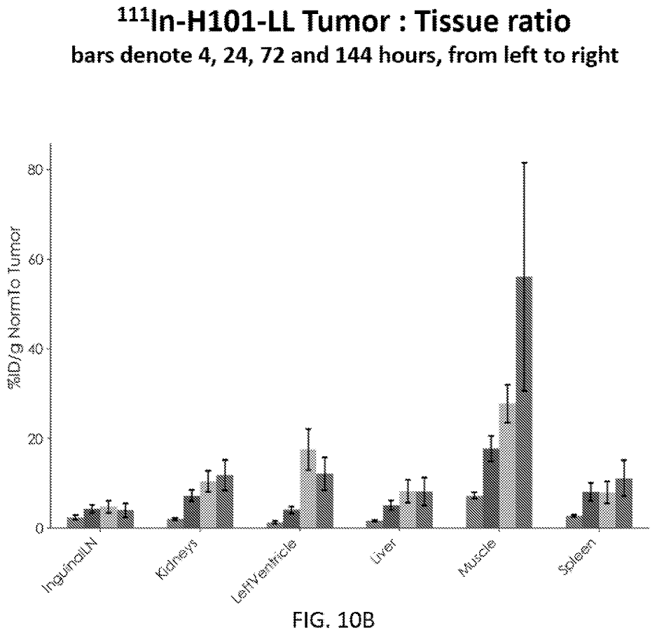

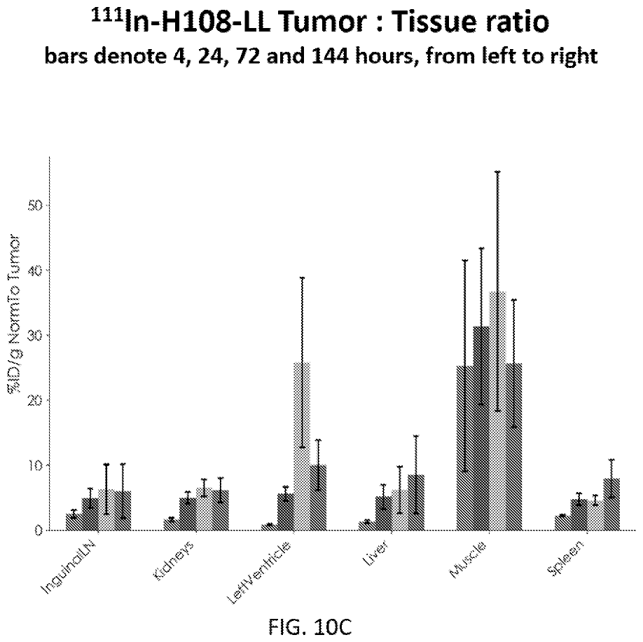

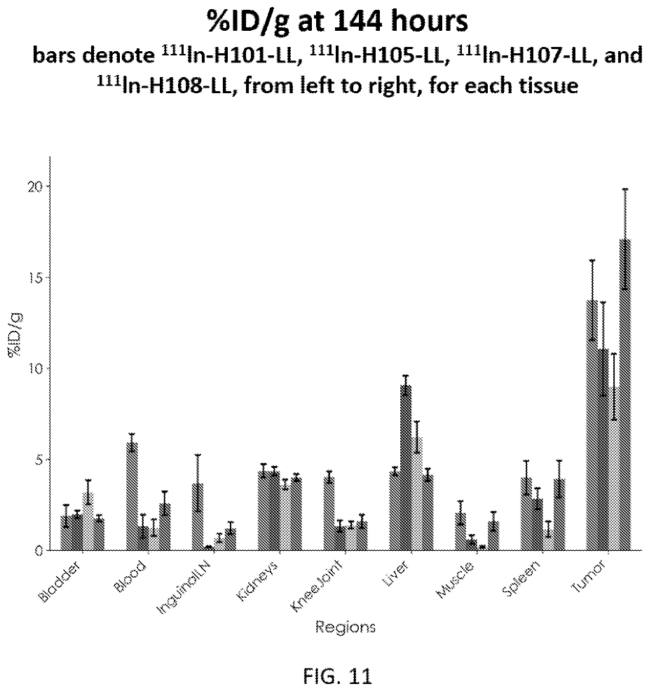

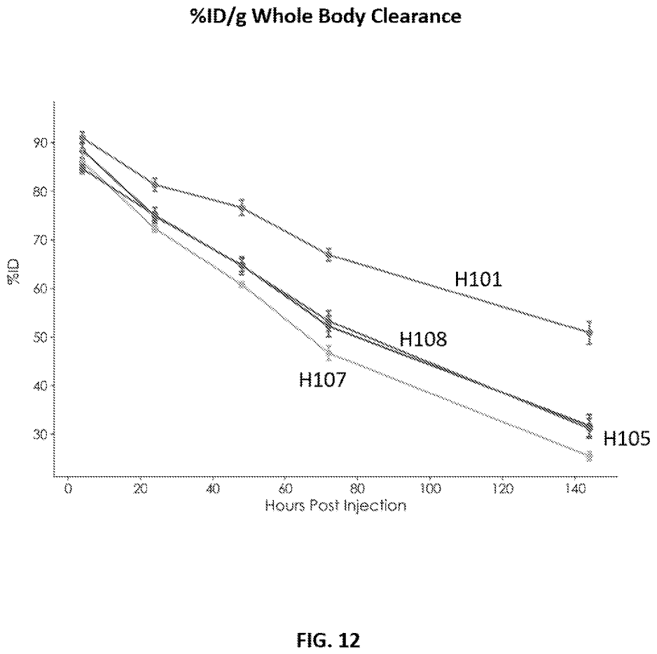

The objective of this study was to observe the biodistribution of 111In radiolabeled SPECT/CT imaging across select test articles in BT-474 tumor (breast cancer cells) bearing nude mice. The following articles were tested at a CAR of about 4: 111In-H101-short DOTA linker (p-SCN-Bn-DOTA, SL), 111In-H101-long DOTA linker (TFP-Ad-PEG5-DOTAGA, LL), 111In-H105-LL, 111In-H107-LL, and 111In-H108-LL. FIGS. 9A, 9B, and 9C show tissue accumulation over time for 111In-H101-SL, 111In-H101-LL, and 111In-H108-LL. FIG. 9D shows minimal tumor accumulation with DLL3 targeting VHH-Fc in HER2+ tumor model, further demonstrating specificity of the HER2 targeting VHH-Fcs. FIGS. 10A, 10B, and 10C show tumor:tissue ratios. In each case, the tumor:tissue ratios were greater than 5, indicating increased tumor accumulation and better profiles used for determining safety (e.g., as compared lower tumor:tissue ratios). FIG. 11 shows % ID/g at 144 hours for 111In-H101-LL, 111In-H105-LL, 111In-H107-LL, and 111In-H108-LL. In each case, the VHH-Fc variants show advantageous targeting of tumor tissue. FIG. 12 shows whole body clearance of VHH-Fc (H101) and VHH-Fc variants (H105, H107, and H108), wherein the VHH-Fc variants show increased clearance which can further be advantageous when considering safety and preventing unwanted tissue toxicity. In all cases, all test articles avoided significant kidney accumulation, further demonstrating favorable profiles for safety and avoiding unwanted tissue toxicity. Table 13 specifically shows the tumor accumulation for 111In-H101-LL, 111In-H105-LL, 111In-H107-LL, and 111In-H108-LL over time.

TABLE 13
Tumor accumulation of anti-HER2 VHH-Fc
variants (mean % ID/g; n = 4)

| | | 4 h | 24 h | 48 h | 72 h | 144 h |
|---|---|---|---|---|---|---|
| ¹¹¹In-H108-LL | mean | 4.7 | 12.2 | 14.4 | 12.7 | 13.7 |
| | SEM | 0.6 | 1.8 | 2.1 | 0.9 | 2.2 |
| ¹¹¹In-H101-LL | mean | 4.9 | 9.3 | 14.2 | 14.1 | 11.1 |
| | SEM | 0.5 | 1.1 | 2.0 | 2.6 | 2.6 |
| ¹¹¹In-H105-LL | mean | 4.9 | 7.1 | 9.0 | 9.4 | 9.0 |
| | SEM | 1.1 | 2.0 | 1.9 | 2.2 | 1.8 |
| ¹¹¹In-H107-LL | mean | 6.2 | 12.6 | 18.6 | 18.0 | 17.1 |
| | SEM | 1.1 | 1.9 | 2.3 | 2.6 | 2.8 |

Biodistribution and Tissue Accumulation Over Time in DLL3+SHP-77 Tumors

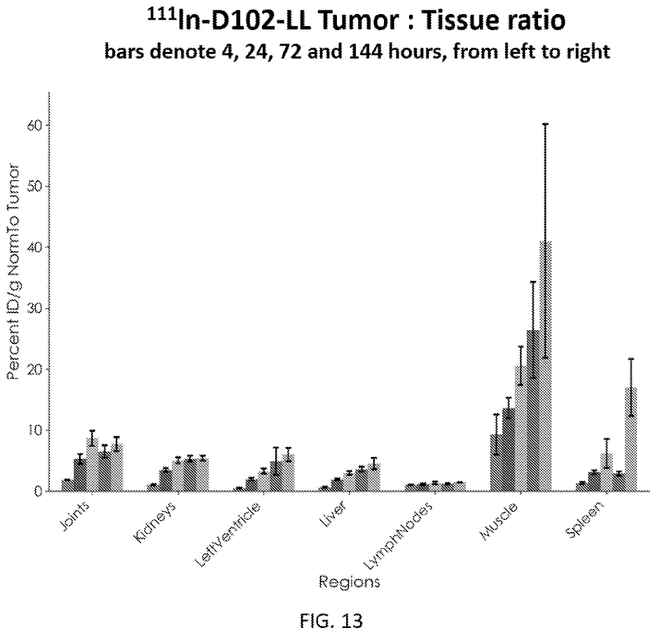

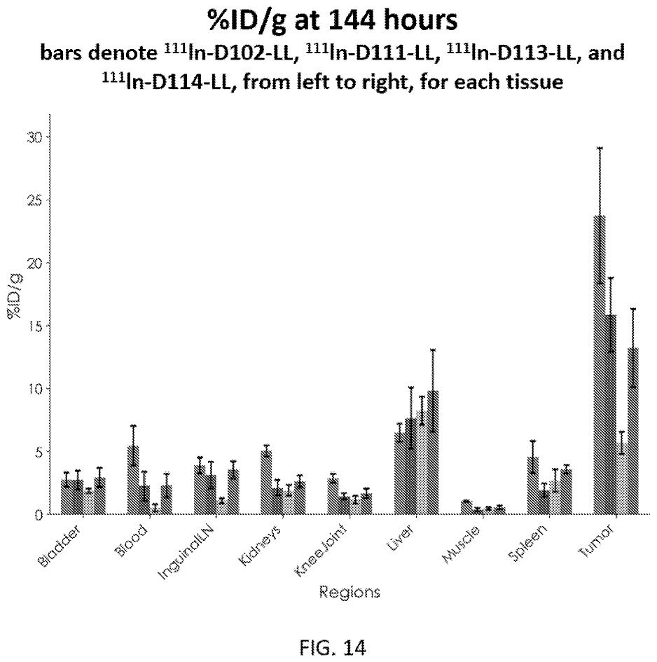

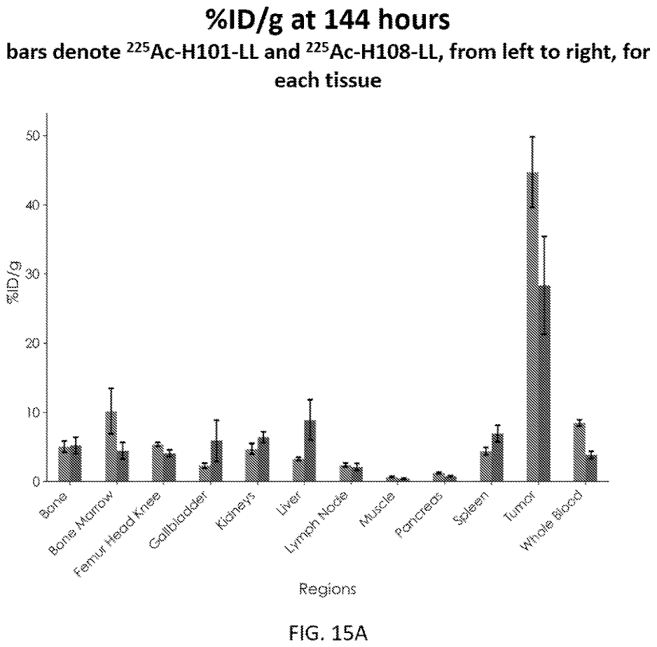

The objective of this study was to observe the biodistribution of 111In SPECT/CT across select test articles in SHP-77 tumor bearing nude mice. In contrast to HER2, DLL3 is generally present at lower copy numbers on the cell surface. Accordingly, the DLL3 represents the ability to target low copy number target proteins, whereas HER2 represents the ability to safely and effectively target high copy number target proteins. The following articles were tested: 111In-D102-long DOTA linker (LL), 111In-D111-LL, 111In-D113-LL, and 111In-D114-LL. Interestingly, similar targeting profiles and observations to the HER2 model were observed for the DLL3 model, demonstrating the ability to target high and low copy number targets. FIG. 13 shows 111In-D102-LL Tumor: Tissue ratios and FIG. 14 shows % ID/g at 144 hours for 111In-D102-LL, 111In-D111-LL, 111In-D113-LL, and 111In-D114-LL. As observed for HER2, anti-DLL3 VHH-Fc variants showed advantageous targeting of tumor tissue. Additionally, liver accumulation is indicative of increased clearance, which can further be advantageous when considering safety and preventing unwanted tissue toxicity. In all cases, all test articles avoided significant kidney accumulation, further demonstrating favorable profiles for safety and avoiding unwanted tissue toxicity. Table 14 specifically shows the tumor accumulation for 111In-D102-LL, 111In-D 111-LL, 111In-D113-LL, and 111In-D 114-LL over time.

TABLE 14
Tumor accumulation of anti-DLL3 VHH-Fc
variants (mean % ID/g; n = 4)

| | | 4 h | 24 h | 48 h | 72 h | 144 h |
|---|---|---|---|---|---|---|
| ¹¹¹In-D102-LL | mean | 6.0 | 12.8 | 18.0 | 19.0 | 23.7 |
| | SEM | 0.7 | 1.7 | 2.1 | 2.1 | 5.4 |
| ¹¹¹In-D111-LL | mean | 5.5 | 12.8 | 16.6 | 16.8 | 15.9 |
| | SEM | 1.4 | 1.1 | 2.0 | 2.3 | 2.9 |
| ¹¹¹In-D113-LL | mean | 4.5 | 8.7 | 10.0 | 9.4 | 5.7 |
| | SEM | 0.6 | 1.2 | 1.4 | 1.2 | 0.9 |
| ¹¹¹In-D114-LL | mean | 5.1 | 10.9 | 14.6 | 15.8 | 13.2 |
| | SEM | 0.5 | 0.9 | 1.6 | 2.4 | 3.1 |

Taken together, the 111In imaging results show that targeting of both high copy number and low copy number targets can be achieved with the radiolabeled VHH-Fcs and VHH-Fc variants. These results further indicate favorable safety and specificity profiles for targeting tumor tissue, avoiding non-tumor tissue, and in certain instances, effectively clearing radiolabeled VHH-Fcs (e.g., VHH-Fcs having mutations that reduced FcRn affinity).

### Biodistribution and Tissue Accumulation of Ac-225 Radiolabeled VHH-Fcs

The objective of this study was to observe biodistribution of (i) Ac-225 radiolabeled HER2 VHH-Fcs in a BT-474 tumor mouse model, as described above, and (ii) Ac-225 radiolabeled DLL3 VHH-Fcs in a SHP-77 tumor mouse model, as described above. Ex vivo radioactive quantitation in tumor and normal tissues was achieved by gamma counting.

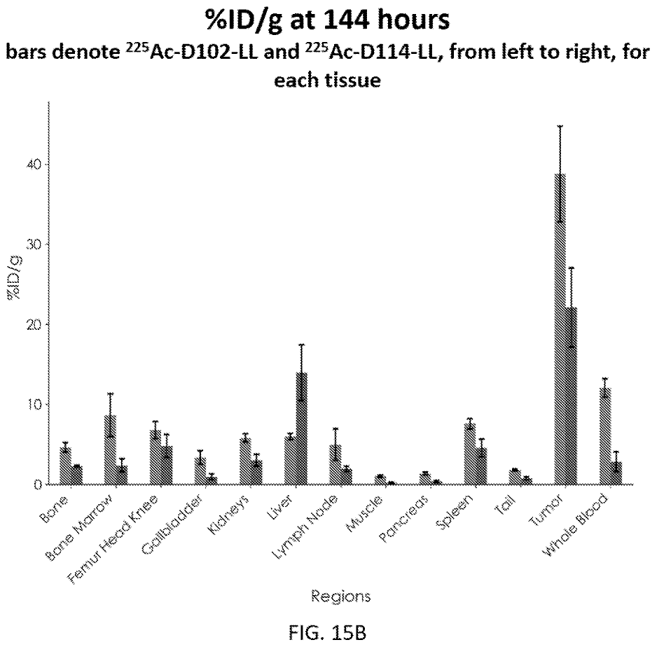

As described herein, the HER2 model represents a target with high receptor density on cancer cells (e.g., ˜300,000 copies/cell). FIG. 15A shows % ID/g at 144 hours for 225Ac-H101-LL and 225Ac-H108-LL. Both test articles showed advantageous targeting profiles, consistent with the 111In imaging data. Notably, specific targeting of tumor tissue was achieved with a favorable tumor:tissue ratio consistent with the imaging data. For the VHH-Fc variant 225Ac-H108-LL, lower radioactivity was detected in blood indicating more rapid clearance of the VHH-Fc variant (consistent with results in Example 10). 225Ac-H108-LL also demonstrated lesser kidney accumulation and greater liver accumulation indicating increased clearance through the hepatic route and avoidance of the kidneys which further supports an increase in the safety profile of VHH-Fcs with FcRn mutations. The lower tumor accumulation for 225Ac-H108-LL can be attributed to the decreased serum half-life (i.e., more rapid clearance). Table 15 further shows tumor volume through Day 6 post injection, wherein tumor volumes decreased after administration of 225Ac-H101-LL and 225Ac-H108-LL. Table 15 indicates that mice injected with VHH immunoconjugates with wild-type Fc or with FcRn mutations both saw tumor shrinkage by 6 days post injection.

TABLE 15
Tumor volumes before and after anti-HER2
VHH-Fc treatment (mean mm³; n = 5)

| | | | | | | | | 0 | | |
|---|---|---|---|---|---|---|---|---|---|---|
| | Day | −15 | −11 | −8 | −6 | −4 | −1 | (dose) | 3 | 6 |
| ²²⁵AC- | mean | 57.4 | 66.5 | 51.9 | 54.7 | 65.6 | 73.9 | 74.4 | 31.3 | 47.0 |
| H101- | | | | | | | | | | |
| LL | SD | 19 | 10 | 10 | 10 | 20 | 36 | 22 | 11 | 12 |
| ²²⁵AC- | mean | 46.4 | 56.5 | 67.9 | 63.2 | 67.3 | 62.6 | 78.1 | 46.2 | 51.2 |
| H108- | | | | | | | | | | |
| LL | SD | 9 | 11 | 16 | 12 | 14 | 14 | 27 | 19 | 23 |

As also described herein, DLL3 represents a target with low target density on cancer cells (e.g., ˜3,000 copies/cell). FIG. 15B shows % ID/g at 144 hours for 225Ac-D102-LL and 225Ac-D114-LL. Both test articles showed advantageous targeting profiles, consistent with the 111Ln imaging data. Additionally, specific targeting of tumor tissue was achieved with a favorable tumor:tissue ratio consistent with the imaging data. As observed with the anti-HER2 VHH-Fc variants, for the VHH-Fc variant 225Ac-D114-LL, the VHH-Fc variants show increased clearance and decreased kidney exposure which can further be advantageous when considering safety and preventing unwanted tissue toxicity. The lower tumor accumulation for 225Ac-D114-LL can be attributed to the decreased serum half-life (i.e., more rapid clearance).

### Example 25. Low Toxicity Associated with VHH-Fc Radioimmunoconjugates

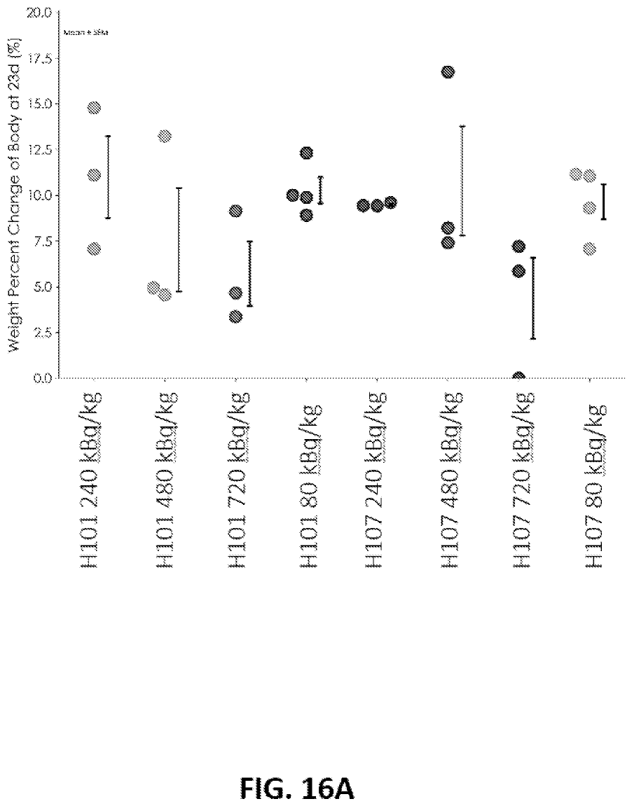

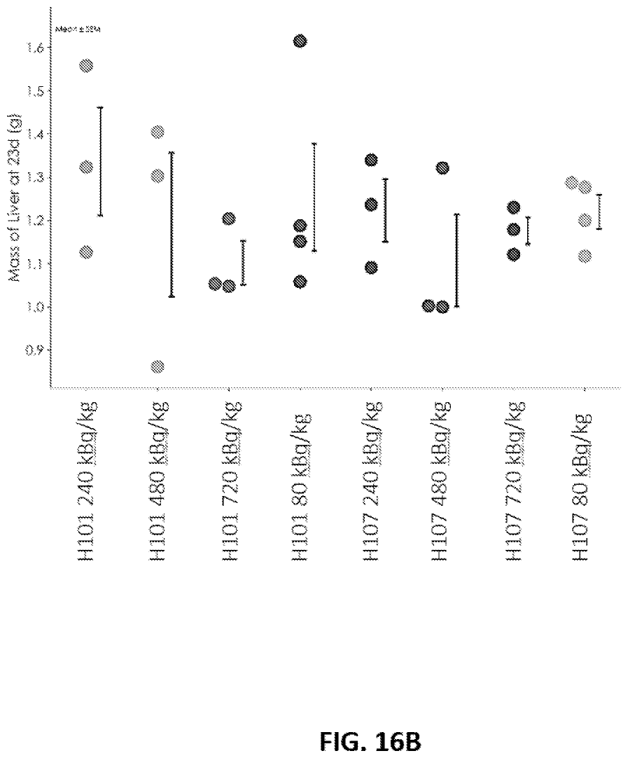

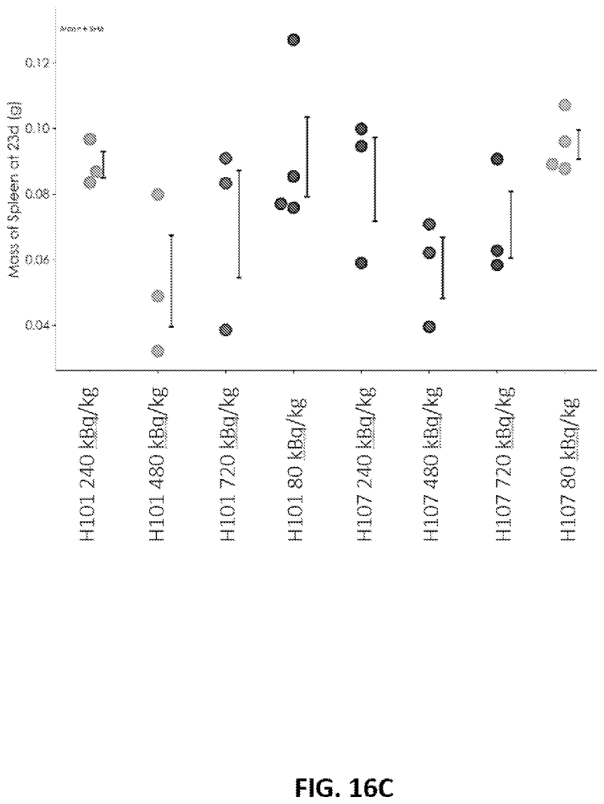

A study was undertaken to determine the tolerability of VHH-Fc loaded with 225AC. Naïve female athymic nude mice were injected intravenously (IV) into the tail vein with 225Ac-H101-447804 (anti-HER2 with wildtype Fc, TFP-Ad-PEG5-DOTAGA) or 225Ac-H107-447804 (anti-HER2 with H310A Fc, TFP-Ad-PEG5-DOTAGA) at four different activity dose levels (18.5 kBq, 12 kBq, 6 kBq, 2 kBq). Activity dose volume was adjusted for body weights measured on the injection day. All animals were monitored for adverse effects daily. Body weights were recorded three (occasionally two or four) times a week for all animals until end of study at 23 days post-injection. 23 Days post-injection all animals were sacrificed. Carcasses underwent necropsy. Whole body, spleen, and liver weights were recorded. FIGS. 16A, 16B, and 16C show that, as measured by percent weight change (16A), liver mass (16B), and spleen mass (16C) All doses of 225Ac-labeled antibodies of up to 740 kBq/kg were well tolerated and no indications of radiation sickness were observed.

### Example 26. Radiolabeling with Lu-177

50 μg of test article (D102) was diluted to 100 μL with 0.1 M ammonium acetate buffer pH 5.5 in a 500 μL lo-bind Eppendorf tube and 51 MBq in 3.2 μL-3.5 μL of 177-Lutetium chloride was added and mixed with a pipette. The reaction mixtures were incubated at 37° C. in an incubator for 3 hours and samples taken at 30 min, and 1, 2, and 3 h for iTLC analysis. Results of the labeling are shown in Table 16 below, and indicate efficient labeling with 177-Lutetium.

TABLE 16

| | |
|---|---|
| Test article | Incubation time at 37 deg C. |

| D102 | 30 min | 60 min | 2 hr | 3 hr |
|---|---|---|---|---|
| TFP-Ad-PEG5- | 99.0% | 99.2% | 99.2% | 99.3% |
| DOTAGA | | | | |

After dilution in PBS/ascorbate and storage at 4° C. the purity as assessed by iTLC analysis as in Example 22.

To analyze stability, 50 μL of test article was added to 200 μL of PBS/ascorbate and stored at 4° C. The samples were analyzed by iTLC and SEC-HPLC after 1-4 h and 18-24 h. Results are shown in Table 17 below, and indicate stability of the construct.

TABLE 17

| | |
|---|---|
| Test article | Incubation time |

| D102 | 1 hr | 1 d |
|---|---|---|
| TFP-Ad-PEG5- | 98.8% | 98.6% |
| DOTAGA | | |

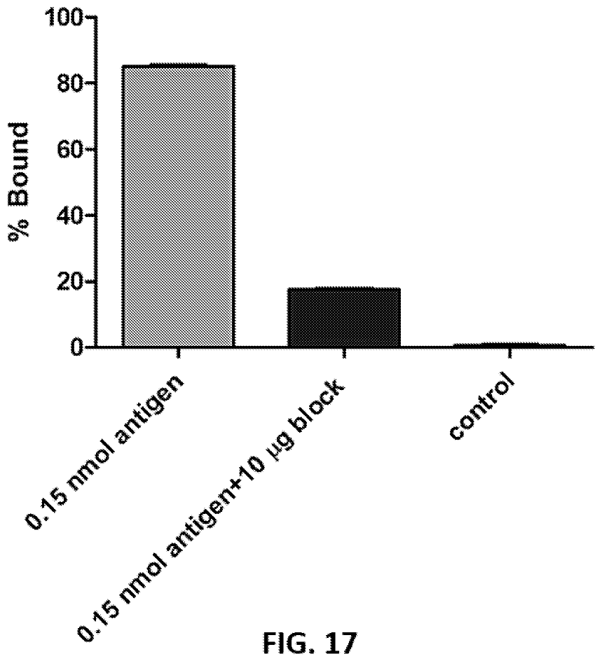

The Lu-177 conjugate was analyzed by the IRF assay described above in Example 23 and the results are shown in FIG. 17. In this example, the control is beads with no antigen loaded.

### Example 27. Generation and Characterization of DLL3 Binding Regions

Camelids (llamas and alpacas) were immunized subcutaneously (SC) with recombinant human DLL3 in complete Freud's adjuvant (CFA) or incomplete Freud's adjuvant (IFA) at 2-4 week intervals. Serum titer response was assessed using dilution series of serum. Sera samples were incubated with multiplexed beads differentially optically encoded to various DLL3 antigens (human, mouse, cynomolgus monkey). Binding of antigen-specific antibodies in the serum to the beads was then detected using a fluorescently labelled secondary antibody via high-throughput, plate-based flow cytometry. Samples were selected for screening and peripheral blood mononuclear cells were collected.

### Single-Cell Screening and Recovery

Peripheral blood mononuclear cells were thawed, activated in culture to generate memory B cells, and enriched for heavy chain-only antibody-secreting B cells before screening. Single B cells secreting target-specific antibodies were identified and isolated using a multi-step assay assessing both internalization in cells and binding to DLL3 immobilized on beads. Cross-reactivity to species homologs was assessed using a multiplexed bead assay using differentially optically encoded beads, each conjugated to different species of DLL3 antigens (human, mouse, cynomolgus monkey) and binding was detected using a fluorescently labelled secondary antibody specific to alpaca IgG subclasses 2 and 3. Internalization was assessed by flowing in HEK293T cells expressing human DLL3 and internalizing antibodies were detected using a pH-sensitive fluorescent reagent.

### Single-Cell Sequencing and Bioinformatic Analysis

Single-cell polymerase chain reaction (PCR) and custom molecular biology protocols generated NGS sequencing libraries (MiSeq, Illumina) using automated workstations (Bravo, Agilent). Sequencing data were analyzed using a custom bioinformatics pipeline to yield heavy chain sequences for recovered antibody-secreting cells. Each sequence was annotated with the closest germline (V(D)J) genes and degree of somatic hypermutation. Antibodies were considered members of the same clonal family if they shared the same inferred heavy V and J genes and had the same CDR3 length.

VHH-Fc plasmids were generated by cloning the VHH sequence, with a hinge and Fc portion (human IgG1 CH2-CH3) into a mammalian expression vector. In some instances, mutations were introduced into the Fc portion. To produce recombinant VHH-Fc and variants thereof, plasmid was transfected into HEK293.SUS cells (ATUM, or similar). After 3-5 days of secretion, the antibody-containing supernatant was cleared of cells by centrifugation and sterile filtration. Antibodies were purified using Mab Select SuRe PCC column (GE, Cat #: 11003495) and buffer exchanged into PBS, pH 7.0. Proteins were quantified using A280 or BCA. The purity of the antibodies were tested by SDS-PAGE, capillary electrophoresis, HPLC-SEC and LC-MS using standard protocols.

A total of 209 VHH clones were tested for properties important for the development of immunoconjugates useful for the delivery of toxic payloads. These criteria included binding to murine DLL3, binding to cynoDLL3, binding to human DLL3, the ability to be internalized by target expressing cells, the absence of lysine residues in CDR regions, high sequence redundancy and absence of known sequence liabilities for developability.

Of these 209 clones, 46 were selected for expression and purification as VHH-Fc (with wildtype Fc and modified hinge region, SEQ ID: 42) for further analysis. A summary of the data generated on these 46 VHH.Fcs is shown in Tables 18 to 20.

### High-Throughput Antibody Expression and Purification

The variable [V(D)J] region of each antibody heavy chain was synthesized and inserted into expression plasmids. Plasmids were verified by Sanger sequencing. Chimeric human Fc, camelid VHH (VHH-Fc) antibodies were recombinantly produced by transient transfection. Antibody-encoding plasmid DNA was transfected into Expi293F cells (Thermo Fisher Scientific). Antibody titers were measured by biolayer interferometry on an Octet HTX instrument (ForteBio). Antibodies were purified using protein A-based purification and quantified by UV/Vis Spectroscopy at 280 nm absorbance.

### Antibody Bead Binding and Cell Internalization Validation

Recombinant VHH-Fc antibodies were confirmed to bind targets and induce internalization via high-throughput flow cytometry using fluorescently labelled anti-human IgG. In a multiplexed bead-based assay, optically encoded beads were conjugated to one of the following antigens: human DLL3, mouse DLL3, or cynomolgus monkey DLL3. Purified VHH-Fc antibodies were incubated with target-conjugated beads at 25 nM antibody concentration for 30 minutes at 4° C. Beads were washed and binding was detected using a fluorescently labelled secondary antibody. In a live cell-based internalization assay, purified VHH-Fc antibodies were incubated with HEK293T cells expressing human DLL3, parental HEK293T cells or SHP-77 cells at 5 nM antibody concentration and a pH-sensitive fluorescent reagent for two hours at 37° C. Fluorescence was measured using high-throughput, plate-based flow cytometry. An irrelevant antibody, chimeric human Fc camelid VHH specific to HER2 (VHH-Fc anti-HER2) was used as a negative control. Median fluorescence intensity of each antibody was normalized over median fluorescence intensity of the negative control.

### SPR Binding Experiments

All SPR binding experiments were performed on a Carterra LSA instrument equipped with an HC-30M chip type (Carterra-bio) using a 384-ligand array format as described herein. The HC-30M chip was prepared by immobilizing a goat anti-human IgG Fc antibody (Southern Biotech #2014-01) via direct coupling: The chip surface was first activated by flowing a freshly prepared 1:1:1 activation mix of 100 mM MES (pH 5.5), 100 mM sulfo-N-hydroxysuccinimide, and 400 mM 1-ethyl-3-(3-dimethylaminopropyl)carbodiimide for 7 minutes, and goat anti-human IgG Fc antibody diluted to 50 ug/ml in 10 mM NaOAc (pH 4.25) buffer+0.01% Tween was injected onto the chip surface for 10 minutes. The chip surface was quenched by flowing 1 M ethanolamine for 7 minutes, followed by two wash steps of 15 seconds each in 25 mM MES (pH 5.5) buffer. The test antibodies diluted to 5 ug/ml in HEPES-buffered saline containing 0.05% Tween 20 and 3 mM EDTA (HBSTE)+0.1% BSA running buffer were captured on the chip surface for 5 minutes. Relevant benchmarks and negative control antibodies (VHH-Fc anti-HER2, VHH-Fc anti-DLL3, Rovalpituzumab) were also captured on the chip surface.

For binding kinetics and affinity measurements, a threefold dilution series of the antigen of interest (human DLL3), starting at 300 nM in HEPES-buffered saline containing 0.05% Tween 20 and 3 mM EDTA (HBSTE)+0.1% BSA running buffer, was sequentially injected onto the chip surface. For each concentration, the antigen was injected for 10 min (association phase), followed by running buffer injection for 15 min (dissociation phase). The data were analyzed using the Carterra Kinetics analysis software using a 1:1 Langmuir binding model to determine apparent association (ka) and dissociation (kd) kinetic rate constants and binding affinity constants (Kd).

### Capillary Electrophoresis Sodium Dodecyl Sulfate (CE-SDS)

The purity of the expressed and purified VHH-Fc antibodies was analyzed by denaturing CE-SDS using the LabChip GXII Touch instrument (Perkin Elmer, Protein Express LabChip #760528) according to the manufacturer's protocol. Two (2) uL of VHH-Fc solution at a concentration of 0.35 mg/mL in PBS was mixed with a non-reducing denaturing buffer solution (Perkin Elmer Reagent Kit #CLS960008) and incubated at 70° C. for 10 min. Separation was performed using the HT Antibody Analysis 200 assay setting on the LabChipGXII Touch instrument (Perkin Elmer). The data was analyzed using the LabChip GX Reviewer Software (Perkin Elmer).

### Dynamic Light Scattering (DLS)

Percent aggregation and polydispersity of VHH-Fc antibodies was assessed by DLS on a DynaPro® Plate Reader III instrument (Wyatt Technology). Seven (7) μL of each sample at 0.35 mg/mL in PBS were dispensed into glass-bottom 1536 well Sensoplates (Greiner Bio-One, #783892) and covered with silicon oil. DLS of individual samples was then acquired at 20° C. with 5×5 seconds acquisitions per sample. Data was analyzed in the Dynamics software (Wyatt Technology, v 7.10.1.21) using the regularization algorithm and replicate measurements with less than 60% of acquisitions unmarked were omitted from the analysis. Replicates were then averaged using an in-house developed Python script. Filter settings were a maximum sum of squares deviation of 50 between autocorrelation function and data fit, a minimum and maximum autocorrelation function amplitude of 0.05 and 1, respectively, and a baseline limit of 1±0.005. Percent polydispersity and percent mass of soluble mAbs were calculated for the size range of 1-10 nm.

Nanoscale Differential Scanning Fluorimetry (nDSF)

All nano-DSF studies were performed using the Nanotemper Prometheus NT.Plex instrument equipped with a Backreflection Optics and an NT.Robotic Autosampler for automated sample loading and measurement.

Samples at 350 μg/mL were loaded by capillarity into premium grade nDSF capillaries (NanoTemper, Cat #PR-AC006). Capillaries were then placed on the Prometheus thermal element and subjected to a temperature ramping of 1° C./minute from 20° C. to 95° C. The melting point (Tm, in ° C.) was obtained by monitoring the intrinsic tryptophan and tyrosine fluorescence at the emission wavelengths of 330 nm and 350 nm. To generate an unfolding curve, the ratio of the fluorescence intensities (F350 nm/F330 nm) was plotted versus the temperature. The Tm corresponds to the inflection point of the unfolding curve and was determined via the derivative of the curve using the NanoTemper PR.Stability Analysis software (version 1.1). The onset of aggregation (Tagg, in ° C.) was obtained by monitoring the light backreflection of protein aggregates and determined using the NanoTemper's PR.Stability Analysis software (version 1.1).

Analytical Size-Exclusion Chromatography (aSEC)

The relative percentage of monomer, high molecular weight (HMW), and low molecular weight (LMW) species in purified VHH-Fc antibodies was assessed using aSEC. Using a Vanquish Duo UHPLC System for Dual LC (Thermo Fisher Scientific), 5 μL of each sample at 0.35 mg/mL was injected onto a size exclusion column (ACQUITY UPLC Protein BEH SEC Column, 200 Å, 1.7 μm, 4.6 mm×150 mm, Waters #186005226). The mobile phase (100 mM sodium phosphate pH 6.8, 250 mM NaCl; Fisher Scientific #S468-500, #S373-500, and #S271-500) was applied to the column for 10 minutes per injection at a flow rate of 0.3 mL/min to separate species based on their size. Chromatograms monitoring absorbance at 280 nm were acquired and analyzed using Chromeleon software (Thermo Fisher Scientific, v7.3). The relative percentage of each species was determined based on the integrated area of each peak.

Analytical Hydrophobic Interaction Chromatography (aHIC)

Relative surface hydrophobicity of the purified VHH-Fc antibodies was assessed by aHIC. Using a Vanquish Duo UHPLC System for Dual LC (Thermo Fisher Scientific), 5 μL of each sample at 0.35 mg/mL was injected onto a hydrophobic interaction column (TSKgel Butyl-NPR, 2.5 μm, 4.6 mm ID×3.5 cm, TOSOH #0014947). A linear gradient method from 42% to 0% buffer A over 6 minutes with a flow rate of 0.5 mL/min was used to separate samples based on their surface hydrophobicity properties (buffer A: 25 mM sodium phosphate pH 7.0, 2.5 M ammonium sulfate; buffer B: 25 mM sodium phosphate pH 7.0; Fisher Scientific #S468-500, #S373-500, and #A702-3). Chromatograms monitoring absorbance at 280 nm were acquired and analyzed using Chromeleon software (Thermo Fisher Scientific, v7.3). Relative hydrophobicity of each sample was determined based on retention time of the largest peak by integrated area.

TABLE 18
Summary of initial screening VHH formatted on
Wildtype Fc/modified hinge (SEQ ID NO: 42)

| Clone | hDLL3 | mDLL3 | cDLL3 | Internalisation | | [Liability] | CDR |
|---|---|---|---|---|---|---|---|
| ID | Binding | binding | Binding | HEK-DLL3 | Redundancy | Status | liability |
| 4 | Yes | Yes | Yes | Yes | 3 | 1 | 0 |
| 5 | Yes | Yes | Yes | Yes | 22 | 2 | 0 |
| 6 | Yes | Yes | Yes | Yes | 4 | 2 | 2 |
| 22 | Yes | Yes | Yes | Yes | 1 | 2 | 2 |
| 24 | Yes | Yes | Yes | Yes | 24 | 1 | 0 |
| 28 | Yes | Yes | Yes | Yes | 11 | 1 | 0 |
| 29 | Yes | Yes | Yes | Yes | 12 | 1 | 0 |
| 33 | Yes | Yes | Yes | Yes | 8 | 1 | 0 |
| 34 | Yes | Yes | Yes | | 2 | 1 | 0 |
| 39 | Yes | Yes | Yes | Yes | 4 | 2 | 0 |
| 40 | Yes | Yes | Yes | | 5 | 3 | 1 |
| 41 | Yes | Yes | Yes | Yes | 3 | 1 | 0 |
| 45 | Yes | Yes | Yes | Yes | 5 | 1 | 0 |
| 49 | Yes | Yes | Yes | Yes | 1 | 2 | 1 |
| 51 | Yes | Yes | Yes | Yes | 1 | 2 | 2 |
| 53 | Yes | Yes | Yes | Yes | 7 | 0 | 0 |
| 54 | Yes | Yes | Yes | Yes | 3 | 1 | 0 |
| 60 | Yes | Yes | Yes | Yes | 1 | 2 | 0 |
| 87 | Yes | Yes | Yes | Yes | 4 | 2 | 1 |
| 88 | Yes | Yes | Yes | Yes | 4 | 2 | 1 |
| 90 | Yes | Yes | Yes | Yes | 2 | 2 | 1 |
| 91 | Yes | Yes | Yes | Yes | 3 | 1 | 0 |
| 97 | Yes | Yes | Yes | Yes | 13 | 1 | 0 |
| 100 | Yes | Yes | Yes | Yes | 7 | 2 | 2 |
| 104 | Yes | Yes | Yes | Yes | 8 | 1 | 0 |
| 105 | Yes | Yes | Yes | Yes | 66 | 2 | 0 |
| 106 | Yes | Yes | Yes | Yes | 12 | 0 | 0 |
| 107 | Yes | Yes | Yes | | 11 | 2 | 1 |
| 109 | Yes | Yes | Yes | | 7 | 2 | 1 |
| 112 | Yes | Yes | Yes | Yes | 4 | 2 | 1 |
| 113 | Yes | Yes | Yes | | 2 | 2 | 0 |
| 118 | Yes | | Yes | Yes | 2 | 0 | 0 |
| 119 | Yes | Yes | Yes | Yes | 2 | 1 | 0 |
| 123 | Yes | Yes | Yes | Yes | 1 | 2 | 1 |
| 126 | Yes | | Yes | Yes | 14 | 2 | 1 |
| 130 | Yes | Yes | Yes | | 4 | 2 | 2 |
| 162 | Yes | Yes | Yes | Yes | 24 | 2 | 2 |
| 163 | Yes | | Yes | Yes | 18 | 2 | 1 |
| 166 | Yes | Yes | Yes | | 12 | 2 | 1 |
| 167 | Yes | | Yes | Yes | 11 | 2 | 1 |
| 168 | Yes | Yes | Yes | Yes | 1 | 2 | 1 |
| 179 | Yes | | Yes | Yes | 12 | 2 | 1 |
| 186 | Yes | | Yes | Yes | 2 | 2 | 0 |
| 187 | Yes | Yes | Yes | Yes | 4 | 2 | 1 |
| 189 | Yes | Yes | Yes | Yes | 2 | 2 | 0 |
| 191 | Yes | | Yes | Yes | 2 | 2 | 1 |

mDLL3 = mouse DLL3;
cDLL3 = cynoDLL3;
hDLL3 = human DLL3;
blank cells indicate no binding observed in initial screening assays

TABLE 19
Summary of initial screening, biophysical characteristics
VHH formatted on Wildtype Fc/modified hinge (SEQ ID NO: 42)

| | Purified | % Purity | | | Hydrodynamic | % | % |
|---|---|---|---|---|---|---|---|
| Clone | Titer | analytical | | | radius (nm) | Polydispersity | Aggregation |
| ID | (mg/L) | SEC | PI | PD | DLS | DLS | DLS |
| 4 | 256.71 | 100 | 1.9 | 2.1 | 5.77 | 35.50 | 97.76 |
| 5 | 294.03 | 100 | 5.2 | 4 | 5.18 | 36.62 | 97.35 |
| 6 | 260.96 | 100 | 4.1 | 4.5 | 5.86 | 41.76 | 98.85 |
| 22 | 1.79 | 0 | | | nan | nan | 0.00 |
| 24 | 272.8 | 100 | 2.1 | 2.5 | 4.80 | 27.34 | 97.40 |
| 28 | 279.45 | 100 | 1.9 | 1.8 | 5.32 | 30.27 | 97.42 |
| 29 | 199.76 | 100 | 2.5 | 2.3 | 4.73 | 24.27 | 97.97 |
| 33 | 119.52 | 98.38 | 2 | 1.8 | 5.56 | 31.19 | 97.38 |
| 34 | 277.31 | 100 | 2 | 2 | 4.94 | 23.29 | 93.74 |
| 39 | 270.79 | 100 | 5.1 | 10.3 | 4.86 | 26.35 | 98.94 |
| 40 | 265.95 | 100 | 2.6 | 4.2 | 4.44 | 18.93 | 99.55 |
| 41 | 229.8 | 100 | 2.8 | 2.1 | 5.28 | 30.13 | 96.07 |
| 45 | 305.46 | 100 | 1.2 | 1.5 | 5.84 | 39.01 | 97.81 |
| 49 | 182.91 | 94.79 | 4 | 5.9 | 5.32 | 30.54 | 45.44 |
| 51 | 238.05 | 100 | 1 | 1.2 | 4.51 | 19.91 | 99.98 |
| 53 | 288.51 | 100 | 1.9 | 2.5 | 5.05 | 34.15 | 98.00 |
| 54 | 281.75 | 100 | 1.9 | 2.9 | 5.07 | 28.53 | 98.36 |
| 60 | 273.37 | 100 | 1.1 | 1.2 | 5.58 | 25.80 | 98.47 |
| 87 | 202.48 | 98.71 | 1.1 | 1.4 | 5.31 | 34.60 | 99.27 |
| 88 | 137.34 | 99.34 | 4.8 | 2.9 | 4.05 | 24.06 | 91.57 |
| 90 | 284.07 | 100 | 3.6 | 2.3 | 4.86 | 25.24 | 98.64 |
| 91 | 303 | 100 | 3.5 | 3.1 | 5.57 | 30.95 | 96.90 |
| 97 | 289.44 | 100 | 1.6 | 1.3 | 5.24 | 28.87 | 99.31 |
| 100 | 228.26 | 100 | 1.6 | 1.3 | 4.87 | 29.93 | 96.76 |
| 104 | 268.28 | 100 | 3.9 | 2.4 | 6.28 | 23.39 | 90.13 |
| 105 | 1.08 | 0 | | | 8.87 | 14.17 | 18.60 |
| 106 | 254.7 | 95.29 | 1.1 | 1 | 6.74 | 29.62 | 99.94 |
| 107 | 282.08 | 100 | 1.6 | 2 | 5.22 | 31.31 | 98.30 |
| 109 | 289.91 | 100 | 1.1 | 1 | 7.76 | 32.73 | 46.68 |
| 112 | 311.61 | 100 | 2.4 | 2 | 5.33 | 34.08 | 98.32 |
| 113 | 157.14 | 98.65 | 9.2 | 5.8 | 5.38 | 35.88 | 98.01 |
| 118 | 16.42 | 0 | | | 8.17 | 10.84 | 18.66 |
| 119 | 267.47 | 100 | 3.2 | 2.2 | 4.41 | 14.48 | 99.95 |
| 123 | 247.72 | 100 | 1.4 | 1.1 | 5.53 | 33.23 | 97.09 |
| 126 | 281.1 | 100 | 1.3 | 1 | 5.47 | 35.42 | 98.92 |
| 130 | 274.63 | 100 | 1.6 | 1.1 | 4.44 | 19.82 | 98.74 |
| 162 | 307.01 | 100 | 2.4 | 2 | nan | nan | nan |
| 163 | 292.7 | 100 | 2.9 | 2.2 | 4.81 | 24.32 | 99.01 |
| 166 | 257.03 | 100 | 1.3 | 1.1 | 5.55 | 39.27 | 99.47 |
| 167 | 289.82 | 100 | 5.1 | 3.7 | 4.48 | 20.00 | 99.90 |
| 168 | 286.42 | 97.95 | 1.1 | 1 | 4.81 | 23.57 | 99.32 |
| 179 | 275.83 | 100 | 7.5 | 4.2 | 4.51 | 14.77 | 99.50 |
| 186 | 234.22 | 100 | 1.4 | 1.2 | 5.04 | 29.79 | 99.31 |
| 187 | 3.12 | 0 | | | nan | nan | 0.00 |
| 189 | 193.27 | 42 | 3.6 | 3.8 | 5.57 | 34.51 | 96.50 |
| 191 | 271.89 | 100 | 4 | 12 | 5.07 | 28.36 | 95.72 |

PI = polyreactivity for insulin;
PD = polyreactivity for double-stranded DNA;
blank cells indicate no results acquired;
nan = data not available

TABLE 20
Summary of initial screening, binding properties VHH formatted
on Wildtype Fc/modified hinge (SEQ ID NO: 42)

| | HEK-DLL3 | ka (1/Ms) | kd(1/S) | KD nM |
|---|---|---|---|---|
| Clone ID | Internalization FOI | hDLL3 | hDLL3 | hDLL3 |
| 4 | 5.236 | 9.05e+04 | 1.24e−03 | 13.70 |
| 5 | 6.811 | 7.78e+04 | 3.38e−04 | 4.34 |
| 6 | 8.076 | 2.49e+05 | 1.31e−03 | 5.26 |
| 22 | 0.471 | | | |
| 24 | 6.668 | 1.69e+05 | 1.33e−03 | 7.83 |
| 28 | 8.704 | 1.55e+05 | 1.93e−03 | 12.50 |
| 29 | 6.305 | 1.53e+05 | 1.85e−03 | 12.10 |
| 33 | 5.885 | 7.73e+04 | 5.36e−04 | 6.93 |
| 34 | 0.817 | | | |
| 39 | 3.626 | | | |
| 40 | 3.482 | | | |
| 41 | 7.687 | 3.50e+04 | 1.98e−03 | 56.70 |
| 45 | 5.938 | 1.37e+05 | 2.08e−03 | 15.30 |
| 49 | 0.746 | | | |
| 51 | 0.596 | | | |
| 53 | 7.706 | 4.93e+04 | 2.39e−03 | 48.50 |
| 54 | 5.336 | 8.29e+04 | 1.02e−03 | 12.30 |
| 60 | 0.323 | | | |
| 87 | 4.934 | 6.99e+04 | 3.50e−03 | 50.00 |
| 88 | 7.556 | 1.32e+05 | 1.32e−03 | 9.96 |
| 90 | 6.382 | 1.85e+05 | 1.26e−04 | 0.68 |
| 91 | 6.721 | 1.39e+05 | 1.71e−04 | 1.23 |
| 97 | 6.737 | 1.40e+05 | 1.31e−03 | 9.35 |
| 100 | 8.776 | 1.62e+05 | 2.70e−04 | 1.67 |
| 104 | 7.746 | 9.44e+04 | 5.81e−04 | 6.16 |
| 105 | 0.370 | | | |
| 106 | 0.334 | | | |
| 107 | 8.136 | 9.15e+04 | 4.42e−04 | 4.83 |
| 109 | 0.749 | | | |
| 112 | 6.016 | 7.96e+04 | 2.63e−03 | 33.10 |
| 113 | 5.298 | 4.83e+04 | 3.16e−04 | 6.53 |
| 118 | 0.965 | | | |
| 119 | 4.125 | | | |
| 123 | 6.012 | 8.42e+04 | 6.58e−04 | 7.82 |
| 126 | 7.561 | 5.23e+05 | 2.21e−04 | 0.42 |
| 130 | 7.856 | 3.51e+05 | 2.91e−04 | 0.83 |
| 162 | 0.279 | | | |
| 163 | 5.902 | | | |
| 166 | 4.644 | 4.34e+04 | 1.71e−03 | 39.50 |
| 167 | 7.480 | | | |
| 168 | 0.272 | | | |
| 179 | 4.711 | 1.71e+05 | 9.72e−04 | 5.68 |
| 186 | 7.716 | 3.00e+05 | 3.79e−04 | 1.27 |
| 187 | 0.893 | | | |
| 189 | 0.296 | | | |
| 191 | 7.915 | | | |

Blank cells indicate experiments were not performed or data was out of normal range

11 clones of the 46 initially characterized were selected for further characterization. Clones were further characterized by epitope binning, binding affinity to cancer cell lines that naturally express DLL3, in silico immunogenicity analysis, sequence identity to human germline variable region sequences, and developability characteristics. Clones were formatted as VHHT with WT-Fc or Fc with H435Q mutations or Fc with H435Q and ALASS Fc effector mutation. Results from these experiments are shown in Tables 21 and 22.

TABLE 21
in silico characteristics and epitope binning of selected clones

| | | | Treg- | | | |
|---|---|---|---|---|---|---|
| | Immuno- | | adjusted | Identity to | | |
| Clone | genicity | Epivax | Epimatrix | Human | AC- | epitope |
| ID | Risk* | score* | Score* | germline* | SINS* | bin** |
| 5 | None | 5.83 | −18.34 | 84.7 | 8.13 | 1 |
| 6 | Low | 15.56 | −4.32 | 77.2 | 7.27 | 1 |
| 24 | None | 3.05 | −35.61 | 86.1 | 1.58 | 1 |
| 91 | Med | 26.5 | 2.34 | 84.7 | 1.25 | 1 |
| 100 | None | 12.93 | −24.67 | 85.1 | 0.82 | 1 |
| 104 | None | −1.3 | −39.97 | 85.8 | 5.53 | 1 |
| 107 | Low | 5.22 | −3.99 | 84.1 | 7.08 | 3 |
| 119 | High | 65.26 | 65.26 | 77.9 | 2.95 | 1 |
| 123 | Med | 20.67 | −2.89 | 79.6 | 0.67 | 1 |
| 126 | Low | 19.44 | −18.86 | 84.4 | −0.11 | 2 |
| 186 | None | −7.16 | −7.16 | 81.5 | 1.73 | 2 |

*VHH only or
**VHH formatted on Wildtype Fc/modified hinge (SEQ ID NO: 42)

TABLE 22
binding characteristics of selected clones

| | WT-Fc | H435Q-Fc | H435Q-Fc | |
|---|---|---|---|---|
| | SHP-77 | SHP-77 | SHP-77 | H435Q Fc- |
| | Binding | Binding | Internalization | KD (nM) |
| Clone ID | EC50 nM | EC50 nM | FOI | hDLL3 |
| 5 | 2.371 | 4.05 | 1.21 | 0.39 |
| 6 | 0.3853 | 0.22 | 1.16 | 1.17 |
| 24 | 1.99 | 1.2 | 1.19 | 1.08 |
| 91 | 1.97 | 1.52 | 1.16 | <1 |
| 100 | 2.589 | 1.16 | 1.19 | 0.20 |
| 104 | 2.148 | 2.02 | 1.19 | 0.75 |
| 107 | Not done | 0.74 | 1.27 | 0.72 |
| 119 | 1.878 | 1.12 | 1.16 | 3.69 |
| 123 | 92.2 | 67.52 | 1.17 | 1.19 |
| 126 | 1.068 | 0.44 | 1.19 | <1 |
| 186 | 0.4753 | 0.47 | 1.18 | 0.55 |

Several clones were excluded at this stage. For example, clone 123 bound to cancer cells with endogenous DLL3 expression with a binding EC50 that was too high, clones 119 and 91 had a poor immunogenicity score in silico, clones S and 6 had unfavorable (e.g., high ACSINS) score. Based on the data generated in in the further characterization screens S clones representing 3 epitope bins were selected for further analysis. Notably, these epitope bins do not overlap with DLL3 antibody Rovalpituzumab.

VHH-Fcs were tested for internalization by target-expressing cells or target-negative cells. The VHH-Fcs show higher fluorescence signals than the negative control (isotype, expressed as fold over isotype FOI) on target-positive cells, in a dose dependent manner. No internalisation is observed on target negative cells (data not shown). The FOI for 200 nM VHHFc on target positive cells SHP77 is shown in Table 22.

Clones selected for further analysis were: 24 (SEQ ID NO: 101); 100 (SEQ ID NO: 201); 107(SEQ ID NO: 301); 126(SEQ ID NO: 401); and 186 (SEQ ID NO: 501); were selected for further analysis and characterization. These 5 clones were humanized and were tested for binding and developability characteristics after humanization. Humanized variable regions were formatted on an Fc with H435Q, L234A, L235E, G237A, A330S, P331S mutations (Eu numbering).

Humanization was performed to increase identity to human germline sequences and reduce the immunogenic potential of the camelid-derived antibodies. Sequences were modified by grafting the CDRs of a non-human antibody variable region (donor) onto a suitable human framework sequence (acceptor), and selecting a minimal number of key framework residues (back-mutations) from the donor sequence to be incorporated into the acceptor framework sequence to maintain CDR conformation and desired biophysical properties, while minimizing the camelid content of the humanized sequence. Such humanization methods are known in the art, and include those described in Vincke et al., J Biol Chem (2009) (14); Sang et al., Structure (2021) (15) and Moutel et al., Elife (2016) (16). Following in silico design, humanized antibodies were expressed and characterized in vitro to assess retention of functional and biophysical properties

As shown in FIG. 19 humanization of these clones further reduced immunogenicity scores of these clones.

In silico immunogenicity analysis was carried out using the EpiVax algorithm tool called EpiMatrix to predict T cell epitopes presented by human MHC molecules (also known as HLA), which is a prerequisite for immunogenicity. EpiVax tests for binding potential to 9 common human MHC alleles, representative of >95% of human populations worldwide (reference (Jawa et et al Clin Immunol 2013 December; 149(3):534-55.). The platform considers the contribution of regulatory T cell epitopes (Tregitopes) to immunogenic potential also. A protein candidate is ranked against other known immunogenic and non-immunogenic protein sequences. EpiVax suggests proteins with T-regitope-adjusted scores of <−10 at most, and <−20 ideally, should be considered ‘safe’, based on scores from an average of 10 antibodies known to induce anti-therapeutic responses in >500 of patients, and an average of 10 antibodies known to induce anti-therapeutic responses in <5% o of patients.

Immunogenicity scores were determined for all humanized clones and are shown in Table 23.

TABLE 23

| | VHH SEQ | EpiMatrix | tReg Adjusted |
|---|---|---|---|
| | ID NO | Score | Epx Score |
| 24-H | 101 | 3.05 | −35.61 |
| 100-H | 201 | 12.93 | −24.67 |
| 107-H | 301 | 5.22 | −3.99 |
| 126-H | 401 | 19.44 | −18.86 |
| 186-H | 501 | −7.16 | −7.16 |
| 24_ZU1 | 102 | 43.99 | −43.93 |
| 24_ZU2 | 103 | 47.61 | −40.31 |
| 24_ZU3 | 104 | 49.96 | −37.96 |
| 24_ZU4 | 105 | 44.25 | −43.66 |
| 24_ZU5 | 106 | 31.06 | −56.86 |
| 100_ZU1 | 202 | 42.57 | −37.09 |
| 100_ZU2 | 203 | 46.12 | −33.55 |
| 100_ZU3 | 204 | 45.65 | −34.02 |
| 100_ZU4 | 205 | 34.31 | −45.36 |
| 100_ZU5 | 206 | 35.97 | −43.69 |
| 107_ZU1 | 302 | 26.65 | −32.37 |
| 107_ZU2 | 303 | 40.93 | −18.09 |
| 107_ZU3 | 304 | 35.05 | −23.97 |
| 107_ZU4 | 305 | 29.09 | −29.93 |
| 107_ZU5 | 306 | 27.69 | −31.33 |
| 126_ZU1 | 402 | 36.93 | −50.17 |
| 126_ZU2 | 403 | 50.7 | −36.4 |
| 126_ZU3 | 404 | 51.93 | −35.16 |
| 126_ZU4 | 405 | 45.59 | −41.51 |
| 126_ZU5 | 406 | 40.54 | −46.56 |
| 186_ZU1 | 502 | 32.63 | −49.38 |
| 186_ZU2 | 503 | 32.84 | −49.17 |
| 186_ZU3 | 504 | 32.08 | −49.92 |
| 186_ZU4 | 505 | 12.83 | −69.18 |
| 186_ZU5 | 506 | 13.28 | −68.72 |

### Epitope Binning

Antibodies were assessed for binding and competition with each other to determine which epitope bin they belonged to, using Octet. The in-tandem assay was set up using Ni-NTA biosensors, and his-tagged antigens as the ligand, and antibody as the analyte. Ni-NTA biosensors (Cat #: 18-5101) were pre-wet for 10 mins in kinetics buffer (PBS+1% BSA+0.02% Tween 20) at RT. Ni-NTA biosensors were activated with 10 mM NiCl2 for 600s. DLL3.his antigens were captured as ligand for 600s at 10 ug/mL diluted in kinetics buffer. Saturating antibody (Ab1) was allowed to associate for 900s at 250 nM diluted in kinetics buffer. Immediately, competing antibody (Ab2) was allowed to associate for 600s at 250 nM diluted in kinetics buffer. Ni-NTA biosensors were regenerated using 3× cycles of 5s regeneration buffer (10 mM glycine pH 1.5)+5s neutralization (kinetics buffer). Data was analyzed using Octet Data Analysis Software HT 11.1 Epitope Bin operation. Values for Ab pairing were normalized to blank (Ab2 only). Values for Ab pairing were clustered using “Pearson” similarity metric and “Mean” linkage criteria.

### Example 28. Multivalent Constructs

Monospecific and bispecific multivalent constructs exemplified by FIG. 19A were designed targeting FOLR1 (monospecific) or targeting both DLL3 and FOLR1 (bispecific). VHHs were coupled by a linker comprising glycine and serine amino acid residues (e.g., (GGGGS)3, (GGS)4, (GGS)3). FIG. 20 depicts the multivalent constructs described and exemplified herein. The multivalent constructs were made as described herein. Both monospecific and bispecific formats were produced with varying linker lengths. All constructs showed were able to be produced and showed 100% monomeric purification, as shown in Table 25. FOLR1 binder (SEQ ID: 4); DLL3 binder (SEQ ID NO: 8).

TABLE 24

| | | | | Mol. Weight | Yield | % |
|---|---|---|---|---|---|---|
| VHH | Fc | Target | Linker | (Da) | Mg/100 mL | Monomer |
| Anti-FOLR1x4 | Wt | FOLR1 | GGGGSx3 | 103,688 | 23 | 100 |
| Anti-FOLR1x4 | Wt | FOLR1 | GGSx4 | 103,406 | 32 | 100 |
| Anti-FOLR1x4 | Wt | FOLR1 | GGSx3 | 103,004 | 17 | 100 |
| Anti-FOLR1x2 | Wt | FOLR1 | GGGGSx3 | 104,948 | 31 | 100 |
| Anti-DLL3x2 | | DLL3 | | | | |
| Anti-FOLR1x2 | Wt | FOLR1 | GGSx4 | 104,665 | 48 | 100 |
| Anti-DLL3x2 | | DLL3 | | | | |
| Anti-FOLR1x2 | Wt | FOLR1 | GGSx3 | 104,263 | 31 | 100 |
| Anti-DLL3x2 | | DLL3 | | | | |

Multivalent constructs were tested for binding to antigen by a standard ELISA to FOLR1 assay results are shown in Table 25.

TABLE 25

| | hFOLR1 | mFOLR1 |
|---|---|---|
| Sample | EC50 nM | EC50 nM |
| anti-hFOLR1 mIgG MAb (Sinobiologics 11241-MM06) | 0.15 | |
| anti-mFOLR1 RbIgG PAb (Sinobiologics 50573-RP02) | | 0.06 |
| Farletuzumab | 0.12 | nb |
| FolR1 VHH | 0.12 | 0.08 |
| DLL3 VHH | nb | nb |
| Anti-FOLR1x4 GGGGSx3 | 0.12 | 0.08 |
| Anti-FOLR1x4 GGSx4 | 0.14 | 0.11 |
| Anti-FOLR1x4 GGSx3 | 0.11 | 0.09 |
| Anti-FOLR1x2; Anti-DLL3x2; GGGGSx3 | 0.13 | 0.08 |
| Anti-FOLR1x2; Anti-DLL3x2; GGSx4 | 0.11 | 0.07 |
| Anti-FOLR1x2; Anti-DLL3x2; GGSx3 | 0.14 | 0.09 |

### Example 29. Tetramer Binding to FOLR1-Expressing Cancer Cells OVCAR3

Tetramers were tested for binding to FOLR1-positive OVCAR3 cells. Multivalent tetramers having differing linkers and antigen binding domains were tested. All constructs showed similar EC50 to VHH19.

TABLE 26

| Target | EC50 nM |
|---|---|
| FOLR1 Farletuzumab | 0.71 |
| VHH19 | 0.55 |
| Anti-FOLR1x4 (GGGGS)₃ | 0.28 |
| Anti-FOLR1x4 (GGS)₄ | 0.81 |
| Anti-FOLR1x4 (GGS)₃ | 0.49 |
| Anti-FOLR1x2; Anti-DLL3x2; (GGGGS)₃ | 0.70 |
| Anti-FOLR1x2; Anti-DLL3x2; (GGS)₄ | 0.59 |
| Anti-FOLR1x2; Anti-DLL3x2; (GGS)₃ | 0.58 |

### Example 30. Binding to FOLR1 or DLL3 Target Antigen

Tetramers and VHH-Fcs were assessed for affinity to target antigens. AHC Octet biosensors were used to capture tetramer/VHH-Fc and then dipped into antigen. All tetramers bound to human and murine FOLR1 as expected (See Table 27 and 28). The 3 bispecific tetramers were tested for binding to human and murine DLL3 and showed target binding, despite their potentially more restrictive location within the construct design, adjacent to the hinge/Fc.

TABLE 27

| | hFOLR1 | | | | |
|---|---|---|---|---|---|
| | KD (nM) | Ka (1/Ms) | Kd (1/s) | X^{∧}2 | R^{∧}2 |
| Anti-FOLR1x4 (GGGGS)₃ | 2.859 | 3.332 E+05 | 9.526 E−04 | 2.0924 | 0.9976 |
| Anti-FOLR1x4 (GGS)₄ | 2.849 | 2.993 E+05 | 8.526 E−04 | 2.3047 | 0.9977 |
| Anti-FOLR1x4 (GGS)₃ | 2.519 | 3.452 E+05 | 8.695 E−04 | 1.8678 | 0.9973 |
| Anti-FOLR1x2; Anti-DLL3x2; | 2.747 | 4.052 E+05 | 1.113 E−03 | 2.6274 | 0.9961 |
| (GGGGS)₃ | | | | | |
| Anti-FOLR1x2; Anti-DLL3x2; | 2.324 | 4.009 E+05 | 9.318 E−04 | 1.8698 | 0.993 |
| (GGS)₄ | | | | | |
| Anti-FOLR1x2; Anti-DLL3x2; | 2.678 | 3.809 E+05 | 1.02 E−03 | 2.7207 | 0.9947 |
| (GGS)₃ | | | | | |
| FOLR1 VHH | 2.185 | 3.788 E+05 | 8.278 E−04 | 2.377 | 0.9958 |
| DLL3 VHH | No bining | | | | |
| | mFOLR1 | | | | |
| | KD (nM) | Ka (1/Ms) | Kd (1/s) | X^{∧}2 | R^{∧}2 |
| Anti-FOLR1x4 (GGGGS)₃ | 6.595 | 4.709 E+05 | 3.105 E−03 | 2.2162 | 0.9919 |
| Anti-FOLR1x4 (GGS)₄ | 5.999 | 4.351 E+05 | 2.610 E−03 | 2.775 | 0.99 |
| Anti-FOLR1x4 (GGS)₃ | 5.278 | 5.076 E+05 | 2.679 E−03 | 2.3496 | 0.9876 |
| Anti-FOLR1x2; Anti-DLL3x2; | 8.320 | 4.895 E+05 | 4.072 E−03 | 1.2964 | 0.9941 |
| (GGGGS)₃ | | | | | |
| Anti-FOLR1x2; Anti-DLL3x2; | 7.988 | 4.831 E+05 | 3.859 E−03 | 1.7276 | 0.9911 |
| (GGS)₄ | | | | | |
| Anti-FOLR1x2; Anti-DLL3x2; | 7.000 | 5.527 E+05 | 3.986 E−03 | 1.9229 | 0.9889 |
| (GGS)₃ | | | | | |
| FOLR1 VHH | 4.672 | 6.680 E+05 | 3.121 E−03 | 3.3926 | 0.9833 |

| | |
|---|---|
| DLL3 VHH | No binding |

TABLE 28

| | hDLL3 | | | | |
|---|---|---|---|---|---|
| | KD (nM) | Ka (1/Ms) | Kd (1/s) | X?2 | R?2 |
| Anti-FOLR1x2; Anti-DLL3x2; | 1.53 | 2.00E+05 | 3.06E−04 | 4.0815 | 0.9992 |
| (GGGGS)₃ | | | | | |
| Anti-FOLR1x2; Anti-DLL3x2; | 1.24 | 2.15E+05 | 2.67E−04 | 4.9413 | 0.999 |
| (GGS)₄ | | | | | |
| Anti-FOLR1x2; Anti-DLL3x2; | 1.43 | 2.19E+05 | 3.12E−04 | 3.0834 | 0.9994 |
| (GGS)₃ | | | | | |
| DLL3 VHH | 2.29 | 1.66E+05 | 3.79E−04 | 5.497 | 0.9992 |
| FOLR1 VHH | No binding | | | | |
| | mDLL3 | | | | |
| | KD (nM) | Ka (1/Ms) | Kd (1/s) | X?2 | R?2 |
| Anti-FOLR1x4 (GGGGS)₃ | 3.09 | 2.22E+05 | 6.84E−04 | 3.0714 | 0.999 |
| Anti-FOLR1x4 (GGS)₄ | 2.88 | 2.23E+05 | 6.42E−04 | 3.0221 | 0.999 |
| Anti-FOLR1x4 (GGS)₃ | 2.92 | 2.26E+05 | 6.59E−04 | 3.0606 | 0.999 |
| DLL3 VHH | 1.24 | 2.15E+05 | 2.67E−04 | 4.9413 | 0.999 |

| | |
|---|---|
| FOLR1 VHH | No binding |

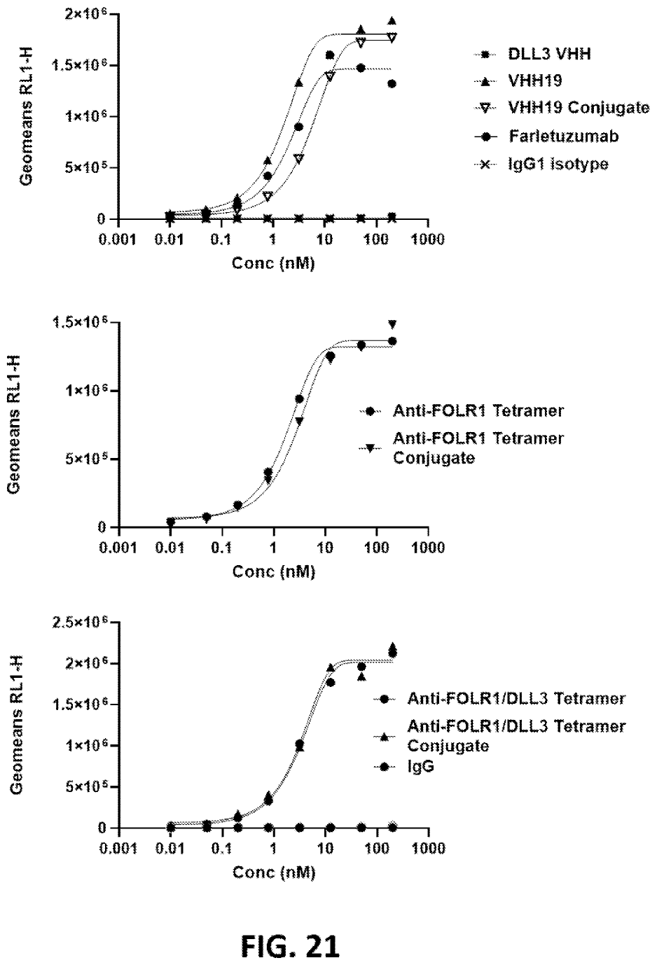

Human/mouse cross-reactive anti-FOLR1 VHHFc, VHH19, was conjugated with p-SCN-Bn-DOTA, as was the monospecific anti-h/mFOLR1 tetramer with (GGS)3 linker and the bispecific anti-h/mFOLR11/h/mDLL3 (GGS)3 tetramer. The human FOLR1 specific VHHFc, VHH17 was conjugated with TFP-Ad-PEG5-DOTA, as described above. Binding to FOLR1 expressing OV-90 cells was confirmed (see FIG. 21).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

SEQUENCES

| SEQ_ID | SEQUENCE |
|---|---|
| 1 | GFTVSNSVMA |
| 2 | IINSIGITNYADSVKG |
| 3 | NFDRIY |
| 4 | QVQLVESGGGLVQPGGSLRLSCAASGFTVSNSVMAWYRQTPGNE |
| | REFVAIINSIGITNYADSVKGRFTISRDNSKNTLYLQMNSLRAE |
| | DTAVYVCNRNFDRIYWGQGTLVTVSS |
| 5 | GSIFSINA |
| 6 | FTGDTNT |
| 7 | AADVQLFSRDYEFY |
| 8 | EVQLVESGGGEVQPGGSLRLSCAASGSIFSINAMGWYRQAPGKQ |
| | RELVAGFTGDTNTIYAESVKGRFTISRDNAKNTVYLQMSSLRAE |
| | DTAVYYCAADVQLFSRDYEFYWGQGTLVTVKP |
| 11 | APELLGGPSVFLFPPKPKDTLMASRTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHYTQKSLSLSPG |
| 12 | APELLGGPSVFLFPPKPKDTLMIARTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHYTQKSLSLSPG |
| 13 | APELLGGPSVFLFPPKPKDTLMISRTPEVTCVVVDVSHEDPEVK |
| | FNWYVDGVEVHNAKTKPREEQYNSTYRVVSVLTVLAQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHYTQKSLSLSPG |
| 14 | APELLGGPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNQYTQKSLSLSPG |
| 15 | APELLGGPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHATQKSLSLSPG |
| 16 | APELLGGPSVFLFPPKPKDTLMISRTPEVTCVVVDVSHEDPEVK |
| | FNWYVDGVEVHNAKTKPREEQYNSTYRVVSVLTVLAQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNQYTQKSLSLSPG |
| 17 | APEAEGAPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPSSIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHYTQKSLSLSPG |
| 18 | APEAEGAPSVFLFPPKPKDTLMISRTPEVTCVVVDVSHEDPEVK |
| | FNWYVDGVEVHNAKTKPREEQYNSTYRVVSVLTVLAQDWLNGKE |
| | YKCKVSNKALPSSIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHYTQKSLSLSPG |
| 19 | APEAEGAPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPSSIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNQYTQKSLSLSPG |
| 20 | APELLGGPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSREEMTKNQ |
| | VSLTCLVKGFYPSDIAVEWESNGQPENNYKTTPPVLDSDGSFFL |
| | YSKLTVDKSRWQQGNVFSCSVMHEALHNHYTQKSLSLSPG |
| 21 | GYIFNSCG |
| 22 | ISGDGDT |
| 23 | AVCYNLETY |
| 24 | QVQLQESGGGSVQAGGSLKLTCAASGYIFNSCGMGWYRQSPGRE |
| | RELVSRISGDGDTWHKESVKGRFTISQDNVKKTLYLQMNSLKPE |
| | DTAVYFCAVCYNLETYWGQGTQVTVSS |
| 40 | EPKSCDKTHTCPPCP |
| 41 | EPKSSDKTHTCPPCP |
| 42 | EPKSSDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAPIEKTISKAKGQPREPQ |
| | VYTLPPSREEMTKNQVSLTCLVKGFYPSDIAVEWESNGQPENNY |
| | KTTPPVLDSDGSFFLYSKLTVDKSRWQQGNVFSCSVMHEALHNH |
| | YTQKSLSLSPG |
| 43 | EPKSSDKTHTCPPCPAPEAEGAPSVFLFPPKPKDTL |
| | MISRTPEVTCVVVDVSHEDPEVKFNWYVDGVEVHNA |
| | KTKPREEQYNSTYRVVSVLTVLHQDWLNGKEYKCKV |
| | SNKALPSSIEKTISKAKGQPREPQVYTLPPSREEMTKN |
| | QVSLTCLVKGFYPSDIAVEWESNGQPENNYKTTPPVL |
| | DSDGSFFLYSKLTVDKSRWQQGNVFSCSVMHEALHN |
| | QYTQKSLSLSPG |
| 101 | QVQYVESGGGLVQPGGSLRLSCAASGFTFSSYRINWYRQP |
| | PGKGRELVGSITDTGSTNYADSVKGRFTISRDNAKNTVYL |
| | QMSSLKPEDTAVYYCRAPTIAAYWGQGTQVTVSS |
| 102 | QVQLVESGGGLVQPGGSLRLSCAASGFTFSSYRINWFRQA |
| | PGQGLEAVASITDTGSTNYADSVKGRFTISRDNSKNTLYL |
| | QMNSLRAEDTAVYYCAAPTIAAYWGQGTLVTVSS |
| 103 | QVQLVESGGGLVQPGGSLRLSCAASGFTFSSYRINWFRQA |
| | PGQGLELVASITDTGSTNYADSVKGRFTISRDNSKNTLYL |
| | QMNSLRAEDTAVYYCAAPTIAAYWGQGTLVTVSS |
| 104 | QVQLVESGGGLVQPGGSLRLSCAASGFTFSSYRINWYRQA |
| | PGQGLELVASITDTGSTNYADSVKGRFTISRDNSKNTLYL |
| | QMNSLRAEDTAVYYCAAPTIAAYWGQGTLVTVSS |
| 105 | QVQLVESGGGLVQPGGSLRLSCAASGFTFSSYRINWYRQA |
| | PGQGRELVASITDTGSTNYADSVKGRFTISRDNSKNTLYL |
| | QMNSLRAEDTAVYYCAAPTIAAYWGQGTLVTVSS |
| 106 | QVQLVESGGGLVQPGGSLRLSCAASGFTFSSYRINWYRQP |
| | PGKGRELVGSITDTGSTNYADSVKGRFTISRDNSKNTLYL |
| | QMNSLRAEDTAVYYCAAPTIAAYWGQGTLVTVSS |
| 107 | GFTFSSYR |
| 108 | SYRIN |
| 109 | GFTESSY |
| 110 | ITDTGST |
| 111 | SITDTGSTNYADSVKG |
| 112 | TDTGS |
| 113 | AAPTIAAY |
| 114 | PTIAAY |
| 115 | PTIAAY |
| 116 | QVQLVESGGGLVQPGGSLRLSCAASGFTFSSYRINWFRQA |
| | PGQGLEAVASITDTGSTNYADSVKGRFTISRDNSKNTLYL |
| | QMNSLRAEDTAVYYCAAPTIAAYWGQGTLVTVSSEPKSSD |
| | KTHTCPPCPAPEAEGAPSVELFPPKPKDTLMISRTPEVTC |
| | VVVDVSHEDPEVKFNWYVDGVEVHNAKTKPREEQYNSTYR |
| | VVSVLTVLHQDWLNGKEYKCKVSNKALPSSIEKTISKAKG |
| | QPREPQVYTLPPSREEMTKNQVSLTCLVKGFYPSDIAVEW |
| | ESNGQPENNYKTTPPVLDSDGSFFLYSKLTVDKSRWQQGN |
| | VFSCSVMHEALHNQYTQKSLSLSPG |
| 117 | QVQLVESGGGLVQPGGSLRLSCAASGFTFSSYRINWERQA |
| | PGQGLELVASITDTGSTNYADSVKGRFTISRDNSKNTLYL |
| | QMNSLRAEDTAVYYCAAPTIAAYWGQGTLVTVSSEPKSSD |
| | KTHTCPPCPAPEAEGAPSVELFPPKPKDTLMISRTPEVTC |
| | VVVDVSHEDPEVKFNWYVDGVEVHNAKTKPREEQYNSTYR |
| | VVSVLTVLHQDWLNGKEYKCKVSNKALPSSIEKTISKAKG |
| | QPREPQVYTLPPSREEMTKNQVSLTCLVKGFYPSDIAVEW |
| | ESNGQPENNYKTTPPVLDSDGSFFLYSKLTVDKSRWQQGN |
| | VFSCSVMHEALHNQYTQKSLSLSPG |
| 118 | QVQLVESGGGLVQPGGSLRLSCAASGFTFSSYRINWYRQA |
| | PGQGLELVASITDTGSTNYADSVKGRFTISRDNSKNTLYL |
| | QMNSLRAEDTAVYYCAAPTIAAYWGQGTLVTVSSEPKSSD |
| | KTHTCPPCPAPEAEGAPSVELFPPKPKDTLMISRTPEVTC |
| | VVVDVSHEDPEVKFNWYVDGVEVHNAKTKPREEQYNSTYR |
| | VVSVLTVLHQDWLNGKEYKCKVSNKALPSSIEKTISKAKG |
| | QPREPQVYTLPPSREEMTKNQVSLTCLVKGFYPSDIAVEW |
| | ESNGQPENNYKTTPPVLDSDGSFFLYSKLTVDKSRWQQGN |
| | VFSCSVMHEALHNQYTQKSLSLSPG |
| 119 | QVQLVESGGGLVQPGGSLRLSCAASGFTFSSYRINWYRQA |
| | PGQGRELVASITDTGSTNYADSVKGRFTISRDNSKNTLYL |
| | QMNSLRAEDTAVYYCAAPTIAAYWGQGTLVTVSSEPKSSD |
| | KTHTCPPCPAPEAEGAPSVELFPPKPKDTLMISRTPEVTC |
| | VVVDVSHEDPEVKENWYVDGVEVHNAKTKPREEQYNSTYR |
| | VVSVLTVLHQDWLNGKEYKCKVSNKALPSSIEKTISKAKG |
| | QPREPQVYTLPPSREEMTKNQVSLTCLVKGFYPSDIAVEW |
| | ESNGQPENNYKTTPPVLDSDGSFFLYSKLTVDKSRWQQGN |
| | VFSCSVMHEALHNQYTQKSLSLSPG |
| 120 | QVQLVESGGGLVQPGGSLRLSCAASGFTFSSYRINWYRQP |
| | PGKGRELVGSITDTGSTNYADSVKGRFTISRDNSKNTLYL |
| | QMNSLRAEDTAVYYCAAPTIAAYWGQGTLVTVSSEPKSSD |
| | KTHTCPPCPAPEAEGAPSVELFPPKPKDTLMISRTPEVTC |
| | VVVDVSHEDPEVKFNWYVDGVEVHNAKTKPREEQYNSTYR |
| | VVSVLTVLHQDWLNGKEYKCKVSNKALPSSIEKTISKAKG |
| | QPREPQVYTLPPSREEMTKNQVSLTCLVKGFYPSDIAVEW |
| | ESNGQPENNYKTTPPVLDSDGSFFLYSKLTVDKSRWQQGN |
| | VFSCSVMHEALHNQYTQKSLSLSPG |
| 201 | QVQLVESGGGSVQPGGSLRLSCAASGIVENSDVMGWYRQV |
| | PGKPRELVATITGGGSTNYATSVKGRFTISRDNAKNTVYL |
| | QMNSLRPEDTAVYYCNGRRGDSMLAFWAQGTQVTVSS |
| 202 | QVQLVESGGGLVQPGGSLRLSCAASGIVENSDVMGWFRQA |
| | PGQGLEAVATITGGGSTNYATSVKGRFTISRDNSKNTLYL |
| | QMNSLRAEDTAVYYCAARRGDSMLAFWGQGTLVTVSS |
| 203 | QVQLVESGGGLVQPGGSLRLSCAASGIVENSDVMGWFRQA |
| | PGQGLELVATITGGGSTNYATSVKGRFTISRDNSKNTLYL |
| | QMNSLRAEDTAVYYCAARRGDSMLAFWGQGTLVTVSS |
| 204 | QVQLVESGGGLVQPGGSLRLSCAASGIVENSDVMGWYRQA |
| | PGQGLELVATITGGGSTNYATSVKGRFTISRDNSKNTLYL |
| | QMNSLRAEDTAVYYCAARRGDSMLAFWGQGTLVTVSS |
| 205 | QVQLVESGGGLVQPGGSLRLSCAASGIVENSDVMGWYRQA |
| | PGQPRELVATITGGGSTNYATSVKGRFTISRDNSKNTLYL |
| | QMNSLRAEDTAVYYCAARRGDSMLAFWGQGTLVTVSS |
| 206 | QVQLVESGGGLVQPGGSLRLSCAASGIVENSDVMGWYRQV |
| | PGKPRELVATITGGGSTNYATSVKGRFTISRDNSKNTLYL |
| | QMNSLRAEDTAVYYCAARRGDSMLAFWGQGTLVTVSS |
| 207 | GIVENSDV |
| 208 | SDVMG |
| 209 | GIVENSD |
| 210 | ITGGGST |
| 211 | TITGGGSTNYATSVKG |
| 212 | TGGGS |
| 213 | AARRGDSMLAF |
| 214 | RRGDSMLAF |
| 215 | RRGDSMLAF |
| 216 | QVQLVESGGGLVQPGGSLRLSCAASGIVENSDVMGWFRQA |
| | PGQGLEAVATITGGGSTNYATSVKGRFTISRDNSKNTLYL |
| | QMNSLRAEDTAVYYCAARRGDSMLAFWGQGTLVTVSSEPK |
| | SSDKTHTCPPCPAPEAEGAPSVELFPPKPKDTLMISRTPE |
| | VTCVVVDVSHEDPEVKENWYVDGVEVHNAKTKPREEQYNS |
| | TYRVVSVLTVLHQDWLNGKEYKCKVSNKALPSSIEKTISK |
| | AKGQPREPQVYTLPPSREEMTKNQVSLTCLVKGFYPSDIA |
| | VEWESNGQPENNYKTTPPVLDSDGSFFLYSKLTVDKSRWQ |
| | QGNVFSCSVMHEALHNQYTQKSLSLSPG |
| 217 | QVQLVESGGGLVQPGGSLRLSCAASGIVENSDVMGWFRQA |
| | PGQGLELVATITGGGSTNYATSVKGRFTISRDNSKNTLYL |
| | QMNSLRAEDTAVYYCAARRGDSMLAFWGQGTLVTVSSEPK |
| | SSDKTHTCPPCPAPEAEGAPSVFLFPPKPKDTLMISRTPE |
| | VTCVVVDVSHEDPEVKFNWYVDGVEVHNAKTKPREEQYNS |
| | TYRVVSVLTVLHQDWLNGKEYKCKVSNKALPSSIEKTISK |
| | AKGQPREPQVYTLPPSREEMTKNQVSLTCLVKGFYPSDIA |
| | VEWESNGQPENNYKTTPPVLDSDGSFFLYSKLTVDKSRWQ |
| | QGNVFSCSVMHEALHNQYTQKSLSLSPG |
| 218 | QVQLVESGGGLVQPGGSLRLSCAASGIVENSDVMGWYRQA |
| | PGQGLELVATITGGGSTNYATSVKGRFTISRDNSKNTLYL |
| | QMNSLRAEDTAVYYCAARRGDSMLAFWGQGTLVTVSSEPK |
| | SSDKTHTCPPCPAPEAEGAPSVELFPPKPKDTLMISRTPE |
| | VTCVVVDVSHEDPEVKFNWYVDGVEVHNAKTKPREEQYNS |
| | TYRVVSVLTVLHQDWLNGKEYKCKVSNKALPSSIEKTISK |
| | AKGQPREPQVYTLPPSREEMTKNQVSLTCLVKGFYPSDIA |
| | VEWESNGQPENNYKTTPPVLDSDGSFFLYSKLTVDKSRWQ |
| | QGNVFSCSVMHEALHNQYTQKSLSLSPG |
| 219 | QVQLVESGGGLVQPGGSLRLSCAASGIVENSDVMGWYRQA |
| | PGQPRELVATITGGGSTNYATSVKGRFTISRDNSKNTLYL |
| | QMNSLRAEDTAVYYCAARRGDSMLAFWGQGTLVTVSSEPK |
| | SSDKTHTCPPCPAPEAEGAPSVFLFPPKPKDTLMISRTPE |
| | VTCVVVDVSHEDPEVKENWYVDGVEVHNAKTKPREEQYNS |
| | TYRVVSVLTVLHQDWLNGKEYKCKVSNKALPSSIEKTISK |
| | AKGQPREPQVYTLPPSREEMTKNQVSLTCLVKGFYPSDIA |
| | VEWESNGQPENNYKTTPPVLDSDGSFFLYSKLTVDKSRWQ |
| | QGNVFSCSVMHEALHNQYTQKSLSLSPG |
| 220 | QVQLVESGGGLVQPGGSLRLSCAASGIVENSDVMGWYRQV |
| | PGKPRELVATITGGGSTNYATSVKGRFTISRDNSKNTLYL |
| | QMNSLRAEDTAVYYCAARRGDSMLAFWGQGTLVTVSSEPK |
| | SSDKTHTCPPCPAPEAEGAPSVELFPPKPKDTLMISRTPE |
| | VTCVVVDVSHEDPEVKENWYVDGVEVHNAKTKPREEQYNS |
| | TYRVVSVLTVLHQDWLNGKEYKCKVSNKALPSSIEKTISK |
| | AKGQPREPQVYTLPPSREEMTKNQVSLTCLVKGFYPSDIA |
| | VEWESNGQPENNYKTTPPVLDSDGSFFLYSKLTVDKSRWQ |
| | QGNVFSCSVMHEALHNQYTQKSLSLSPG |
| 301 | QVQLVESGGGLVQAGDSLRLSCVASDRTESSYAVGWERQA |
| | PGKEREFVAAISWNGGRTLYTDSVTGRFTISRDNAKSTVY |
| | LQMNGLKPEDTAVYYCAARPAAPTRRLEYDYWGQGTQVTV |
| | SS |
| 302 | QVQLVESGGGLVQPGGSLRLSCAASDRTFSSYAVGWFRQA |
| | PGQGLEAVAAISWNGGRTLYTDSVTGRFTISRDNSKNTLY |
| | LQMNSLRAEDTAVYYCAARPAAPTRRLEYDYWGQGTLVTV |
| | SS |
| 303 | QVQLVESGGGLVQPGGSLRLSCAASDRTFSSYAVGWFRQA |
| | PGQGLEFVAAISWNGGRTLYTDSVTGRFTISRDNSKNTLY |
| | LQMNSLRAEDTAVYYCAARPAAPTRRLEYDYWGQGTLVTV |
| | SS |
| 304 | QVQLVESGGGLVQPGGSLRLSCAASDRTFSSYAVGWFRQA |
| | PGQGREFVAAISWNGGRTLYTDSVTGRFTISRDNSKNTLY |
| | LQMNSLRAEDTAVYYCAARPAAPTRRLEYDYWGQGTLVTV |
| | SS |
| 305 | QVQLVESGGGLVQPGGSLRLSCAASDRTESSYAVGWFRQA |
| | PGQEREFVAAISWNGGRTLYTDSVTGRFTISRDNSKNTLY |
| | LQMNSLRAEDTAVYYCAARPAAPTRRLEYDYWGQGTLVTV |
| | SS |
| 306 | QVQLVESGGGLVQPGGSLRLSCAASDRTFSSYAVGWFRQA |
| | PGKEREFVAAISWNGGRTLYTDSVTGRFTISRDNSKNTLY |
| | LQMNSLRAEDTAVYYCAARPAAPTRRLEYDYWGQGTLVTV |
| | SS |
| 307 | DRTFSSYA |
| 308 | SYAVG |
| 309 | DRTFSSY |
| 310 | ISWNGGRT |
| 311 | AISWNGGRTLYTDSVTG |
| 312 | SWNGGR |
| 313 | AARPAAPTRRLEYDY |
| 314 | RPAAPTRRLEYDY |
| 315 | RPAAPTRRLEYDY |
| 316 | QVQLVESGGGLVQPGGSLRLSCAASDRTFSSYAVGWERQA |
| | PGQGLEAVAAISWNGGRTLYTDSVTGRFTISRDNSKNTLY |
| | LQMNSLRAEDTAVYYCAARPAAPTRRLEYDYWGQGTLVTV |
| | SSEPKSSDKTHTCPPCPAPEAEGAPSVELFPPKPKDTLMI |
| | SRTPEVTCVVVDVSHEDPEVKFNWYVDGVEVHNAKTKPRE |
| | EQYNSTYRVVSVLTVLHQDWLNGKEYKCKVSNKALPSSIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNQYTQKSLSLSPG |
| 317 | QVQLVESGGGLVQPGGSLRLSCAASDRTFSSYAVGWERQA |
| | PGQGLEFVAAISWNGGRTLYTDSVTGRFTISRDNSKNTLY |
| | LQMNSLRAEDTAVYYCAARPAAPTRRLEYDYWGQGTLVTV |
| | SSEPKSSDKTHTCPPCPAPEAEGAPSVFLFPPKPKDTLMI |
| | SRTPEVTCVVVDVSHEDPEVKFNWYVDGVEVHNAKTKPRE |
| | EQYNSTYRVVSVLTVLHQDWLNGKEYKCKVSNKALPSSIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNQYTQKSLSLSPG |
| 318 | QVQLVESGGGLVQPGGSLRLSCAASDRTFSSYAVGWERQA |
| | PGQGREFVAAISWNGGRTLYTDSVTGRFTISRDNSKNTLY |
| | LQMNSLRAEDTAVYYCAARPAAPTRRLEYDYWGQGTLVTV |
| | SSEPKSSDKTHTCPPCPAPEAEGAPSVFLFPPKPKDTLMI |
| | SRTPEVTCVVVDVSHEDPEVKFNWYVDGVEVHNAKTKPRE |
| | EQYNSTYRVVSVLTVLHQDWLNGKEYKCKVSNKALPSSIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNQYTQKSLSLSPG |
| 319 | QVQLVESGGGLVQPGGSLRLSCAASDRTFSSYAVGWERQA |
| | PGQEREFVAAISWNGGRTLYTDSVTGRFTISRDNSKNTLY |
| | LQMNSLRAEDTAVYYCAARPAAPTRRLEYDYWGQGTLVTV |
| | SSEPKSSDKTHTCPPCPAPEAEGAPSVFLFPPKPKDTLMI |
| | SRTPEVTCVVVDVSHEDPEVKFNWYVDGVEVHNAKTKPRE |
| | EQYNSTYRVVSVLTVLHQDWLNGKEYKCKVSNKALPSSIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNQYTQKSLSLSPG |
| 320 | QVQLVESGGGLVQPGGSLRLSCAASDRTFSSYAVGWFRQA |
| | PGKEREFVAAISWNGGRTLYTDSVTGRFTISRDNSKNTLY |
| | LQMNSLRAEDTAVYYCAARPAAPTRRLEYDYWGQGTLVTV |
| | SSEPKSSDKTHTCPPCPAPEAEGAPSVFLFPPKPKDTLMI |
| | SRTPEVTCVVVDVSHEDPEVKFNWYVDGVEVHNAKTKPRE |
| | EQYNSTYRVVSVLTVLHQDWLNGKEYKCKVSNKALPSSIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNQYTQKSLSLSPG |
| 401 | QVQLVESGGGMVQPGGSLRLSCAASGITFSMYSMSWYRQP |
| | PGKQRELVAATTTFGSTNYADSVKGRFTISRDNAKNTVYL |
| | QMNSLKPEDTAVYLCNARFTSEEYWGQGTQVTVSS |
| 402 | QVQLVESGGGLVQPGGSLRLSCAASGITFSMYSMSWERQA |
| | PGQGLEAVAATTTFGSTNYADSVKGRFTISRDNSKNTLYL |
| | QMNSLRAEDTAVYYCAARFTSEEYWGQGTLVTVSS |
| 403 | QVQLVESGGGLVQPGGSLRLSCAASGITFSMYSMSWFRQA |
| | PGQGLELVAATTTFGSTNYADSVKGRFTISRDNSKNTLYL |
| | QMNSLRAEDTAVYYCAARFTSEEYWGQGTLVTVSS |
| 404 | QVQLVESGGGLVQPGGSLRLSCAASGITFSMYSMSWYRQA |
| | PGQGLELVAATTTFGSTNYADSVKGRFTISRDNSKNTLYL |
| | QMNSLRAEDTAVYYCAARFTSEEYWGQGTLVTVSS |
| 405 | QVQLVESGGGLVQPGGSLRLSCAASGITFSMYSMSWYRQA |
| | PGQQRELVAATTTFGSTNYADSVKGRFTISRDNSKNTLYL |
| | QMNSLRAEDTAVYYCAARFTSEEYWGQGTLVTVSS |
| 406 | QVQLVESGGGLVQPGGSLRLSCAASGITFSMYSMSWYRQP |
| | PGKQRELVAATTTFGSTNYADSVKGRFTISRDNSKNTLYL |
| | QMNSLRAEDTAVYYCAARFTSEEYWGQGTLVTVSS |
| 407 | GITESMYS |
| 408 | MYSMS |
| 409 | GITFSMY |
| 410 | TTTFGST |
| 411 | ATTTFGSTNYADSVKG |
| 412 | TTFGS |
| 413 | AARFTSEEY |
| 414 | RFTSEEY |
| 415 | RFTSEEY |
| 416 | QVQLVESGGGLVQPGGSLRLSCAASGITFSMYSMSWERQA |
| | PGQGLEAVAATTTFGSTNYADSVKGRFTISRDNSKNTLYL |
| | QMNSLRAEDTAVYYCAARFTSEEYWGQGTLVTVSSEPKSS |
| | DKTHTCPPCPAPEAEGAPSVFLFPPKPKDTLMISRTPEVT |
| | CVVVDVSHEDPEVKFNWYVDGVEVHNAKTKPREEQYNSTY |
| | RVVSVLTVLHQDWLNGKEYKCKVSNKALPSSIEKTISKAK |
| | GQPREPQVYTLPPSREEMTKNQVSLTCLVKGFYPSDIAVE |
| | WESNGQPENNYKTTPPVLDSDGSFFLYSKLTVDKSRWQQG |
| | NVFSCSVMHEALHNQYTQKSLSLSPG |
| 417 | QVQLVESGGGLVQPGGSLRLSCAASGITFSMYSMSWFRQA |
| | PGQGLELVAATTTFGSTNYADSVKGRFTISRDNSKNTLYL |
| | QMNSLRAEDTAVYYCAARFTSEEYWGQGTLVTVSSEPKSS |
| | DKTHTCPPCPAPEAEGAPSVFLFPPKPKDTLMISRTPEVT |
| | CVVVDVSHEDPEVKFNWYVDGVEVHNAKTKPREEQYNSTY |
| | RVVSVLTVLHQDWLNGKEYKCKVSNKALPSSIEKTISKAK |
| | GQPREPQVYTLPPSREEMTKNQVSLTCLVKGFYPSDIAVE |
| | WESNGQPENNYKTTPPVLDSDGSFFLYSKLTVDKSRWQQG |
| | NVFSCSVMHEALHNQYTQKSLSLSPG |
| 418 | QVQLVESGGGLVQPGGSLRLSCAASGITFSMYSMSWYRQA |
| | PGQGLELVAATTTFGSTNYADSVKGRFTISRDNSKNTLYL |
| | QMNSLRAEDTAVYYCAARFTSEEYWGQGTLVTVSSEPKSS |
| | DKTHTCPPCPAPEAEGAPSVFLFPPKPKDTLMISRTPEVT |
| | CVVVDVSHEDPEVKFNWYVDGVEVHNAKTKPREEQYNSTY |
| | RVVSVLTVLHQDWLNGKEYKCKVSNKALPSSIEKTISKAK |
| | GQPREPQVYTLPPSREEMTKNQVSLTCLVKGFYPSDIAVE |
| | WESNGQPENNYKTTPPVLDSDGSFFLYSKLTVDKSRWQQG |
| | NVFSCSVMHEALHNQYTQKSLSLSPG |
| 419 | QVQLVESGGGLVQPGGSLRLSCAASGITFSMYSMSWYRQA |
| | PGQQRELVAATTTFGSTNYADSVKGRFTISRDNSKNTLYL |
| | QMNSLRAEDTAVYYCAARFTSEEYWGQGTLVTVSSEPKSS |
| | DKTHTCPPCPAPEAEGAPSVFLFPPKPKDTLMISRTPEVT |
| | CVVVDVSHEDPEVKFNWYVDGVEVHNAKTKPREEQYNSTY |
| | RVVSVLTVLHQDWLNGKEYKCKVSNKALPSSIEKTISKAK |
| | GQPREPQVYTLPPSREEMTKNQVSLTCLVKGFYPSDIAVE |
| | WESNGQPENNYKTTPPVLDSDGSFFLYSKLTVDKSRWQQG |
| | NVFSCSVMHEALHNQYTQKSLSLSPG |
| 420 | QVQLVESGGGLVQPGGSLRLSCAASGITFSMYSMSWYRQP |
| | PGKQRELVAATTTFGSTNYADSVKGRFTISRDNSKNTLYL |
| | QMNSLRAEDTAVYYCAARFTSEEYWGQGTLVTVSSEPKSS |
| | DKTHTCPPCPAPEAEGAPSVELFPPKPKDTLMISRTPEVT |
| | CVVVDVSHEDPEVKFNWYVDGVEVHNAKTKPREEQYNSTY |
| | RVVSVLTVLHQDWLNGKEYKCKVSNKALPSSIEKTISKAK |
| | GQPREPQVYTLPPSREEMTKNQVSLTCLVKGFYPSDIAVE |
| | WESNGQPENNYKTTPPVLDSDGSFFLYSKLTVDKSRWQQG |
| | NVFSCSVMHEALHNQYTQKSLSLSPG |
| 501 | QVQVVESGGGLVQPGGSLTLSCAASGFDFSTYTVNWYRQA |
| | PGKEREKVARISSTGTTTNYANSAKGREFLSRDNAKNRVY |
| | LQMNNLKPEDTAVYFCNFERFDSNYWGQGTQVTVSS |
| 502 | QVQLVESGGGLVQPGGSLRLSCAASGFDFSTYTVNWFRQA |
| | PGQGLEAVARISSTGTTTNYANSAKGRFTISRDNSKNTLY |
| | LQMNSLRAEDTAVYYCAAERFDSNYWGQGTLVTVSS |
| 503 | QVQLVESGGGLVQPGGSLRLSCAASGFDFSTYTVNWFRQA |
| | PGQGLEKVARISSTGTTTNYANSAKGRFTISRDNSKNTLY |
| | LQMNSLRAEDTAVYYCAAERFDSNYWGQGTLVTVSS |
| 504 | QVQLVESGGGLVQPGGSLRLSCAASGFDFSTYTVNWYRQA |
| | PGQGLEKVARISSTGTTTNYANSAKGRFTISRDNSKNTLY |
| | LQMNSLRAEDTAVYYCAAERFDSNYWGQGTLVTVSS |
| 505 | QVQLVESGGGLVQPGGSLRLSCAASGFDFSTYTVNWYRQA |
| | PGQEREKVARISSTGTTTNYANSAKGRFTISRDNSKNTLY |
| | LQMNSLRAEDTAVYYCAAERFDSNYWGQGTLVTVSS |
| 506 | QVQLVESGGGLVQPGGSLRLSCAASGFDFSTYTVNWYRQA |
| | PGKEREKVARISSTGTTTNYANSAKGRFTISRDNSKNTLY |
| | LQMNSLRAEDTAVYYCAAERFDSNYWGQGTLVTVSS |
| 507 | GFDFSTYT |
| 508 | TYTVN |
| 509 | GFDESTY |
| 510 | ISSTGTTT |
| 511 | RISSTGTTTNYANSAKG |
| 512 | SSTGTT |
| 513 | AAERFDSNY |
| 514 | AAERFDSNY |
| 515 | ERFDSNY |
| 516 | QVQLVESGGGLVQPGGSLRLSCAASGFDFSTYTVNWFRQA |
| | PGQGLEAVARISSTGTTTNYANSAKGRFTISRDNSKNTLY |
| | LQMNSLRAEDTAVYYCAAERFDSNYWGQGTLVTVSSEPKS |
| | SDKTHTCPPCPAPEAEGAPSVFLFPPKPKDTLMISRTPEV |
| | TCVVVDVSHEDPEVKFNWYVDGVEVHNAKTKPREEQYNST |
| | YRVVSVLTVLHQDWLNGKEYKCKVSNKALPSSIEKTISKA |
| | KGQPREPQVYTLPPSREEMTKNQVSLTCLVKGFYPSDIAV |
| | EWESNGQPENNYKTTPPVLDSDGSFFLYSKLTVDKSRWQQ |
| | GNVFSCSVMHEALHNQYTQKSLSLSPG |
| 517 | QVQLVESGGGLVQPGGSLRLSCAASGFDFSTYTVNWFRQA |
| | PGQGLEKVARISSTGTTTNYANSAKGRFTISRDNSKNTLY |
| | LQMNSLRAEDTAVYYCAAERFDSNYWGQGTLVTVSSEPKS |
| | SDKTHTCPPCPAPEAEGAPSVFLFPPKPKDTLMISRTPEV |
| | TCVVVDVSHEDPEVKFNWYVDGVEVHNAKTKPREEQYNST |
| | YRVVSVLTVLHQDWLNGKEYKCKVSNKALPSSIEKTISKA |
| | KGQPREPQVYTLPPSREEMTKNQVSLTCLVKGFYPSDIAV |
| | EWESNGQPENNYKTTPPVLDSDGSFFLYSKLTVDKSRWQQ |
| | GNVFSCSVMHEALHNQYTQKSLSLSPG |
| 518 | QVQLVESGGGLVQPGGSLRLSCAASGFDFSTYTVNWYRQA |
| | PGQGLEKVARISSTGTTTNYANSAKGRFTISRDNSKNTLY |
| | LQMNSLRAEDTAVYYCAAERFDSNYWGQGTLVTVSSEPKS |
| | SDKTHTCPPCPAPEAEGAPSVFLFPPKPKDTLMISRTPEV |
| | TCVVVDVSHEDPEVKFNWYVDGVEVHNAKTKPREEQYNST |
| | YRVVSVLTVLHQDWLNGKEYKCKVSNKALPSSIEKTISKA |
| | KGQPREPQVYTLPPSREEMTKNQVSLTCLVKGFYPSDIAV |
| | EWESNGQPENNYKTTPPVLDSDGSFFLYSKLTVDKSRWQQ |
| | GNVFSCSVMHEALHNQYTQKSLSLSPG |
| 519 | QVQLVESGGGLVQPGGSLRLSCAASGFDFSTYTVNWYRQA |
| | PGQEREKVARISSTGTTTNYANSAKGRFTISRDNSKNTLY |
| | LQMNSLRAEDTAVYYCAAERFDSNYWGQGTLVTVSSEPKS |
| | SDKTHTCPPCPAPEAEGAPSVFLFPPKPKDTLMISRTPEV |
| | TCVVVDVSHEDPEVKFNWYVDGVEVHNAKTKPREEQYNST |
| | YRVVSVLTVLHQDWLNGKEYKCKVSNKALPSSIEKTISKA |
| | KGQPREPQVYTLPPSREEMTKNQVSLTCLVKGFYPSDIAV |
| | EWESNGQPENNYKTTPPVLDSDGSFFLYSKLTVDKSRWQQ |
| | GNVFSCSVMHEALHNQYTQKSLSLSPG |
| 520 | QVQLVESGGGLVQPGGSLRLSCAASGFDFSTYTVNWYRQA |
| | PGKEREKVARISSTGTTTNYANSAKGRFTISRDNSKNTLY |
| | LQMNSLRAEDTAVYYCAAERFDSNYWGQGTLVTVSSEPKS |
| | SDKTHTCPPCPAPEAEGAPSVELFPPKPKDTLMISRTPEV |
| | TCVVVDVSHEDPEVKFNWYVDGVEVHNAKTKPREEQYNST |
| | YRVVSVLTVLHQDWLNGKEYKCKVSNKALPSSIEKTISKA |
| | KGQPREPQVYTLPPSREEMTKNQVSLTCLVKGFYPSDIAV |
| | EWESNGQPENNYKTTPPVLDSDGSFFLYSKLTVDKSRWQQ |
| | GNVFSCSVMHEALHNQYTQKSLSLSPG |

1.-74. (canceled)

75. An immunoconjugate comprising a multivalent antibody and a chelating agent, wherein the multivalent antibody comprises a polypeptide having the structure of Formula I: 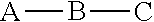
wherein:
A comprises a first VHH;
B comprises a second VHH;
C comprises an Fc domain; and
wherein the Fc domain comprises an alteration to one or more amino acid residues that alters binding of the immunoconjugate to the neonatal Fc receptor (FcRn).

76. The immunoconjugate of claim 75, wherein the Fc domain comprises an alteration to one or more amino acid residues that reduces an effector function of the Fc domain.

77. The immunoconjugate of claim 76, wherein the alteration to one or more amino acid residues that reduces the effector function of the immunoglobulin heavy chain constant region comprises L234A, L235E, G237A, A330S, and P331S per EU numbering.

78. The immunoconjugate of claim 75, wherein the alteration to one or more amino acid residues that alters binding of the immunoconjugate to the neonatal Fc receptor (FcRn) is to an amino acid residue selected from the list consisting of: I253A, H310A, H435Q, and combinations thereof per EU numbering.

79. The immunoconjugate of claim 75, wherein the alteration to one or more amino acid residues that alters binding of the immunoconjugate to the neonatal Fc receptor (FcRn) comprises I253A per EU numbering.

80. The immunoconjugate of claim 75, wherein the alteration to one or more amino acid residues that alters binding of the immunoconjugate to the neonatal Fc receptor (FcRn) comprises H310A per EU numbering.

81. The immunoconjugate of claim 75, wherein the alteration to one or more amino acid residues that alters binding of the immunoconjugate to the neonatal Fc receptor (FcRn) comprises H435Q per EU numbering.

82. The immunoconjugate of claim 75, wherein the chelating agent is a linker-chelator selected from the list consisting of: TFP-Ad-PEG5-DOTAGA, p-SCN-Bn-DOTA, p-SCN-Ph-Et-Py4Pa, and TFP-Ad-PEG5-Ac-Py4Pa.

83. The immunoconjugate of claim 75, further comprising a radioisotope, wherein the radioisotope is an alpha emitter or a beta emitter.

84. The immunoconjugate of claim 83, wherein the radioisotope is 225-Ac.

85. A method of treating a cancer or a tumor in an individual comprising administering to the individual the immunoconjugate of claim 75, thereby treating the cancer or the tumor.

86. A method of imaging a tumor in an individual comprising administering to the individual the immunoconjugate of claim 75.

87. A nucleic acid encoding the polypeptide of claim 75.

88. An expression vector comprising the nucleic acid of claim 87.

89. A cell comprising the nucleic acid of claim 87 or the expression vector of claim 88.
